(19) United States
(12) Patent Application Publication
(10) Pub. No.: US 2022/0162312 A1
(43) Pub. Date: May 26, 2022

(54) NOVEL BISPECIFIC CD3/CD20 POLYPEPTIDE COMPLEXES

(71) Applicant: WUXI BIOLOGICS IRELAND LIMITED, Dublin (IE)

(72) Inventors: Yunying CHEN, Shanghai (CN); Qin MEI, Shanghai (CN); Jianqing XU, Shanghai (CN); Zhuozhi WANG, Shanghai (CN); Jing LI, Shanghai (CN)

(21) Appl. No.: 17/425,870

(22) PCT Filed: Jan. 22, 2020
(86) PCT No.: PCT/CN2020/073687

§ 371 Date: Jul. 26, 2021

(30) Foreign Application Priority Data
Jan. 28, 2019 (CN) PCT/CN2019/073418

(51) Int. Cl. C07K 16/28; A61P 35/00; C07K 14/725; C07K 14/705

(52) U.S. Cl. CPC C07K 16/2809; A61P 35/00; A61K 2039/505; C07K 14/70596; C07K 16/2887; C07K 14/7051

(57) ABSTRACT

The present disclosure provides a bispecific anti-CD3×CD20 polypeptide complex that contains a first antigen-binding moiety of the polypeptide complex and a second antigen-binding moiety, methods of producing the bispecific anti-CD3×CD20 polypeptide complex, methods of treating disease or disorder using the bispecific anti-CD3×CD20 polypeptide complex, polynucleotides encoding the bispecific anti-CD3×CD20 polypeptide complex, vectors and host cells containing said polynucleotides, and compositions and pharmaceutical compositions comprising the bispecific anti-CD3×CD20 polypeptide complex.

### PRIORITY CLAIM

The present application claims the priority of PCT/CN2019/073418 filed on Jan. 28,2019, which was incorporated to the present disclosure as an entirety by reference.

### SEQUENCE LISTING

The present application is filed with a Sequence Listing in electronic form. The entire contents of the Sequence Listing are hereby incorporated by reference.

### FIELD OF INVENTION

The present disclosure generally relates to bispecific anti-CD3×CD20 polypeptide complexes.

### BACKGROUND

Bispecific antibodies are growing to be the new category of therapeutic antibodies. They can bind two different targets or two different epitopes on a target, creating additive or synergistic effect superior to the effect of individual antibodies. A lot of antibody engineering efforts have been put into designing new bispecific formats, such as DVD-Ig, CrossMab, BiTE etc. (Spiess et al., Molecular Immunology, 67(2), pp. 95-106 (2015)). However, these formats may potentially have various limitations in stability, solubility, short half-life, and immunogenicity.

Among these bispecific antibody formats, an IgG-like bispecific antibody is a common format: one arm binding to target A and another arm binding to target B. Structurally it is made from half of antibody A and half of antibody B, with the similar size and shape as a natural IgG. In order to facilitate downstream development, it is desired that such bispecific molecules can be easily produced like normal IgG from a single host cell with high expression level and correctly assembled form. Unfortunately, the pairing of cognate light-heavy chains as well as the assembly of two different half antibodies cannot be automatically controlled. All kinds of mispairings in a random manner could result in significant product heterogeneity.

By introducing mutations in the Fc region, such as “knobs-into-holes” (Ridgway et al., Protein Engineering, 9(7), pp. 617-21(1996); Merchant et al., Nature Biotechnology, 16(7), pp. 677-681(1998)), electrostatics (Gunasekaran et al., Journal of Biological Chemistry, 285(25), pp. 19637-19646 (2010)) or negative state designs (Von Kreudenstein et al., mAbs, 5(5), pp. 646-654 (2013); Leaver-Fay et al., Structure, 24(4), pp. 641-651 (2016)), the preferred heterodimeric assembly of two different heavy chains has been accomplished. However, the selective pairing of light-heavy chains of each individual antibody remains challenging. The interface between light-heavy chains includes the variable domain (VH-VL) and the constant domain (CH1-CL). Several strategies have been applied into designing orthogonal interfaces to facilitate cognate pairing. Roche swapped the domains of CH1 and CL and created the CrossMab platform (Schaefer et al., Proceedings of the National Academy of Sciences of the United States of America, 108(27), pp. 11187-11192 (2011)), MedImmune introduced alternatively disulphide bond (Mazor et al., mAbs, 7(2), pp. 377-389 (2015)), Amgen made further electrostatics in the CH1-CL region (Liu et al., Journal of Biological Chemistry, 290(12), pp. 7535-7562 (2015)), and Lilly (Lewis et al., Nature Biotechnology, 32(2), pp. 191-198 (2014)) and Genentech (Dillon et al., mAbs, 9(2), pp. 213-230 (2017)) introduced mutations in both variable and constant domains.

CD20 is an activated-glycosylated phosphoprotein expressed on the surface of B-lymphocytes. Antibody therapy with Rituximab, a chimeric anti-CD20 monoclonal antibody (also referred to as “mAb” hereinafter) approved by FDA in 1997, represents one of the most important progress in the treatment of lymphoproliferative disorders in the last 30 years. Particularly, in the combination with various chemotherapy/radiotherapy regimes, Rituximab has significantly improved all aspects of the survival statistics of B cell lymphoma and chronic lymphoid lymphoma (CLL) patients (Chu T W, Zhang R, Yang J, et al. A Two-Step Pretargeted Nanotherapy for CD20 Crosslinking May Achieve Superior Anti-Lymphoma Efficacy to Rituximab. Theranostics. 2015 Apr. 26; 5(8): 834-46).

During the past three decades, people made considerable progress in understanding of the protein structure and molecular function of CD20, therefore the new generation of anti-CD20 therapeutic antibodies have been generated and approved for clinical usage. Ofatumumab is a fully human anti-CD20 therapeutic antibody, which targets a different CD20 epitope of greater proximity to cell surface than Rituximab, resulting in a slower off-rate and more stable binding than Rituximab (Laurenti L, Innocenti I, Autore F, et al. New developments in the management of chronic lymphocytic leukemia: role of ofatumumab. Onco Targets Ther. 2016 Jan. 20; 9: 421-9). Nevertheless, the new generation of anti-CD20 monoclonal antibodies were not proven to be more significantly superior than Rituximab in efficacy and safety. For anti-CD20 mAb treatments, disease relapse or recurrence will still occur to all patients with follicular lymphoma and CLL, and about half of patients with aggressive B cell lymphoma, for example, diffuse large B cell lymphoma (Lim S H, Beers S A, French R R, et al. Anti-CD20 monoclonal antibodies: historical and future perspectives. Haematologica. 2010 January; 95(1):135-43). Thus, an unmet medical need is remained to develop new strategy of B cell-targeting therapeutics with distinct mechanism of action (MOA), such as bispecific antibody and chimeric antigen receptors (CARs)-T cell treatments.

The CD3 T-cell co-receptor is a protein complex composed of four distinct chains, a CD3gamma chain, a CD3delta chain, and two CD3epsilon chains. The four chains associate with a molecule known as T-cell receptor (TCR) and the zeta-chain to generate activation signal in T lymphocytes. The TCR, zetachain, and CD3 molecules compose the TCR complex, in which TCR as a subunit recognizes and binds to antigen, and CD3 as a subunit transfers and conveys the antigen stimulation to signaling pathway, and ultimately regulates T-cell activity. The CD3 protein is present in virtually all T cells. The CD3-TCR complex modulates T cell functions in both innate and adoptive immune response, as well as cellular and humoral immune functions. These include eliminating pathogenic organisms and controlling tumor growth by broad range of cytotoxic effects. Mouse monoclonal antibodies specific for human CD3, such as OKT3 (Kung et al., Science, 206: 347-9 (1979)), were the first generation CD3 antibodies developed for treatment. Although OKT3 has strong immunosuppressive potency, its clinical use was hampered by serious side effects linked to its immunogenic and mitogenic potentials (Chatenoud, Nature Reviews, 3:123-132 (2003)). OKT3 induced an anti-globulin response, promoting its own rapid clearance and neutralization (Chatenoud et al., Eur. J. Immunol., 137:830-8 (1982)). In addition, OKT3 induced T-cell proliferation and cytokine production in vitro, and led to a large-scale release of cytokine in vivo (Hirsch et al., J. Immunol, 142: 737-43 (1989)). Such serious side effects limited the more widespread use of OKT3 in transplantation as well as the extension of its use to other clinical fields such as autoimmunity.

A bispecific antibody targeting CD3 and CD20 can bind to T cells and B cells simultaneously. Once the bispecific antibody binds to a CD3-positive T cell and a CD20-positive B cell, a cytolytic synapse is formed. Cytotoxicity is then induced by the release of perforin and granzymes from granules in the cytotoxic T cell, the latter inducing apoptosis and lysis of the malignant B cell.

There is great need to design bispecific molecules to both CD3 and CD20. Such bispecific anti-CD3×CD20 polypeptide complexes are useful for treating CD20-related conditions including cancer.

### BRIEF SUMMARY OF INVENTION

In one aspect, the present disclosure provides a bispecific polypeptide complex, comprising a first antigen-binding moiety associated with a second antigen-binding moiety, wherein:

the first antigen-binding moiety comprising:
a first polypeptide comprising, from N-terminus to C-terminus, a first heavy chain variable domain (VH) of a first antibody operably linked to a first T cell receptor (TCR) constant region (C1), and
a second polypeptide comprising, from N-terminus to C-terminus, a first light chain variable domain (VL) of the first antibody operably linked to a second TCR constant region (C2),
wherein:
C1 and C2 are capable of forming a dimer comprising at least one non-native interchain bond between C1 and C2, and the non-native interchain bond is capable of stabilizing the dimer
and

the second antigen-binding moiety comprising:
a second heavy chain variable domain (VH2) of a second antibody operably linked to an antibody heavy chain CH1 domain, and
a second light chain variable domain (VL2) of the second antibody operably linked to an antibody light chain constant (CL) domain,

wherein:
one of the first and the second antigen-binding moiety is an anti-CD3 binding moiety, and the other one is an anti-CD20 binding moiety,
the anti-CD3 binding moiety is derived from an anti-CD3 antibody comprising:
   a) a heavy chain CDR1 comprising an amino acid sequence selected from the group consisting of SEQ ID NO: 1, 13, 25, 37 and 49,
   b) a heavy chain CDR2 comprising an amino acid sequence selected from the group consisting of SEQ ID NO: 2, 14, 26, 38 and 50,
   c) a heavy chain CDR3 comprising an amino acid sequence selected from the group consisting of SEQ ID NO: 3, 15, 27, 39 and 51,
   d) a kappa light chain CDR1 comprising an amino acid sequence selected from the group consisting of SEQ ID NO: 4, 16, 28, 40 and 52, e) a kappa light chain CDR2 comprising an amino acid sequence selected from the group consisting of SEQ ID NO: 5, 17, 29, 41 and 53, and
   f) a kappa light chain CDR3 comprising an amino acid sequence selected from the group consisting of SEQ ID NO: 6, 18, 30, 42 and 54,
the anti-CD20 binding moiety is derived from an anti-CD20 antibody comprising:
   a) a heavy chain CDR1 comprising an amino acid sequence selected from the group consisting of SEQ ID NO: 7, 19, 31, 43 and 55,
   b) a heavy chain CDR2 comprising an amino acid sequence selected from the group consisting of SEQ ID NO: 8, 20, 32, 44 and 56,
   c) a heavy chain CDR3 comprising an amino acid sequence selected from the group consisting of SEQ ID NO: 9, 21, 33, 45 and 57,
   d) a kappa light chain CDR1 comprising an amino acid sequence selected from the group consisting of SEQ ID NO: 10, 22, 34, 46 and 58,
   e) a kappa light chain CDR2 comprising an amino acid sequence selected from the group consisting of SEQ ID NO: 11, 23, 35, 47 and 59, and
   f) a kappa light chain CDR3 comprising an amino acid sequence selected from the group consisting of SEQ ID NO: 12, 24, 36, 48 and 60.

In certain embodiments, the anti-CD3 binding moiety of the bispecific polypeptide complex is derived from an anti-CD3 antibody comprising a heavy chain variable domain sequence comprising an amino acid sequence selected from the group consisting of SEQ ID NO: 61, 63, 65, 67 and 69 and a light chain variable domain sequence comprising an amino acid sequence selected from the group consisting of SEQ ID NO: 62, 64, 66, 68 and 70.

In certain embodiments, the anti-CD20 binding moiety of the bispecific polypeptide complex is derived from an anti-CD20 antibody comprising a heavy chain variable domain sequence comprising SEQ ID NO: 71, 73,75, 77 and 79 and a light chain variable domain sequence comprising SEQ ID NO: 72, 74, 76, 78 and 80.

In certain embodiments, the bispecific polypeptide complex comprises a combination of four polypeptide sequences: SEQ ID NO: 81, SEQ ID NO: 82, SEQ ID NO: 83, and SEQ ID NO: 84.

In certain embodiments, the bispecific polypeptide complex comprises a combination of four polypeptide sequences: SEQ ID NO: 85, SEQ ID NO: 86, SEQ ID NO: 87, and SEQ ID NO: 88.

In certain embodiments, the bispecific polypeptide complex comprises a combination of four polypeptide sequences: SEQ ID NO: 89, SEQ ID NO: 90, SEQ ID NO: 91, and SEQ ID NO: 92.

In certain embodiments, the bispecific polypeptide complex comprises a combination of four polypeptide sequences: SEQ ID NO: 93, SEQ ID NO: 94, SEQ ID NO: 95, and SEQ ID NO: 96.

In certain embodiments, the bispecific polypeptide complex comprises a combination of four polypeptide sequences: SEQ ID NO: 97, SEQ ID NO: 98, SEQ ID NO: 99, and SEQ ID NO: 100.

In certain embodiments, one or more amino acids from the natural glycosylation site at positions 182, 193, 203, 206 and 207 in the polypeptide sequence of SEQ ID NO: 92 are modified. Preferably, the modification is made to the amino acid at position 193 of SEQ ID NO: 92. In certain embodiments, such modification includes one or more mutations of S182X, S193X, S203X, S206X or S207X, wherein X represents any amino acid other than Ser and Thr. In certain preferred embodiments, said modification is S193X, wherein X is selected from Ala, Gly, Pro or Val. In certain embodiments, the above mutation(s) remove an O-glycosylation site, and the type of 0-glycosylation is O-saccharide in a Corel configuration and has a structural formula of NeuAc-Gal-GalNAc or NeuAc-Gal-(NeuAc) GalNAc.

In one aspect, the present disclosure provides a bispecific polypeptide complex having one or more of the following properties:

(a) specifically binding to human CD3 and CD20 protein simultaneously with an appropriate affinity;

(b) specifically binding to human CD3 and/or cyno CD3 protein;

(c) specifically binding to human CD20 and/or cyno CD20 protein;

(d) in some in vitro cellular funtionological experiments, capable of inducing more potent T cell activation, more effectively and specifically killing CD20 positive tumor cells, and releasing less number of cytokines, as compared to other bispecific antibodies targeting CD3 and CD20;

(e) having good thermal stability and being stable in cyno or human serum;

(f) providing superior in vivo anti-tumor effect compared to other bispecific antibodies targeting CD3 and CD20;

(g) exhibiting an effect of effectively depeleting B cells and a sufficient serum half-life (T_{1/2}) without adverse reactions such as cytokine storm and the like in cynomolgus monkeys. In one aspect, the present disclosure provides herein a conjugate comprising the bispecific polypeptide complex provided herein conjugated to a moiety.

In one aspect, the present disclosure provides herein an isolated polynucleotide encoding the bispecific polypeptide complex provided herein.

In one aspect, the present disclosure provides herein an isolated vector comprising the polynucleotide provided herein.

In one aspect, the present disclosure provides herein a host cell comprising the isolated polynucleotide provided herein or the isolated vector provided herein.

In one aspect, the present disclosure provides herein a method of expressing the bispecific polypeptide complex provided herein, comprising culturing the host cell provided herein under the condition at which the bispecific polypeptide complex is expressed.

In one aspect, the present disclosure provides a composition comprising the bispecific polypeptide complex provided herein.

In one aspect, the present disclosure provides herein a pharmaceutical composition comprising the bispecific polypeptide complex provided herein and a pharmaceutically acceptable carrier.

In one aspect, the present disclosure provides herein a method of treating a CD20-related disease or condition in a subject in need thereof, comprising administrating to the subject a therapeutically effective amount of the bispecific polypeptide complex provided herein. In certain embodiments, the disease or condition can be alleviated, eliminated, treated, or prevented when the first antigen and the second antigen are both modulated.

In certain embodiments, the first antigen-binding moiety is linked to a first dimerization domain, and the second antigen-binding moiety is linked to a second dimerization domain, wherein the first and the second dimerization domains are associated. In certain embodiments, the association is via a connecter, a disulphide bond, a hydrogen bond, electrostatic interaction, a salt bridge, or hydrophobic-hydrophilic interaction, or the combination thereof.

In certain embodiments, the first and/or the second dimerization domain comprises at least a portion of an antibody hinge region, optionally derived from IgG1, IgG2 or IgG4.

In certain embodiments, the second dimerization domain is operably linked to the heavy chain variable domain of the second antigen-binding moiety.

In certain embodiments, the first and the second dimerization domains are different and associate in a way that discourages homodimerization and/or favors heterodimerization.

In certain embodiments, the first and the second dimerization domains are capable of associating into heterodimers via knobs-into-holes, hydrophobic interaction, electrostatic interaction, hydrophilic interaction, or increased flexibility.

In another aspect, the present disclosure provides a kit comprising the polypeptide complex provided herein for detection, diagnosis, prognosis, or treatment of a disease or condition.

In another aspect, the present disclosure provides use of the bispecific polypeptide complex provided herein in the manufacture for treating a CD20-related disease or condition in a subject.

In an embodiment, the CD20-related disease or condition is cancer, preferably, the cancer is selected from, but not limited to, lymphoma, lung cancer, liver cancer, cervical cancer, colon cancer, breast cancer, ovarian cancer, pancreatic cancer, melanoma, glioblastoma, prostate cancer, esophageal cancer or gastric cancer.

In an embodiment, the CD20-related disease or condition is B cell lymphoma, optionally Hodgkin lymphoma or non-Hodgkin lymphoma, wherein the non-Hodgkin lymphoma comprises: Diffuse large B-cell lymphoma (DLBCL), Follicular lymphoma, Marginal zone B-cell lymphoma (MZL), Mucosa-Associated Lymphatic Tissue lymphoma (MALT), Small lymphocytic lymphoma (chronic lymphocytic leukemia, CLL), or Mantle cell lymphoma (MCL), Acute Lymphoblastic Leukemia (ALL), or Waldenstrom's Macroglobulinemia (WM).

The foregoing and other features and advantages of the invention will become more apparent from the following detailed description of several embodiments which proceeds with reference to the accompanying figures.

### BRIEF DESCRIPTION OF FIGURES

FIG. 1 presents schematic representations of studied antibody formats, in which “E17R-1”, “F16-1” and “F17R-1” schematically represent the format of the following bispecific antibodies: W3278-T2U3.E17R-1.uIgG4.SP, W3278-T3U2.F16-1.uIgG4. SP and W3278-T3U2.F17R-1.uIgG4. SP, respectively, and “F18R-1” schematically represents the format of W3278-U2T3.F18R-1.uIgG4. SP and W3278-U3 T2.F18R-1.uIgG4. SP. In the context of the present disclosure, “U” in the nominature of the bispecific antibodies refers to anti-CD20 antibody or anti-CD20 binding moity, and “T” in the nominature of the bispecific antibodies refers to anti-CD3 antibody or anti-CD3 binding moity. The constant region (CL and CH1) of “T” was replaced by the constant domains of TCR to design unique light-heavy chain interface that is orthogonal to regular antibody. The TCR-modified “T” and native “U” in conjunction with “knobs-into-holes” mutations in Fc domain were used to design bispecific antibody formats “E17R-1”, “F16-1,” “F17R-1” and “F18R-1.”

FIG. 2 shows Target Binding by FACS: (A) WBP3278 BsAbs binding test on Jurkat cells, and (B) WBP3278 BsAbs binding test on Raji cells.

FIG. 3 shows simultaneous dual target binding by FACS.

FIG. 4 shows T Cell Killing.

FIGS. 5A and 5B show T Cell Activation indicated by CD25 expression and by CD69 expression, respectively.

FIG. 6 shows IL-2 (A) and TNF-α (B) release by CD4⁺ T cells.

FIG. 7 shows the result of serum stability.

FIG. 8 shows the dose-dependent anti-tumor activity of BsAb of the present disclosure in in vivo therapeutic treatment model.

FIG. 9 shows the depletion of circulating B cells in peripheral blood in naïve male cynomolgus monkeys which were administrated by WBP3278 lead Ab (i.e., W3278-U2T3.F18R-1.uIgG4).

FIGS. 10A, 10B and 10C shown the changes in the levels of circulating T cells in peripheral blood after WBP3278 lead Ab treatment.

FIG. 11 shows the changes in the levels of circulating cytokines after WBP3278 lead Ab treatment.

FIG. 12 shows the changes in serum concentration of the WBP3278 lead Ab over time.

### DETAILED DESCRIPTION OF INVENTION

The following description of the disclosure is merely intended to illustrate various embodiments of the disclosure.

### Definitions

The articles “a,” “an,” and “the” are used herein to refer to one or to more than one (i.e., to at least one) of the grammatical object of the article. By way of example, “a polypeptide complex” means one polypeptide complex or more than one polypeptide complex.

As used herein, the term “about” or “approximately” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this disclosure, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this disclosure to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The terms “polypeptide,” “peptide,” and “protein” are used interchangeably herein to refer to a polymer of amino acid residues, or an assembly of multiple polymers of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an alpha-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. An alpha-carbon refers to the first carbon atom that attaches to a functional group, such as a carbonyl. A beta-carbon refers to the second carbon atom linked to the alpha-carbon, and the system continues naming the carbons in alphabetical order with Greek letters. Amino acid mimetics refers to chemical compounds that have a structure that is different from the general chemical structure of an amino acid, but that functions in a manner similar to a naturally occurring amino acid. The term “protein” typically refers to large polypeptides. The term “peptide” typically refers to short polypeptides. Polypeptide sequences are usually described as the left-hand end of a polypeptide sequence is the amino-terminus (N-terminus); the right-hand end of a polypeptide sequence is the carboxyl-terminus (C-terminus). “Polypeptide complex” as used herein refers to a complex comprising one or more polypeptides that are associated to perform certain functions. In certain embodiments, the polypeptides are immune-related.

The term “antibody” as used herein encompasses any immunoglobulin, monoclonal antibody, polyclonal antibody, multispecific antibody, or bispecific (bivalent) antibody that binds to a specific antigen. A native intact antibody comprises two heavy chains and two light chains. Each heavy chain consists of a variable region (“HCVR”) and a first, second, and third constant region (CH1, CH2 and CH3), while each light chain consists of a variable region (“LCVR”) and a constant region (CL). Mammalian heavy chains are classified as α, δ, ε, γ, and μ, and mammalian light chains are classified as λ or κ. The antibody has a “Y” shape, with the stem of the Y consisting of the second and third constant regions of two heavy chains bound together via disulphide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light (L) chain CDRs including LCDR1, LCDR2, and LCDR3, heavy (H) chain CDRs including HCDR1, HCDR2, HCDR3). CDR boundaries for antibodies may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. Each HCVR and LCVR comprises four FRs, and the CDRs and FRs are arranged from amino terminus to carboxy terminus in the order: FR1, CDR1, FR2, CDR2, FR3, CDR3, FR4. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “variable domain” with respect to an antibody as used herein refers to an antibody variable region or a fragment thereof comprising one or more CDRs. Although a variable domain may comprise an intact variable region (such as HCVR or LCVR), it is also possible to comprise less than an intact variable region yet still retain the capability of binding to an antigen or forming an antigen-binding site.

The term “antigen-binding moiety” as used herein refers to an antibody fragment formed from a portion of an antibody comprising one or more CDRs, or any other antibody fragment that binds to an antigen but does not comprise an intact native antibody structure. Examples of antigen-binding moiety include, without limitation, a variable domain, a variable region, a diabody, a Fab, a Fab′, a F(ab′)₂, an Fv fragment, a disulphide stabilized Fv fragment (dsFv), a (dsFv)₂, a bispecific dsFv (dsFv-dsFv′), a disulphide stabilized diabody (ds diabody), a multispecific antibody, a camelized single domain antibody, a nanobody, a domain antibody, and a bivalent domain antibody. An antigen-binding moiety is capable of binding to the same antigen to which the parent antibody binds. In certain embodiments, an antigen-binding moiety may comprise one or more CDRs from a particular human antibody grafted to a framework region from one or more different human antibodies. For more and detailed formats of antigen-binding moiety are described in Spiess et al, 2015 (Supra), and Brinkman et al., mAbs, 9(2), pp. 182-212 (2017), which are incorporated herein by their entirety.

“Fab” with regard to an antibody refers to that portion of the antibody consisting of a single light chain (both variable and constant regions) associating to the variable region and first constant region of a single heavy chain by a disulphide bond. In certain embodiments, the constant regions of both the light chain and heavy chain are replaced with TCR constant regions.

“Fab′” refers to a Fab fragment that includes a portion of the hinge region.

“F(ab′)₂” refers to a dimer of Fab′.

A “fragment difficult (Fd)” with regard to an antibody refers to the amino-terminal half of the heavy chain fragment that can be combined with the light chain to form Fab.

“Fc” with regard to an antibody refers to that portion of the antibody consisting of the second (CH2) and third (CH3) constant regions of a first heavy chain bound to the second and third constant regions of a second heavy chain via disulphide bonding. The Fc portion of the antibody is responsible for various effector functions such as ADCC, and CDC, but does not function in antigen binding.

“Hinge region” in terms of an antibody includes the portion of a heavy chain molecule that joins the CH1 domain to the CH2 domain. This hinge region comprises approximately 25 amino acid residues and is flexible, thus allowing the two N-terminus antigen binding regions to move independently.

“CH2 domain” as used herein refers to includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat, E., et al., U.S. Department of Health and Human Services, (1983)).

The “CH3 domain” extends from the CH2 domain to the C-terminus of the IgG molecule and comprises approximately 108 amino acids. Certain immunoglobulin classes, e.g., IgM, further include a CH4 region.

“Fv” with regard to an antibody refers to the smallest fragment of the antibody to bear the complete antigen binding site. An Fv fragment consists of the variable domain of a single light chain bound to the variable domain of a single heavy chain. A number of Fv designs have been provided, including dsFvs, in which the association between the two domains is enhanced by an introduced disulphide bond; and scFvs can be formed using a peptide linker to bind the two domains together as a single polypeptide. Fvs constructs containing a variable domain of a heavy or light immunoglobulin chain associated to the variable and constant domain of the corresponding immunoglobulin heavy or light chain have also been produced. Fvs have also been multimerised to form diabodies and triabodies (Maynard et al., Annu Rev Biomed Eng 2 339-376 (2000)).

“ScFab” refers to a fusion polypeptide with a Fd linked to a light chain via a polypeptide linker, resulting in the formation of a single chain Fab fragment (scFab).

“TriFabs” refers to a trivalent, bispecific fusion protein composed of three units with Fab-functionalities. TriFabs harbor two regular Fabs fused to an asymmetric Fab-like moiety.

“Fab-Fab” refers to a fusion protein formed by fusing the Fd chain of a first Fab arm to the N-terminus of the Fd chain of a second Fab arm.

“Fab-Fv” refers to a fusion protein formed by fusing a HCVR to the C-terminus of a Fd chain and a LCVR to the C-terminus of a light chain. A “Fab-dsFv” molecule can be formed by introducing an interdomain disulphide bond between the HCVR domain and the LCVR domain.

“MAb-Fv” or “IgG-Fv” refers to a fusion protein formed by fusion of HCVR domain to the C-terminus of one Fc chain and the LCVR domain either expressed separately or fused to the C-terminus of the other resulted in a bispecific, trivalent IgG-Fv (mAb-Fv) fusion protein, with the Fv stabilized by an interdomain disulphide bond.

“ScFab-Fc-scFv₂” and “ScFab-Fc-scFv” refer to a fusion protein formed by fusion of a single-chain Fab with Fc and disulphide-stabilized Fv domains.

“Appended IgG” refers to a fusion protein with a Fab arm fused to an IgG to form the format of bispecific (Fab)₂-Fc. It can form a “IgG-Fab” or a “Fab-IgG”, with a Fab fused to the C-terminus or N-terminus of an IgG molecule with or without a connector. In certain embodiments, the appended IgG can be further modified to a format of IgG-Fab₄ (see, Brinkman et al., 2017, Supra).

“DVD-Ig” refers to a dual-variable-domain antibody that is formed by fusion of an additional HCVR domain and LCVR domain of a second specificity to an IgG heavy chain and light chain. “CODV-Ig” refers to a related format where the two HCVR and two LCVR domains are linked in a way that allows crossover pairing of the variable HCVR-LCVR domains, which are arranged either (from N- to C-terminus) in the order HCVRA-HCVRB and LCVRB-LCVRA, or in the order HCVRB-HCVRA and LCVRA-LCVRB.

A “CrossMab” refers to a technology of pairing of unmodified light chain with the corresponding unmodified heavy chain and pairing of the modified light chain with the corresponding modified heavy chain, thus resulting an antibody with reduced mispairing in the light chain.

A “BiTE” is a bispecific T-cell engager molecule, comprising a first scFv with a first antigen specificity in the LCVR-HCVR orientation linked to a second scFv with a second specificity in the HCVR-LCVR orientation.

A “WuXiBody” is a bispecific antibody comprising soluble chimeric protein with variable domains of an antibody and the constant domains of TCR, wherein the subunits (such as alpha and beta domains) of TCR constant domains are linked by engineered disulfide bond.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

An “antigen” or “Ag” as used herein refers to a compound, composition, peptide, polypeptide, protein or substance that can stimulate the production of antibodies or a T cell response in cell culture or in an animal, including compositions (such as one that includes a cancer-specific protein) that are added to a cell culture (such as a hybridoma), or injected or absorbed into an animal. An antigen reacts with the products of specific humoral or cellular immunity (such as an antibody), including those induced by heterologous antigens.

An “epitope” or “antigenic determinant” refers to the region of an antigen to which a binding agent (such as an antibody) binds. Epitopes can be formed both from contiguous amino acids (also called linear or sequential epitope) or noncontiguous amino acids juxtaposed by tertiary folding of a protein (also called configurational or conformational epitope). Epitopes formed from contiguous amino acids are typically arranged linearly along the primary amino acid residues on the protein and the small segments of the contiguous amino acids can be digested from an antigen binding with major histocompatibility complex (MHC) molecules or retained on exposure to denaturing solvents whereas epitopes formed by tertiary folding are typically lost on treatment with denaturing solvents. An epitope typically includes at least 3, and more usually, at least 5, about 7, or about 8-10 amino acids in a unique spatial conformation.

The term “specific binding” or “specifically binds” as used herein refers to a non-random binding reaction between two molecules, such as for example between an antibody and an antigen. In certain embodiments, the polypeptide complex and the bispecific polypeptide complex provided herein specifically bind an antigen with a binding affinity (K_{D}) of ≤10⁻⁶ M (e.g., ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤2×10⁻⁹ M, ≤10⁻⁹ M, or ≤10⁻¹⁰ M). K_{D} as used herein refers to the ratio of the dissociation rate to the association rate (k_{off}/kₒₙ), may be determined using surface plasmon resonance methods for example using instrument such as Biacore.

The term “operably link” or “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “fusion” or “fused” when used with respect to amino acid sequences (e.g. peptide, polypeptide or protein) refers to combination of two or more amino acid sequences, for example by chemical bonding or recombinant means, into a single amino acid sequence which does not exist naturally. A fusion amino acid sequence may be produced by genetic recombination of two encoding polynucleotide sequences, and can be expressed by a method of introducing a construct containing the recombinant polynucleotides into a host cell.

The term “spacer” as used herein refers to an artificial amino acid sequence having 1, 2, 3, 4 or 5 amino acid residues, or a length of between 5 and 15, 20, 30, 50 or more amino acid residues, joined by peptide bonds and are used to link one or more polypeptides. A spacer may or may not have a secondary structure. Spacer sequences are known in the art, see, for example, Holliger et al., Proc. Natl. Acad. Sci. USA 90:6444-6448 (1993); Poljak et al., Structure 2:1121-1123 (1994). Any suitable spacers known in the art can be used.

The term “antigenic specificity” refers to a particular antigen or an epitope thereof that is selectively recognized by an antigen-binding molecule.

The term “substitution” with regard to amino acid residue as used herein refers to naturally occurring or induced replacement of one or more amino acids with another in a peptide, polypeptide or protein. Substitution in a polypeptide may result in diminishment, enhancement, or elimination of the polypeptide's function.

Substitution can also be “conservative substitution” with reference to amino acid sequence refers to replacing an amino acid residue with a different amino acid residue having a side chain with similar physiochemical properties or substitution of those amino acids that are not critical to the activity of the polypeptide. For example, conservative substitutions can be made among amino acid residues with nonpolar side chains (e.g., Met, Ala, Val, Leu, and Ile, Pro, Phe, Trp), among residues with uncharged polar side chains (e.g., Cys, Ser, Thr, Asn, Gly and Gln), among residues with acidic side chains (e.g. Asp, Glu), among amino acids with basic side chains (e.g., His, Lys, and Arg), among amino acids with beta-branched side chains (e.g., Thr, Val and Ile), among amino acids with sulfur-containing side chains (e.g., Cys and Met), or among residues with aromatic side chains (e.g., Trp, Tyr, His and Phe). In certain embodiments, substitutions, deletions or additions can also be considered as “conservative substitution”. The number of amino acids that are inserted or deleted can be in the range of about 1 to 5. Conservative substitution usually does not cause significant change in the protein conformational structure, and therefore could retain the biological activity of a protein.

The term “mutation” or “mutated” with regard to amino acid residue as used herein refers to substitution, insertion, or addition of an amino acid residue.

“T cell” as used herein refers to a type of lymphocyte that plays a critical role in the cell-mediated immunity, including helper T cells (e.g. CD4⁺ T cells, T helper 1 type T cells, T helper 2 type T cells, T helper 3 type T cells, T helper 17 type T cells), cytotoxic T cells (e.g. CD8⁺ T cells), memory T cells (e.g. central memory T cells (TCM cells), effector memory T cells (TEM cells and TEMRA cells) and resident memory T cells (TRM) that are either CD8+ or CD4+), natural killer T (NKT) cells and inhibitory T cells.

A native “T cell receptor” or a native “TCR” is a heterodimeric T cell surface protein which is associated with invariant CD3 chains to form a complex capable of mediating signal transduction. TCR belongs to the immunoglobulin superfamily, and is similar to a half antibody with a single heavy chain and a single light chain. Native TCR has an extracellular portion, a transmembrane portion and an intracellular portion. The extracellular domain of a TCR has a membrane-proximal constant region and a membrane-distal variable region.

The term “subject” or “individual” or “animal” or “patient” as used herein refers to human or non-human animal, including a mammal or a primate, in need of diagnosis, prognosis, amelioration, prevention and/or treatment of a disease or disorder. Mammalian subjects include humans, domestic animals, farm animals, and zoo, sports, or pet animals such as dogs, cats, guinea pigs, rabbits, rats, mice, horses, swine, cows, bears, and so on.

Bispecific Polypeptide Complex

In one aspect, the present disclosure provides herein a bispecific polypeptide complex. The term “bispecific” as used herein means that there are two antigen-binding moieties, each of which is capable of specifically binding to a different antigen or a different epitope on the same antigen. The bispecific polypeptide complex provided herein comprises a first antigen-binding moiety associated with a second antigen-binding moiety, and one of them specifically binds to CD3, and the other specifically binds to CD20. In other words, the first antigen-binding moiety may specifically bind to CD3 and the second antigen-binding moiety may specifically bind to CD20. Alternatively, the first antigen-binding moiety may specifically bind to CD20 and the second antigen-binding moiety may specifically bind to CD3. In the present disclosure, the terms “bispecific anti-CD3×CD20 polypeptide complex”, “a polypeptide complex targeting CD3 and CD20” or “anti-CD3 and CD20 polypeptide complex” can be used interchangeably.

In certain embodiments, the present disclosure provides a bispecific polypeptide complex, comprising a first antigen-binding moiety associated with a second antigen-binding moiety, wherein:
the first antigen-binding moiety comprising:
a first polypeptide comprising, from N-terminus to C-terminus, a first heavy chain variable domain (VH) of a first antibody operably linked to a first T cell receptor (TCR) constant region (C1), and
a second polypeptide comprising, from N-terminus to C-terminus, a first light chain variable domain (VL) of the first antibody operably linked to a second TCR constant region (C2),
wherein:
C1 and C2 are capable of forming a dimer comprising at least one non-native interchain bond between C1 and C2, and the non-native interchain bond is capable of stabilizing the dimer
and
the second antigen-binding moiety comprising:
a second heavy chain variable domain (VH2) of a second antibody operably linked to an antibody heavy chain CH1 domain, and
a second light chain variable domain (VL2) of the second antibody operably linked to an antibody light chain constant (CL) domain,
wherein:
one of the first and the second antigen-binding moiety is an anti-CD3 binding moiety, and the other one is an anti-CD20 binding moiety, the anti-CD3 binding moiety is derived from an anti-CD3 antibody comprising:
a) a heavy chain CDR1 comprising an amino acid sequence selected from the group consisting of SEQ ID NO: 1, 13, 25, 37 and 49,
b) a heavy chain CDR2 comprising an amino acid sequence selected from the group consisting of SEQ ID NO: 2, 14, 26, 38 and 50,
c) a heavy chain CDR3 comprising an amino acid sequence selected from the group consisting of SEQ ID NO: 3, 15, 27, 39 and 51,
d) a kappa light chain CDR1 comprising an amino acid sequence selected from the group consisting of SEQ ID NO: 4, 16, 28, 40 and 52,
e) a kappa light chain CDR2 comprising an amino acid sequence selected from the group consisting of SEQ ID NO: 5, 17, 29, 41 and 53, and
f) a kappa light chain CDR3 comprising an amino acid sequence selected from the group consisting of SEQ ID NO: 6, 18, 30, 42 and 54,
the anti-CD20 binding moiety is derived from an anti-CD20 antibody comprising:
a) a heavy chain CDR1 comprising an amino acid sequence selected from the group consisting of SEQ ID NO: 7, 19, 31, 43 and 55,
b) a heavy chain CDR2 comprising an amino acid sequence selected from the group consisting of SEQ ID NO: 8, 20, 32, 44 and 56,
c) a heavy chain CDR3 comprising an amino acid sequence selected from the group consisting of SEQ ID NO: 9, 21, 33, 45 and 57,
d) a kappa light chain CDR1 comprising an amino acid sequence selected from the group consisting of SEQ ID NO: 10, 22, 34, 46 and 58,
e) a kappa light chain CDR2 comprising an amino acid sequence selected from the group consisting of SEQ ID NO: 11, 23, 35, 47 and 59, and
f) a kappa light chain CDR3 comprising an amino acid sequence selected from the group consisting of SEQ ID NO: 12, 24, 36, 48 and 60.

In certain embodiments, the bispecific polypeptide complex provided herein comprises a first antigen-binding moiety containing a sequence derived from a TCR constant region but the second antigen-binding moiety does not contain a sequence derived from a TCR constant region.

The bispecific polypeptide complex provided herein is significantly less prone to have mispaired heavy chain and light chain variable domains. Without wishing to be bound by any theory, it is believed that the stabilized TCR constant regions in the first antigen-binding moiety can specifically associate with each other and therefore contribute to the highly specific pairing of the intended VH1 and VL1, while discouraging unwanted mispairings of VH1 or VL1 with other variable regions that do not provide for the intended antigen-binding sites.

In certain embodiments, the second antigen-binding moiety further comprises an antibody constant CH1 domain operably linked to VH2, and an antibody light chain constant domain operably linked to VL2. Thus, the second antigen-binding moiety comprises a Fab.

Where the first, second, third, and fourth variable domains (e.g. VH1, VH2, VL1 and VL2) are expressed in one cell, it is highly desired that VH1 specifically pairs with VL1, and VH2 specifically pairs with VL2, such that the resulting bispecific protein product would have the correct antigen-binding specificities. However, in existing technologies such as hybrid-hybridoma (or quadroma), random pairing of VH1, VH2, VL1 and VL2 occurs and consequently results in generation of up to ten different species, of which only one is the functional bispecific antigen-binding molecule. This not only reduces production yields but also complicates the purification of the target product.

The bispecific polypeptide complexes provided herein are exceptional in that the variable domains are less prone to mispair than otherwise would have been if both the first and the second antigen-binding moieties are counterparts of natural Fab. In an illustrative example, the first antigen-binding domain comprises VH1-C1 paired with VL1-C2, and the second antigen-binding domain comprises VH2-CH1 paired with VL2-CL. It has been surprisingly found that C1 and C2 preferentially associates with each other, and are less prone to associate with CL or CH1, thereby formation of unwanted pairs such as C1-CH, C1-CL, C2-CH, and C2-CL are discouraged and significantly reduced. As a result of specific association of C1-C2, VH1 specifically pairs with VL1, thereby rendering the first antigen binding site, and CH1 specifically pairs with CL, thereby allowing specific pairing of VH2-VL2 which provides for the second antigen binding site. Accordingly, the first antigen binding moiety and the second antigen binding moiety are less prone to mismatch, and mispairings between for example VH1-VL2, VH2-VL1, VH1-VH2, VL1-VL2 are significantly reduced than otherwise could have been if both the first and the second antigen-binding moieties are counterparts of natural Fabs, e.g. in the form of VH1-CH1, VL1-CL, VH2-CH1, and VL2-CL.

In certain embodiments, the bispecific polypeptide complex provided herein, when expressed from a cell, has significantly less mispairing products (e.g., at least 1, 2, 3, 4, 5 or more mispairing products less) and/or significantly higher production yield (e.g., at least 10%, 20%, 30%, 40%, 50%, 60% or more higher yield), than a reference molecule expressed under comparable conditions, wherein the reference molecule is otherwise identical to the bispecific polypeptide complex except having a native CH1 in the place of C1 and a native CL in the place of C2.

Antigen-Binding Moiety Comprising Engineered CAlpha and CBeta

The first antigen-binding moiety provided herein comprises a first antibody heavy chain variable domain operably linked to a first T cell receptor (TCR) constant region, and a first antibody light chain variable domain operably linked to a second TCR constant region, wherein the first TCR constant region and the second TCR constant region are associated via at least one non-native interchain disulphide bond. The first antigen-binding moiety comprises at least two polypeptide chains, each of which comprises a variable domain derived from an antibody and a constant region derived from a TCR. Thus, the first antigen-binding moiety comprises a heavy chain variable domain and a light chain variable domain, which are operably linked to a pair of TCR constant regions, respectively. In certain embodiments, the pair of TCR constant regions in the first antigen-binding moiety are alpha/beta TCR constant regions. The TCR constant regions in the polypeptide complexes provided herein are capable of associating with each other to form a dimer through at least one non-native disulphide bond.

It is surprisingly found that the first antigen-binding moiety provided herein with at least one non-native disulphide bond can be recombinantly expressed and assembled into the desired conformation, which stabilizes the TCR constant region dimer while providing for good antigen-binding activity of the antibody variable regions. Moreover, the first antigen-binding moiety is found to well tolerate routine antibody engineering, for example, modification of glycosylation sites, and removal of some natural sequences. Furthermore, the polypeptide complexes provided herein can be incorporated into a bispecific format which can be readily expressed and assembled with minimal or substantially no mispairing of the antigen-binding sequences due to the presence of the TCR constant regions in the first antigen-binding moiety. Additional advantages of the first antigen-binding moiety and constructs provided herein will become more evident in the following disclosure below.

In summary, the first antigen-binding moiety provided herein comprises a first polypeptide comprising, from N-terminus to C-terminus, a first heavy chain variable domain (VH) of a first antibody operably linked to a first T cell receptor (TCR) constant region (C1), and a second polypeptide comprising, from N-terminus to C-terminus, a first light chain variable domain (VL) of the first antibody operably linked to a second TCR constant region (C2), wherein C1 and C2 are capable of forming a dimer, and the non-native interchain disulphide bond between C1 and C2 is capable of stabilizing the dimer.

TCR Constant Region

The first antigen-binding moiety provided herein comprises an alpha or beta constant region derived from a TCR.

Human TCR alpha chain constant region is known as TRAC, with the NCBI accession number of P01848.

Human TCR beta chain constant region has two different variants, known as TRBC1 and TRBC2 (IMGT nomenclature) (see also Toyonaga B, et al., PNAs, Vol. 82, pp. 8624-8628, Immunology (1985)).

In the present disclosure, the first and the second TCR constant regions of the first antigen-binding moiety provided herein are capable of forming a dimer comprising, between the TCR constant regions, at least one non-native interchain disulphide bond that is capable of stabilizing the dimer.

The term “dimer” as used herein refers to an associated structure formed by two molecules, such as polypeptides or proteins, via covalent or non-covalent interactions. A homodimer or homodimerization is formed by two identical molecules, and a heterodimer or heterodimerization is formed by two different molecules. The dimer formed by the first and the second TCR constant regions is a heterodimer.

A “mutated” amino acid residue refers to one which is substituted, inserted or added and is different from its native counterpart residue in a corresponding native TCR constant region. For example, if an amino acid residue at a particular position in the wild-type TCR constant region is referred to as the “native” residue, then its mutated counterpart is any residue that is different from the native residue but resides at the same position on the TCR constant region. A mutated residue can be a different residue which substitutes the native residue at the same position, or which is inserted before the native residue and therefore takes up its original position.

In the polypeptide complexes provided herein, the first and/or the second TCR constant regions have been engineered to comprise one or more mutated amino acid residues that are responsible for forming the non-native interchain disulphide bond. To introduce such a mutated residue to the TCR constant region, an encoding sequence of a TCR region can be manipulated to for example, substitute a codon encoding a native residue for the codon encoding the mutated residue, or to insert a codon encoding the mutated residue before the codon of the native residue.

In the polypeptide complexes provided herein, the first and/or the second TCR constant regions have been engineered to comprise one or more mutated cysteine residues such that, after replacement to cysteine residues, a non-native interchain disulphide bond could be formed between the two TCR constant regions.

The non-native interchain disulphide bond is capable of stabilizing the first antigen-binding moiety. Such effects in stabilization can be embodied in various ways. For example, the presence of the mutated amino acid residue or the non-native interchain disulphide bond can enable the polypeptide complex to stably express, and/or to express in a high level, and/or to associate into a stable complex having the desired biological activity (e.g. antigen binding activity), and/or to express and assemble into a high level of desired stable complex having the desired biological activity. The capability of the interchain disulphide bond to stabilize the first and the second TCR constant regions can be assessed using proper methods known in the art, such as the molecular weight displayed on SDS-PAGE, or thermostability measured by differential scanning calorimetry (DSC) or differential scanning fluorimetry (DSF). In an illustrative example, formation of a stable first antigen-binding moiety provided herein can be confirmed by SDS-PAGE, if a product shows a molecular weight comparable to the combined molecular weight of the first and the second polypeptides. In certain embodiments, the first antigen-binding moiety provided herein is stable in that its thermal stability is no less than 50%, 60%, 70%, 80%, or 90% of that of a natural Fab. In certain embodiments, the first antigen-binding moiety provided herein is stable in that its thermal stability is comparable to that of a natural Fab.

Without wishing to be bound by any theory, it is believed that the non-native interchain disulphide bond formed between the first and the second TCR constant regions in the first antigen-binding moiety are capable of stabilizing the heterodimer of TCR constant regions, thereby enhancing the level of correct folding, the structural stability and/or the expression level of the heterodimer and of the first antigen-binding moiety. Unlike native TCR anchored on the membrane of T cell surface, heterodimers of native TCR extracellular domains are found to be much less stable, despite of its similarity to antibody Fab in 3D structure. As a matter of fact, the instability of native TCR in soluble condition used to be a significant obstacle that prevents elucidation of its crystal structure (see Wang, Protein Cell, 5(9), pp. 649-652 (2014)). By introducing a pair of Cysteine (Cys) mutations in TCR constant regions and thereby enabling formation of interchain non-native disulphide bond, the first antigen-binding moiety can be stably expressed while in the meantime the antigen-binding capabilities of the antibody variable region are retained.

The TCR constant region comprising a mutated residue is also referred to herein as an “engineered” TCR constant region. In certain embodiments, the first TCR constant region (C1) of the polypeptide complex comprises an engineered TCR Alpha chain (CAlpha), and the second TCR constant region (C2) comprises an engineered TCR Beta chain (CBeta). In the polypeptide complexes provided herein, C1 comprises an engineered CBeta, and C2 comprises an engineered CAlpha.

In the polypeptide complexes provided herein, the engineered TCR constant region comprises one or more mutated cysteine residue within a contact interface of the first and/or the second engineered TCR constant regions.

The term “contact interface” as used herein refers to the particular region(s) on the polypeptides where the polypeptides interact/associate with each other. A contact interface comprises one or more amino acid residues that are capable of interacting with the corresponding amino acid residue(s) that comes into contact or association when interaction occurs. The amino acid residues in a contact interface may or may not be in a consecutive sequence. For example, when the interface is three-dimensional, the amino acid residues within the interface may be separated at different positions on the linear sequence.

In certain embodiments, one or more disulphide bonds can be formed between the engineered CAlpha and the engineered CBeta. In certain embodiments, the pair of cysteine residues are capable of forming a non-native interchain disulphide bond.

As used herein throughout the application, “XnY” with respect to a TCR constant region is intended to mean that the n^{th} amino acid residue X on the TCR constant region is replaced by amino acid residue Y, where X and Y are respectively the one-letter abbreviation of a particular amino acid residue.

In the polypeptide complexes provided herein, the engineered CBeta comprises or is SEQ ID NO: 121, and the engineered CAlpha comprises or is SEQ ID NO: 122.

The sequences represented by SEQ ID NO: 121 and SEQ ID NO: 122 are provided below:

SEQ ID NO: 121
LEDLKNVFPPEVAVFEPSEAEISHTQKATLVCLATGFYPDHVELSWWVNG
KEVHSGVCTDPQPLKEQPALQDSRYALSSRLRVSATFWQNPRNHFRCQVQ
FYGLSENDEWTQDRAKPVTQIVSAEA
SEQ ID NO: 122
PDIQNPDPAVYQLRDSKSSDKSVCLFTDFDSQTQVSQSKDSDVYITDKCV
LDMRSMDFKSNSAVAWSQKSDFACANAFQNSIIPEDTFFPSPESS

In the polypeptide complexes provided herein, one or more native glycosylation site present in the native TCR constant regions has been modified (e.g. removed) in the first antigen-binding moiety provided in the present disclosure. The term “glycosylation site” as used herein with respect to a polypeptide sequence refers to an amino acid residue with a side chain to which a carbohydrate moiety (e.g. an oligosaccharide structure) can be attached. Glycosylation of polypeptides like antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of native glycosylation sites can be conveniently accomplished by altering the amino acid sequence such that one or more of the above-described tripeptide sequences (for N-linked glycosylation sites) or one or more serine or threonine residues (for O-linked glycosylation sites) are substituted.

In the first antigen-binding moiety provided herein, at least one native glycosylation site is absent in the engineered TCR constant regions, for example, in the first and/or the second TCR constant regions. Without wishing to be bound by any theory, but it is believed that the first antigen-binding moiety provided herein can tolerate removal of all or part of the glycosylation sites without affecting the protein expression and stability, in contrast to existing teachings that presence of N-linked glycosylation sites on TCR constant region, such as CAlpha (i.e. N34, N68, and N79) and CBeta (i.e. N69) are necessary for protein expression and stability (see Wu et al., Mabs, 7:2, 364-376, 2015).

In the first antigen-binding moiety provided herein, the constant regions derived from a TCR are operably linked to the variable regions derived from an antibody.

In certain embodiments, the first antibody variable domain (VH) is fused to the first TCR constant region (C1) at a first conjunction domain, and the first antibody variable domain (VL) is fused to the second TCR constant region (C2) at a second conjunction domain.

“Conjunction domain” as used herein refers to a boundary or border region where two amino acid sequences are fused or combined. In certain embodiments, the first conjunction domain comprises at least a portion of the C terminal fragment of an antibody V/C conjunction, and the second conjunction domain comprises at least a portion of the N-terminal fragment of a TCR V/C conjunction.

The term “antibody V/C conjunction” as used herein refers to the boundary of antibody variable domain and constant domain, for example, the boundary between heavy chain variable domain and the CH1 domain, or between light chain variable domain and the light chain constant domain. Similarly, the term “TCR V/C conjunction” refers to the boundary of TCR variable domain and constant domain, for example, the boundary between TCR Alpha variable domain and constant domain, or between TCRBeta variable domain and constant domain.

In certain embodiments, the first polypeptide comprises a sequence comprising domains operably linked as in formula (I): VH-HCJ-C1, and the second polypeptide comprises a sequence comprising domains operably linked as in formula (II): VL-LCJ-C2, wherein:

VH is a heavy chain variable domain of an antibody;

HCJ is a first conjunction domain as defined supra;

C1 is a first TCR constant domain as defined supra;

VL is a light chain variable domain of an antibody;

LCJ is a second conjunction domain as defined supra;

C2 is a second TCR constant domain as defined supra.

Antibody Variable Region

The bispecific polypeptide complex provided herein comprises a first antigen-binding moiety associated with a second antigen-binding moiety, and one of them specifically binds to CD3, while the other specifically binds to CD20. In the polypeptide complex provided herein, the first antigen-binding moiety comprises a first heavy chain variable domain (VH1) and a first light chain variable domain (VL1) of a first antibody, and the second antigen-binding moiety comprises a second heavy chain variable domain (VH2) and a second light chain variable domain (VL2) of a second antibody, wherein the first antibody and the second antibody are different and are selected from the group consisting of an anti-CD3 antibody and an anti-CD20 antibody. In certain embodiments, the first antibody is an anti-CD3 antibody, and the second antibody is an anti-CD20 antibody. In certain other embodiments, the first antibody is an anti-CD20 antibody, and the second antibody is an anti-CD3 antibody.

In a conventional native antibody, a variable region comprises three CDR regions interposed by flanking framework (FR) regions, for example, as set forth in the following formula: FR1-CDR1-FR2-CDR2-FR3-CDR3-FR4, from N-terminus to C-terminus

a) Anti-CD3 Binding Moiety

In the polypeptide complex provided herein, the first antigen-binding moiety or the second antigen-binding moiety is an anti-CD3 binding moiety.

In certain embodiments, the anti-CD3 binding moiety is derived from the antibody W3278-T2U3.E17R-1.uIgG4. SP shown in Table A below. The CDR sequences of the anti-CD3 binding moiety of the W3278-T2U3.E17R-1.uIgG4. SP antibody are provided below.

TABLE A

| Antibody | | | | |
|---|---|---|---|---|
| ID: | | CDR1 | CDR2 | CDR3 |
| W3278- | VH | SEQ ID NO: 1 | SEQ ID NO: 2 | SEQ ID NO: 3 |
| EKFKG | | GYSFTTYYIH | WIFPGNDNIKYS | DSVSIYYFDY |
| T2U3.E17R- | VK | SEQ ID NO: 4 | SEQ ID NO: 5 | SEQ ID NO: 6 |
| 1.uIgG4.SP | | KSSQSLLNSRTRK | WASTRKS | TQSFILRT |
| | | NYLA | | |

Heavy and kappa light chain variable region sequences of the anti-CD3 binding moiety of the W3278-T2U3.E17R-1.uIgG4.SP antibody are provided below.

VH-Amino acid sequence (SEQ ID NO: 61):
QVQLVQSGAEVKKPGSSVKVSCKASGYSFTTYYIEWVRQAPGQGLEWMGWIFPGNDNI
KYSEKFKGRVTITADKSTSTAYMELSSLRSEDTAVYYCAIDSVSIYYFDYWGQGTLVTV
SS
VH-Nucleic acid sequence (SEQ ID NO: 101):
caggtgcaactcgtgcagtctggagctgaagtgaagaagcctgggtcttcagtcaaggtcagttgca
aggccagtgggtattccttcactacctactacatccactgggtgcggcaggcaccaggacaggggct
tgagtggatgggctggatctttcccggcaacgataatattaagtacagcgagaagttcaaagggagg
gtcaccattaccgccgacaaatccacttccacagcctacatggagttgagcagcctgagatccgagg
atacagccgtgtactactgtgccattgacagcgtgtccatctactactttgactactggggccaggg
cacactggtcacagtgagcagc
VK-Amino acid sequence (SEQ ID NO: 62):
DIVMTQSPDSLAVSLGERATINCKSSQSLLNSRTRKNYLAWYQQKPGQPPKLLIYWAST
RKSGVPDRFSGSGSGTDFTLTISSLQAEDVAVYYCTQSFILRTFGGGTKVEIK
VK-Nucleic acid sequence (SEQ ID NO: 102):
gacatcgtcatgacccagtccccagactctttggcagtgtctctcggggaaagagctaccatcaact
gcaagagcagccagtcccttctgaacagcaggaccaggaagaattacctcgcctggtaccaacagaa
gcccggacagcctcctaagctcctgatctactgggcctcaacccggaagagtggagtgcccgatcgc
tttagcgggagcggctccgggacagatttcacactgacaatttcctccctgcaggccgaggacgtcg
ccgtgtattactgtactcagagcttcattctgcggacatttggcggcgggactaaagtggagattaag

In certain embodiments, the anti-CD3 binding moiety is derived from the antibody W3278-T3U2.F16-1.uIgG4.SP shown in Table B below. The CDR sequences of the anti-CD3 binding moiety of the W3278-T3U2.F16-1.uIgG4.SP antibody are provided below.

TABLE B

Antibody

| | | | |
|---|---|---|---|
| ID: | CDR1 | CDR2 | CDR3 |

| | | | | |
|---|---|---|---|---|
| W3278- | VH | SEQ ID NO: 13 | SEQ ID NO: 14 | SEQ ID |
| T3U2.F16- | | GFAFTDYYIEI | WISPGNVNTKY | NO: 15 |
| 1.uIgG4.SP | | | NENFKG | DGYSLY |
| | | | | YFDY |
| | VK | SEQ ID NO: 16 | SEQ ID NO: 17 | SEQ ID |
| | | KSSQSLLNSRTRK | WASTRQS | NO: 18 |
| | | NYLA | | TQSHTLRT |

Heavy and kappa light chain variable region sequences of the anti-CD3 binding moiety of the W3278-T3U2.F16-1.uIgG4. SP antibody are provided below.

VH-Amino acid sequence (SEQ ID NO: 63):
QVQLVQSGAEVKKPGSSVKVSCKASGFAFTDYYIHWVRQAPGQGLEWMGWISPGNVN
TKYNENFKGRVTITADKSTSTAYMELSSLRSEDTAVYYCARDGYSLYYFDYWGQGTLV
TVSS
VH-Nucleic acid sequence (SEQ ID NO: 103):
caggtgcagcttgtgcagtctggggcagaagtgaagaagcctgggtctagtgtcaaggtgtcatgca
aggctagcgggttcgcctttactgactactacatccactgggtgcggcaggctcccggacaagggtt
ggagtggatgggatggatctccccaggcaatgtcaacacaaagtacaacgagaacttcaaaggccgc
gtcaccattaccgccgacaagagcacctccacagcctacatggagctgtccagcctcagaagcgagg
acactgccgtctactactgtgccagggatgggtactccctgtattactttgattactggggccaggg
cacactggtgacagtgagctcc
VK-Amino acid sequence (SEQ ID NO: 64):
DIVMTQSPDSLAVSLGERATINCKSSQSLLNSRTRKNYLAWYQQKPGQPPKLLIYWAST
RQSGVPDRFSGSGSGTDFTLTISSLQAEDVAVYYCTQSHTLRTFGGGTKVEIK
VK-Nucleic acid sequence (SEQ ID NO: 104):
gatatcgtgatgacccagagcccagactcccttgctgtctccctcggcgaaagagcaaccatcaact
gcaagagctcccaaagcctgctgaactccaggaccaggaagaattacctggcctggtatcagcagaa
gcccggccagcctcctaagctgctcatctactgggcctccacccggcagtctggggtgcccgatcgg
tttagtggatctgggagcgggacagacttcacattgacaattagctcactgcaggccgaggacgtgg
ccgtctactactgtactcagagccacactetccgcacattcggcggagggactaaagtggagattaag

In certain embodiments, the anti-CD3 binding moiety is derived from the antibody W3278-U2T3.F18R-1.uIgG4.SP shown in Table C below. The CDR sequences of the anti-CD3 binding moiety of the W3278-U2T3.F18R-1.uIgG4.SP antibody are provided below.

TABLE C

| Antibody | | | | |
|---|---|---|---|---|
| ID: | CDR1 | CDR2 | CDR3 | |
| W3278- | VH | SEQ ID NO: 25 | SEQ ID NO: 26 | SEQ ID |
| U2T3.F18R- | | GFAFTDYYIH | WISPGNVNTKY | NO: 27 |
| 1.uIgG4.SP | | | NENFKG | DGYSLYYFDY |
| | VK | SEQ ID NO: 28 | SEQ ID NO: 29 | SEQ ID |
| | | KSSQSLLNSRTRK | WASTRQS | NO: 30 |
| | | NYLA | | TQSHTLRT |

Heavy and kappa light chain variable region sequences of the anti-CD3 binding moiety of the W3278-U2T3.F18R-1.uIgG4.SP antibody are provided below.

VH-Amino acid sequence (SEQ ID NO: 65):
QVQLVQSGAEVKKPGSSVKVSCKASGFAFTDYYIHWVRQAPGQGLEWMGWISPGNVN
TKYNENFKGRVTITADKSTSTAYMELSSLRSEDTAVYYCARDGYSLYYFDYWGQGTLV
TVSS
VII-Nucleic acid sequence (SEQ ID NO: 105):
caggtgcagcttgtgcagtctggggcagaagtgaagaagcctgggtctagtgtcaaggtgtcatg
caaggctagcgggttcgcctttactgactactacatccactgggtgcggcaggctcccggacaag
ggttggagtggatgggatggatctccccaggcaatgtcaacacaaagtacaacgagaacttcaaa
ggccgcgtcaccattaccgccgacaagagcacctccacagcctacatggagctgtccagcctcag
aagcgaggacactgccgtctactactgtgccagggatgggtactccctgtattactttgattact
ggggccagggcacactggtgacagtgagctcc
VK-Amino acid sequence (SEQ ID NO: 66):
DIVMTQSPDSLAVSLGERATINCKSSQSLLNSRTRKNYLAWYQQKPGQPPKLLIYWAST
RQSGVPDRFSGSGSGTDFTLTISSLQAEDVAVYYCTQSHTLRTFGGGTKVEIK
VK-Nucleic acid sequence (SEQ ID NO: 106):
gatatcgtgatgacccagagcccagactcccttgctgtctccctcggcgaaagagcaaccatcaa
ctgcaagagctcccaaagcctgctgaactccaggaccaggaagaattacctggcctggtatcagc
agaagcccggccagcctcctaagctgctcatctactgggcctccacccggcagtctggggtgccc
gatcggtttagtggatctgggagcgggacagacttcacattgacaattagctcactgcaggccga
ggacgtggccgtctactactgtactcagagccacactctccgcacattcggcggagggactaaag
tggagattaag

In certain embodiments, the anti-CD3 binding moiety is derived from the antibody W3278-U3T2.F18R-1.uIgG4.SP shown in Table D below. The CDR sequences of the anti-CD3 binding moiety of the W3278-U3T2.F18R-1.uIgG4.SP antibody are provided below.

TABLE D

| Antibody | | | | |
|---|---|---|---|---|
| ID: | | CDR1 | CDR2 | CDR3 |
| W3278- | VH | SEQ ID | SEQ ID | SEQ ID |
| U3T2.F18R- | | NO: 37 | NO: 38 | NO: 39 |
| 1.uIgG4.SP | | GYSFTT | WIFPGN | DSVSIY |
| | | YYIH | DNIKYS | YFDY |
| | | | EKFKG | |
| | VK | SEQ ID | SEQ ID | SEQ ID |
| | | NO: 40 | NO: 41 | NO: 42 |
| | | KSSQSLL | WASTRKS | TQSFILRT |
| | | NSRTRK | | |
| | | NYLA | | |

Heavy and kappa light chain variable region sequences of the anti-CD3 binding moiety of the W3278-U3T2.F18R-1.uIgG4.SP antibody are provided below.

VH-Amino acid sequence
(SEQ ID NO: 67):
QVQLVQSGAEVKKPGSSVKVSCKASGYSFTTYYIH
WVRQAPGQGLEWMGWIFPGNDNIKYSEKFKGRVTI
TADKSTSTAYMELSSLRSEDTAVYYCAIDSVSIYY
FDYWGQGTLVTVSS
VH-Nucleic acid sequence
(SEQ ID NO: 107):
caggtgcaactcgtgcagtctggagctgaagtgaa
gaagcctgggtcttcagtcaaggtcagttgcaagg
ccagtgggtattccttcactacctactacatccac
tgggtgcggcaggcaccaggacaggggcttgagtg
gatgggctggatctttcccggcaacgataatatta
agtacagcgagaagttcaaagggagggtcaccatt
accgccgacaaatccacttccacagcctacatgga
gttgagcagcctgagatccgaggatacagccgtgt
actactgtgccattgacagcgtgtccatctactac
tttgactactggggccagggcacactggtcacagt
gagcagc
VK-Amino acid sequence
(SEQ ID NO: 68):
DIVMTQSPDSLAVSLGERATINCKSSQSLLNSRTR
KNYLAWYQQKPGQPPKLLIYWASTRKSGVPDRFSG
SGSGTDFTLTISSLQAEDVAVYYCTQSFILRTFGG
GTKVEIK
VK-Nucleic acid sequence
(SEQ ID NO: 108):
gacatcgtcatgacccagtccccagactctttggc
agtgtctctcggggaaagagctaccatcaactgca
agagcagccagtcccttctgaacagcaggaccagg
aagaattacctcgcctggtaccaacagaagcccgg
acagcctcctaagctcctgatctactgggcctcaa
cccggaagagtggagtgcccgatcgctttagcggg
agcggctccgggacagatttcacactgacaatttc
ctccctgcaggccgaggacgtcgccgtgtattact
gtactcagagcttcattctgcggacatttggcggc
gggactaaagtggagattaag

In certain embodiments, the anti-CD3 binding moiety is derived from the antibody W3278-T3U2.F17R-1.uIgG4.SP shown in Table E below. The CDR sequences of the anti-CD3 binding moiety of the W3278-T3U2.F17R-1.uIgG4.SP antibody are provided below.

TABLE E

| Antibody | | | | |
|---|---|---|---|---|
| ID: | | CDR1 | CDR2 | CDR3 |
| W3278- | VH | SEQ ID | SEQ ID | SEQ ID |
| T3U2.F17R- | | NO: 49 | NO: 50 | NO: 51 |
| 1.uIgG4.SP | | GFAFTD | WISPGN | DGYSLY |
| | | YYIH | VNTKY | YFDY |
| | | | NENFKG | |
| | VK | SEQ ID | SEQ ID | SEQ ID |
| | | NO: 52 | NO: 53 | NO: 54 |
| | | KSSQSLL | WAST | TQSHT |
| | | NSRTRK | RQS | LRT |
| | | NYLA | | |

Heavy and kappa light chain variable region sequences of the anti-CD3 binding moiety of the W3278-T3U2.F17R-1.uIgG4.SP antibody are provided below.

VH-Amino acid sequence
(SEQ ID NO: 69):
QVQLVQSGAEVKKPGSSVKVSCKASGFAFTDYYIH
WVRQAPGQGLEWMGWISPGNVNTKYNENFKGRVTI
TADKSTSTAYMELSSLRSEDTAVYYCARDGYSLYY
FDYWGQGTLVTVSS
VH-Nucleic acid sequence
(SEQ ID NO: 109):
caggtgcagcttgtgcagtctggggcagaagtgaa
gaagcctgggtctagtgtcaaggtgtcatgcaagg
ctagcgggttcgcctttactgactactacatccac
tgggtgcggcaggctcccggacaagggttggagtg
gatgggatggatctccccaggcaatgtcaacacaa
agtacaacgagaacttcaaaggccgcgtcaccatt
accgccgacaagagcacctccacagcctacatgga
gctgtccagcctcagaagcgaggacactgccgtct
actactgtgccagggatgggtactccctgtattac
tttgattactggggccagggcacactggtgacagt
gagctcc
VK-Amino acid sequence
(SEQ ID NO: 70):
DIVMTQSPDSLAVSLGERATINCKSSQSLLNSRTR
KNYLAWYQQKPGQPPKLLIYWASTRQSGVPDRFSG
SGSGTDFTLTISSLQAEDVAVYYCTQSHTLRTFGG
GTKVEIK
VK-Nucleic acid sequence
(SEQ ID NO: 110):
gatatcgtgatgacccagagcccagactcccttgc
tgtctccctcggcgaaagagcaaccatcaactgca
agagctcccaaagcctgctgaactccaggaccagg
aagaattacctggcctggtatcagcagaagcccgg
ccagcctcctaagctgctcatctactgggcctcca
cccggcagtctggggtgcccgatcggtttagtgga
tctgggagcgggacagacttcacattgacaattag
ctcactgcaggccgaggacgtggccgtctactact
gtactcagagccacactctccgcacattcggcgga
gggactaaagtggagattaag

The anti-CD3 binding moiety provided herein further comprises suitable framework region (FR) sequences, as long as the anti-CD3 binding moiety can specifically bind to CD3.

The anti-CD3 antibodies of the present disclosure have the specific binding affinity to CD3-expressing cell (e.g. CD4 T cell) and can activate human T cells and trigger cytokine release of TNFalpha and IFNgamma.

Binding affinity of the anti-CD3 binding moiety provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g. K_{D}) can be appropriately determined using suitable methods known in the art, including, for example, flow cytometry assay. In some embodiments, binding of the antibody to the antigen at different concentrations can be determined by flow cytometry, the determined mean fluorescence intensity (MFI) can be firstly plotted against antibody concentration, K_{D} value can then be calculated by fitting the dependence of specific binding fluorescence intensity (Y) and the concentration of antibodies (X) into the one site saturation equation: Y=Bₘₐₓ*X/(K_{D}+X) using Prism version 5 (GraphPad Software, San Diego, Calif.), wherein Bₘₐₓ refers to the maximum specific binding of the tested antibody to the antigen.

In certain embodiments, the anti-CD3 binding moiety provided herein is capable of specifically binding to human CD3 expressed on a cell surface, or a recombinant human CD3. CD3 is a receptor expressed on cell. A recombinant CD3 is soluble CD3 which is recombinantly expressed and is not associated with a cell membrane. A recombinant CD3 can be prepared by various recombinant technologies. In one example, the CD3 DNA sequence encoding the extracellular domain of human CD3 (NP 000724.1) (Met1-Asp126) can be fused with a polyhistidine tag at the C-terminus in an expression vector, and then transfected and expressed in 293E cells and purified by Ni-Affinity chromatography.

In some embodiments, the anti-CD3 binding moiety provided herein is capable of specifically binding to human CD3 expressed on surface of cells with a binding affinity (K_{D}) of no more than 5×10⁻⁹ M, no more than 4×10⁻⁹ M, no more than 3×10⁻⁹ M, no more than 2×10⁻⁹ M, no more than 10⁻⁹ M, no more than 5×10⁻¹⁰ M, no more than 4×10⁻¹⁰ M, no more than 3×10⁻¹⁰ M, no more than 2×10⁻¹⁰ M, no more than 10⁻¹⁰ M no more than 5×10⁻¹¹ M, or no more than 4×10⁻¹¹ M, no more than 3×10⁻¹¹ M, or no more than 2×10⁻¹¹ M, or no more than 10⁻¹¹ M as measured by flow cytometry assay.

In certain embodiments, the anti-CD3 binding moiety provided herein cross-reacts with cynomolgus monkey CD3, for example, cynomolgus monkey CD3 expressed on a cell surface, or a soluble recombinant cynomolgus monkey CD3.

Binding of the anti-CD3 binding moiety to recombinant CD3 or CD3 expressed on surface of cells can be measured by methods known in the art, for example, sandwich assay such as ELISA, Western Blot, flow cytometry assay, and other binding assay. In certain embodiments, the anti-CD3 binding moiety provided herein specifically bind to recombinant human CD3 at an EC₅₀ (i.e. 50% binding concentration) of no more than 0.01 nM, no more than 0.02 nM, no more than 0.03 nM, no more than 0.04 nM, no more than 0.05 nM, no more than 0.06 nM, no more than 0.07 nM or no more than 0.08 nM by ELISA. In certain embodiments, the anti-CD3 binding moiety provided herein specifically bind to human CD3 expressed on surface of cells at an EC₅₀ of no more than 0.5 nM, no more than 0.6 nM, no more than 0.7 nM, no more than 0.8 nM, no more than 0.9 nM, no more than 1 nM, no more than 2 nM, no more than 3 nM, no more than 4 nM, no more than 5 nM, no more than 6 nM, no more than 7 nM, no more than 8 nM, no more than 9 nM or no more than 10 nM by flow cytometry assay.

In certain embodiments, the anti-CD3 binding moiety binds to cynomolgus monkey CD3 with a binding affinity similar to that of human CD3.

In certain embodiments, the anti-CD3 binding moiety provided herein specifically binds to recombinant cynomolgus monkey CD3 with an EC₅₀ of no more than 0.001 nM, no more than 0.005 nM, no more than 0.01 nM, no more than 0.02 nM, no more than 0.03 nM, no more than 0.04 nM, or no more than 0.05 nM by ELISA.

In certain embodiments, the anti-CD3 binding moiety provided herein has a specific binding affinity to human CD3 which is sufficient to provide for diagnostic and/or therapeutic use.

A number of therapeutic strategies modulate T cell immunity by targeting TCR signaling, particularly by anti-human CD3 monoclonal antibodies that are clinically used.

b) Anti-CD20 Antibody

In the polypeptide complex provided herein, the first antigen-binding moiety or the second antigen-binding moiety is an anti-CD20 binding moiety.

In certain embodiments, the anti-CD20 binding moiety is derived from the antibody W3278-T2U3.E17R-1.uIgG4.SP shown in Table A′ below. The CDR sequences of the anti-CD20 binding moiety of the W3278-T2U3.E17R-1.uIgG4.SP antibody are provided below.

TABLE A′

| Antibody | | | | |
|---|---|---|---|---|
| ID: | | CDR1 | CDR2 | CDR3 |
| W3278- | VH | SEQ ID | SEQ ID | SEQ ID |
| T2U3.E17R- | | NO: 7 | NO: 8 | NO: 9 |
| 1.uIgG4.SP | | GFTFND | TISWNS | DIQYG |
| | | YAMH | GSIGYA | NYYYG |
| | | | DSVKG | MDV |
| | VK | SEQ ID | SEQ ID | SEQ ID |
| | | NO: 10 | NO: 11 | NO: 12 |
| | | RASQSV | DASNR | QQRSN |
| | | SSYLA | AT | WPIT |

Heavy and kappa light chain variable region sequences of the anti-CD20 binding moiety of the W3278-T2U3.E17R-1.uIgG4.SP antibody are provided below.

VH-Amino acid sequence
(SEQ ID NO: 71):
EVQLVESGGGLVQPGRSLRLSCAASGFTFNDYAMH
WVRQAPGKGLEWVSTISWNSGSIGYADSVKGRFTI
SRDNAKKSLYLQMNSLRAEDTALYYCAKDIQYGNY
YYGMDVWGQGTTVTVSS
VH-Nucleic acid sequence
(SEQ ID NO: 111):
gaggtgcaattggtggagagcggaggagggctcgt
gcagcctggaagatctcttaggctgagttgcgctg
catctgggttcacattcaacgactacgccatgcac
tgggtgaggcaggctcccggcaaagggctggaatg
ggtgtcaactatctcctggaactccggcagcatcg
gctacgccgatagcgtcaagggccggtttacaatt
tcccgcgataacgccaagaagtccctgtacctgca
gatgaacagcctgcgggccgaggatactgccctct
actactgtgccaaggacattcagtacgggaattac
tattacgggatggacgtctggggccaggggaccac
cgtgacagtcagctcc
VK-Amino acid sequence
(SEQ ID NO: 72):
EIVLTQSPATLSLSPGERATLSCRASQSVSSYLAW
YQQKPGQAPRLLIYDASNRATGIPARFSGSGSGTD
FTLTISSLEPEDFAVYYCQQRSNWPITFGQGTRLE
IK
VK-Nucleic acid sequence
(SEQ ID NO: 112):
gaaatcgtgctgacccagtccccagcaaccctctc
cctttctcctggagagagagctaccctcagctgta
gggcctcacagtctgtctccagttacctggcttgg
taccagcagaaacccgggcaggcccctaggttgct
gatctacgacgccagcaatagggccactggcatcc
cagcccggttttccggaagcggcagcgggacagat
ttcacactcactattagcagcctggagcccgagga
cttcgccgtgtactattgccagcagcggtccaact
ggcccattacatttggccaagggacacgcctggag
attaag

In certain embodiments, the anti-CD20 binding moiety is derived from the antibody W3278-T3U2.F16-1.uIgG4.SP shown in Table B′ below. The CDR sequences of the anti-CD20 binding moiety of the W3278-T3U2.F16-1.uIgG4. SP antibody are provided below.

TABLE B′

| Antibody ID: | | CDR1 | CDR2 | CDR3 |
|---|---|---|---|---|
| W3278-T3U2.F16- | VH | SEQ ID | SEQ ID | SEQ ID |
| 1.uIgG4.SP | | NO: 19 | NO: 20 | NO: 21 |
| | | GYTFT | AIYPGN | STYYGG |
| | | SYNMH | GDTSY | DWYF |
| | | | NQKFKG | NV |
| | VK | SEQ ID | SEQ ID | SEQ ID |
| | | NO: 22 | NO: 23 | NO: 24 |
| | | RASSSV | ATSNLAS | QQWTS |
| | | SYIH | | NPPT |

Heavy and kappa light chain variable region sequences of the anti-CD20 binding moiety of the W3278-T3U2.F16-1.uIgG4. SP antibody are provided below.

VH-Amino acid sequence
(SEQ ID NO: 73):
QVQLQQPGAELVKPGASVKMSCKASGYTFTSYNMH
WVKQTPGRGLEWIGAIYPGNGDTSYNQKFKGKATL
TADKSSSTAYMQLSSLTSEDSAVYYCARSTYYGGD
WYFNVWGAGTTVTVSA
VH-Nucleic acid sequence
(SEQ ID NO: 113):
caggtccagctgcagcagcccggagccgaactggt
caaacccggggctagcgtgaaaatgtcttgcaaag
caagtggttacacattcacttcctataacatgcac
tgggtgaagcagacacctgggcgaggtctggaatg
gatcggcgccatctacccaggcaacggagacacta
gctataatcagaagtttaaaggaaaggccaccctg
acagctgataagtccagctctaccgcttacatgca
gctgagttcactgacaagtgaggactcagcagtgt
actattgcgcccgttctacctactatggcggagat
tggtatttcaatgtgtggggcgccggtaccacagt
caccgtgtccgcc
VK-Amino acid sequence
(SEQ ID NO: 74):
QIVLSQSPAILSASPGEKVTMTCRASSSVSYIHWF
QQKPGSSPKPWIYATSNLASGVPVRFSGSGSGTSY
SLTISRVEAEDAATYYCQQWTSNPPTFGGGTKLEI
K
VK-Nucleic acid sequence
(SEQ ID NO: 114):
cagattgtcctgagccagagccctgccatcctgtc
tgctagtcccggcgagaaggtgaccatgacatgca
gggcatccagctctgtctcctacatccactggttc
cagcagaagcccgggagttcacctaaaccatggat
ctacgctacatccaacctggcaagcggtgtgcctg
tcaggttttcaggttccggcagcggaacatcttac
agtctgactatttctcgggtggaggccgaagacgc
cgctacctactattgccagcagtggacctccaatc
cccctacattcggcggagggactaagctggagatc
aaa

In certain embodiments, the anti-CD20 binding moiety is derived from the antibody W3278-U2T3.F18R-1.uIgG4.SP shown in Table C′ below. The CDR sequences of the anti-CD20 binding moiety of the W3278-U2T3.F18R-1.uIgG4.SP antibody are provided below.

TABLE C′

| Antibody ID: | | CDR1 | CDR2 | CDR3 |
|---|---|---|---|---|
| W3278- | VH | SEQ ID | SEQ ID | SEQ ID |
| U2T3.F18R- | | NO: 31 | NO: 32 | NO: 33 |
| l.uIgG4.SP | | GYTFT | AIYPG | STYYG |
| | | SYNMH | NGDTSY | GDWYF |
| | | | NQKFKG | NV |
| | VK | SEQ ID | SEQ ID | SEQ ID |
| | | NO: 34 | NO: 35 | NO: 36 |
| | | RASSS | ATSNL | QQWTS |
| | | VSYIH | AS | NPPT |

Heavy and kappa light chain variable region sequences of the anti-CD20 binding moiety of the W3278-U2T3.F18R-1.uIgG4.SP antibody are provided below.

VH-Amino acid sequence
(SEQ ID NO: 75):
QVQLQQPGAELVKPGASVKMSCKASGYTFTSYNMH
WVKQTPGRGLEWIGAIYPGNGDTSYNQKFKGKATL
TADKSSSTAYMQLSSLTSEDSAVYYCARSTYYGGD
WYFNVWGAGTTVTVSA
VH-Nucleic acid sequence
(SEQ ID NO: 115):
caggtccagctgcagcagcccggagccgaactggt
caaacccggggctagcgtgaaaatgtcttgcaaag
caagtggttacacattcacttcctataacatgcac
tgggtgaagcagacacctgggcgaggtctggaatg
gatcggcgccatctacccaggcaacggagacacta
gctataatcagaagtttaaaggaaaggccaccctg
acagctgataagtccagctctaccgcttacatgca
gctgagttcactgacaagtgaggactcagcagtgt
actattgcgcccgttctacctactatggcggagat
tggtatttcaatgtgtggggcgccggtaccacagt
caccgtgtccgcc
VK-Amino acid sequence
(SEQ ID NO: 76):
QIVLSQSPAILSASPGEKVTMTCRASSSVSYIHWF
QQKPGSSPKPWIYATSNLASGVPVRJFSGSGSGTS
YSLTISRVEAEDAATYYCQQWTSNPPTFGGGTKLE
IK
VK-Nucleic acid sequence
(SEQ ID NO: 116):
cagattgtcctgagccagagccctgccatcctgtc
tgctagtcccggcgagaaggtgaccatgacatgca
gggcatccagctctgtctcctacatccactggttc
cagcagaagcccgggagttcacctaaaccatggat
ctacgctacatccaacctggcaagcggtgtgcctg
tcaggttttcaggttccggcagcggaacatcttac
agtctgactatttctcgggtggaggccgaagacgc
cgctacctactattgccagcagtggacctccaatc
cccctacattcggcggagggactaagctggagatc
aaa

In certain embodiments, the anti-CD20 binding moiety is derived from the antibody W3278-U3T2.F18R-1.uIgG4.SP shown in Table D′ below. The CDR sequences of the anti-CD20 binding moiety of the W3278-U3T2.F18R-1.uIgG4.SP antibody are provided below.

TABLE D′

| Antibody ID: | | CDR1 | CDR2 | CDR3 |
|---|---|---|---|---|
| W3278- | VH | SEQ ID | SEQ ID | SEQ ID |
| U3T2.F18R- | | NO: 43 | NO: 44 | NO: 45 |
| 1.uIgG4.SP | | GFTFND | TISWNS | DIQYGN |
| | | YAMH | GSIGYA | YYYG |
| | | | DSVKG | MDV |
| | VK | SEQ ID | SEQ ID | SEQ ID |
| | | NO: 46 | NO: 47 | NO: 48 |
| | | RASQSV | DASNR | QQRSN |
| | | SSYLA | AT | WPIT |

Heavy and kappa light chain variable region sequences of the anti-CD20 binding moiety of the W3278-U3T2.F18R-1.uIgG4.SP antibody are provided below.

VH-Amino acid sequence
(SEQ ID NO: 77):
EVQLVESGGGLVQPGRSLRLSCAASGFTFNDYAMH
WVRQAPGKGLEWVSTISWNSGSIGYADSVKGRFTI
SRDNAKKSLYLQMNSLRAEDTALYYCAKDIQYGNY
YYGMDVWGQGTTVTVSS
VH-Nucleic acid sequence
(SEQ ID NO: 117):
gaggtgcaattggtggagagcggaggagggctcgt
gcagcctggaagatctcttaggctgagttgcgctg
catctgggttcacattcaacgactacgccatgcac
tgggtgaggcaggctcccggcaaagggctggaatg
ggtgtcaactatctcctggaactccggcagcatcg
gctacgccgatagcgtcaagggccggtttacaatt
tcccgcgataacgccaagaagtccctgtacctgca
gatgaacagcctgcgggccgaggatactgccctct
actactgtgccaaggacattcagtacgggaattac
tattacgggatggacgtctggggccaggggaccac
cgtgacagtcagctcc
VK-Amino acid sequence
(SEQ ID NO: 78):
EIVLTQSPATLSLSPGERATLSCRASQSVSSYLAW
YQQKPGQAPRLLIYDASNRATGIPARFSGSGSGTD
FTLTISSLEPEDFAVYYCQQRSNWPITFGQGTRLE
IK
VK-Nucleic acid sequence
(SEQ ID NO: 118):
gaaatcgtgctgacccagtccccagcaaccctctc
cctttctcctggagagagagctaccctcagctgta
gggcctcacagtctgtctccagttacctggcttgg
taccagcagaaacccgggcaggcccctaggttgct
gatctacgacgccagcaatagggccactggcatcc
cagcccggttttccggaagcggcagcgggacagat
ttcacactcactattagcagcctggagcccgagga
cttcgccgtgtactattgccagcagcggtccaact
ggcccattacatttggccaagggacacgcctggag
attaag

In certain embodiments, the anti-CD20 binding moiety is derived from the antibody W3278-T3U2.F17R-1.uIgG4.SP shown in Table E′ below. The CDR sequences of the anti-CD20 binding moiety of the W3278-T3U2.F17R-1.uIgG4.SP antibody are provided below.

TABLE E′

| Antibody | | | | |
|---|---|---|---|---|
| ID: | | CDR1 | CDR2 | CDR3 |
| W3278- | VH | SEQ ID | SEQ ID | SEQ ID |
| T3U2.F17R- | | NO: 55 | NO: 56 | NO: 57 |
| 1.uIgG4.SP | | GYTFT | AIYPGN | STYYGG |
| | | SYNMH | GDTSY | DWYF |
| | | | NQKFKG | NV |
| | VK | SEQ ID | SEQ ID | SEQ ID |
| | | NO: 58 | NO: 59 | NO: 60 |
| | | RASSS | ATSNL | QQWTSN |
| | | VSYIH | AS | PPT |

Heavy and kappa light chain variable region sequences of the anti-CD20 binding moiety of the W3278-T3U2.F17R-1.uIgG4.SP antibody are provided below.

VH-Amino acid sequence
(SEQ ID NO: 79):
QVQLQQPGAELVKPGASVKMSCKASGYTFTSYNMH
WVKQTPGRGLEWIGAIYPGNGDTSYNQKFKGKATL
TADKSSSTAYMQLSSLTSEDSAVYYCARSTYYGGD
WYFNVWGAGTTVTVSA
VH-Nucleic acid sequence
(SEQ ID NO: 119):
caggtccagctgcagcagcccggagccgaactggt
caaacccggggctagcgtgaaaatgtcttgcaaag
caagtggttacacattcacttcctataacatgcac
tgggtgaagcagacacctgggcgaggtctggaatg
gatcggcgccatctacccaggcaacggagacacta
gctataatcagaagtttaaaggaaaggccaccctg
acagctgataagtccagctctaccgcttacatgca
gctgagttcactgacaagtgaggactcagcagtgt
actattgcgcccgttctacctactatggcggagat
tggtatttcaatgtgtggggcgccggtaccacagt
caccgtgtccgcc
VK-Amino acid sequence
(SEQ ID NO: 80):
QIVLSQSPAILSASPGEKVTMTCRASSSVSYIHWF
QQKPGSSPKPWIYATSNLASGVPVRFSGSGSGTSY
SLTISRVEAEDAATYYCQQWTSNPPTFGGGTKLEI
KVK-Nucleic acid sequence
(SEQ ID NO: 120):
cagattgtcctgagccagagccctgccatcctgtc
tgctagtcccggcgagaaggtgaccatgacatgca
gggcatccagctctgtctcctacatccactggttc
cagcagaagcccgggagttcacctaaaccatggat
ctacgctacatccaacctggcaagcggtgtgcctg
tcaggttttcaggttccggcagcggaacatcttac
agtctgactatttctcgggtggaggccgaagacgc
cgctacctactattgccagcagtggacctccaatc
cccctacattcggcggagggactaagctggagatc
aaa

The anti-CD20 binding moiety provided herein further comprises suitable framework region (FR) sequences, as long as the anti-CD20 binding moiety can specifically bind to CD20.

In some embodiments, the anti-CD20 binding moiety provided herein is capable of specifically binding to human CD20 expressed on surface of cells with a binding affinity (K_{D}) of no more than 5×10⁻⁹ M, no more than 1×10⁻⁹ M, no more than 9×10⁻¹⁰ M, no more than 8×10⁻¹⁰ M, no more than 7×10⁻¹⁰ M, no more than 6×10⁻¹¹ M no more than 5×10⁻¹⁰ M, no more than 4×10¹⁰ M, no more than 3×10⁻¹⁰ M no more than 2×10⁻¹⁰ M, or no more than 1×10⁻¹⁰ M as measured by flow cytometry assay.

In certain embodiments, the anti-CD20 binding moiety provided herein cross-reacts with cynomolgus monkey CD20, for example, cynomolgus monkey CD20 expressed on a cell surface, or a soluble recombinant cynomolgus monkey CD20.

Binding of the anti-CD20 binding moiety to CD20 expressed on a cell can be measured by methods known in the art, for example, sandwich assay such as ELISA, Western Blot, flow cytometry assay, and other binding assays. In certain embodiments, the anti-CD20 binding moiety provided herein specifically binds to human CD20 expressed on a cell with an EC₅₀ of no more than 0.01 nM, no more than 0.02 nM, no more than 0.03 nM, no more than 0.04 nM, no more than 0.05 nM, no more than 0.1 nM, no more than 0.2 nM, no more than 0.3 nM, no more than 0.4 nM, no more than 0.5 nM, no more than 0.6 nM, no more than 0.7 nM, no more than 0.8 nM, no more than 0.9 nM, or no more than 1 nM by flow cytometry assay.

In certain embodiments, the anti-CD20 binding moiety binds to cynomolgus monkey CD20 with a binding affinity similar to that of human CD20. In certain embodiments, the anti-CD20 binding moiety provided herein specifically binds to cynomolgus monkey CD20 expressed on a cell at an EC₅₀ of no more than 0.2 nM, no more than 0.5 nM, no more than 0.8 nM, no more than 1 nM, no more than 2 nM, or no more than 3 nM by flow cytometry assay.

In certain embodiments, the anti-CD20 binding moiety provided herein is internalized by a CD20-expressing cell at an EC₅₀ of no more than 1 pM, no more than 2 pM, no more than 3 pM, no more than 4 pM, no more than 5 pM, no more than 6 pM, no more than 7 pM, no more than 8 pM, no more than 9 pM, no more than 10 pM, no more than 11 pM, no more than 12 pM, no more than 13 pM, no more than 14 pM, no more than 15 pM, no more than 16 pM, no more than 17 pM, no more than 18 pM, no more than 19 pM, no more than 20 pM, no more than 21 pM, no more than 22 pM, no more than 23 pM, no more than 24 pM, no more than 25 pM, no more than 30 pM, no more than 35 pM, no more than 40 pM, no more than 45 pM, or no more than 50 pM by Fab-Zap assay.

Bispecific Polypeptide Complex

In certain embodiments, the first and/or the second antigen binding moiety is multivalent, such as bivalent, trivalent, tetravalent. The term “valent” as used herein refers to the presence of a specified number of antigen binding sites in a given molecule. As such, the terms “bivalent”, “tetravalent”, and “hexavalent” denote the presence of two binding sites, four binding sites, and six binding sites, respectively, in an antigen-binding molecule. A bivalent molecule can be monospecific if the two binding sites are both for specific binding of the same antigen or the same epitope. Similarly, a trivalent molecule can be bispecific, for example, when two binding sites are monospecific for a first antigen (or epitope) and the third binding site is specific for a second antigen (or epitope). In certain embodiments, the first and/or the second antigen-binding moiety in the bispecific polypeptide complex provided herein can be bivalent, trivalent, or tetravalent, with at least two binding sites specific for the same antigen or epitope. This, in certain embodiments, provides for stronger binding to the antigen or the epitope than a monovalent counterpart. In certain embodiments, in a bivalent antigen-binding moiety, the first valent of binding site and the second valent of binding site are structurally identical (i.e. having the same sequences), or structurally different (i.e. having different sequences albeit with the same specificity).

In certain embodiments, the first and/or the second antigen binding moiety is multivalent and comprises two or more antigen binding sites operably linked together, with or without a spacer.

In certain embodiments, the second antigen binding moiety comprises two or more Fab of the second antibody. The two Fabs can be operably linked to each other, for example the first Fab can be covalently attached to the second Fab via heavy chain, with or without a spacer in between.

In certain embodiments, the first antigen-binding moiety is linked to a first dimerization domain, and the second antigen-binding moiety is linked to a second dimerization domain. The term “dimerization domain” as used herein refers to the peptide domain which is capable of associating with each other to form a dimer, or in some examples, enables spontaneous dimerization of two peptides.

In certain embodiments, the first dimerization domain can be associated with the second dimerization domain. The association can be via any suitable interaction or linkage or bonding, for example, via a connecter, a disulphide bond, a hydrogen bond, electrostatic interaction, a salt bridge, or hydrophobic-hydrophilic interaction, or the combination thereof. Exemplary dimerization domains include, without limitation, antibody hinge region, an antibody CH2 domain, an antibody CH3 domain, and other suitable protein monomers capable of dimerizing and associating with each other. Hinge region, CH2 and/or CH3 domain can be derived from any antibody isotypes, such as IgG1, IgG2, and IgG4.

A “disulphide bond” refers to a covalent bond with the structure R-S-S-R′. The amino acid cysteine comprises a thiol group that can form a disulphide bond with a second thiol group, for example from another cysteine residue. The disulphide bond can be formed between the thiol groups of two cysteine residues residing respectively on the two polypeptide chains, thereby forming an interchain bridge or interchain bond.

A hydrogen bond is formed by electrostatic attraction between two polar groups when a hydrogen atom covalently bound to a highly electronegative atom such as nitrogen, oxygen, or fluorine. A hydrogen bond can be formed in a polypeptide between the backbone oxygens (e.g. chalcogen groups) and amide hydrogens (nitrogen group) of two residues, respectively, such as a nitrogen group in Asn and an oxygen group in His, or an oxygen group in Asn and a nitrogen group in Lys. A hydrogen bond is stronger than a Van der Waals interaction, but weaker than covalent or ionic bonds, and is critical in maintaining the secondary structure and tertiary structure. For example, an alpha helix is formed when the spacing of amino acid residues occurs regularly between positions i and i+4, and a beta sheet is a stretch of peptide chain 3-10 amino acids long formed when two peptides joined by at least two or three backbone hydrogen bonds, forming a twisted, pleated sheet.

Electrostatic interaction is non-covalent interaction and is important in protein folding, stability, flexibility and function, including ionic interactions, hydrogen bonding and halogen bonding. Electrostatic interactions can be formed in a polypeptide, for example, between Lys and Asp, between Lys and Glu, between Glu and Arg, or between Glu, Trp on the first chain and Arg, Val or Thr on the second chain.

A salt bridge is close-range electrostatic interactions that mainly arises from the anionic carboxylate of either Asp or Glu and the cationic ammonium from Lys or the guanidinium of Arg, which are spatially proximal pairs of oppositely charged residues in native protein structures. Charged and polar residues in largely hydrophobic interfaces may act as hot spots for binding. Among others, residues with ionizable side chains such as His, Tyr, and Ser can also participate the formation of a salt bridge.

A hydrophobic interaction can be formed between one or more Val, Tyr and Ala on the first chain and one or more Val, Leu, and Trp on the second chain, or His and Ala on the first chain and Thr and Phe on the second chain (see Brinkmann, et al, 2017, Supra).

In certain embodiments, the first and/or the second dimerization domain comprises at least a portion of an antibody hinge region. In certain embodiments, the first and/or the second dimerization domain may further comprise an antibody CH2 domain, and/or an antibody CH3 domain. In certain embodiments, the first and/or the second dimerization domain comprises at least a portion of Hinge-Fc region, i.e. Hinge-CH2-CH3 domain. In certain embodiments, the first dimerization domain can be operably linked to the C terminal of the first TCR constant region. In certain embodiments, the second dimerization domain can be operably linked to the C terminal of the antibody CH1 constant region of the second antigen-binding moiety.

In the polypeptide complex provided herein, the first dimerization domain is operably linked to the C-terminal of an engineered TCR constant region, and together forms a chimeric constant region. In other words, the chimeric constant region comprises the first dimerization domain operably linked with the engineered TCR constant region.

In certain embodiments, the chimeric constant region comprises an engineered CBeta attached to the first hinge-Fc region derived from IgG1, IgG2 or IgG4.

In certain embodiments, the chimeric constant region further comprises a first antibody CH2 domain, and/or a first antibody CH3 domain. For example, the chimeric constant region further comprises a first antibody CH2-CH3 domain attached to the C-terminus of the third conjunction domain.

These pairs of chimeric constant regions and second TCR constant domains are useful in that they can be manipulated to fuse to a desired antibody variable region, so as to provide for the polypeptide complex as disclosed herein. For example, an antibody heavy chain variable region can be fused to the chimeric constant region (comprising C1), thereby rendering the first polypeptide chain of the polypeptide complex provided herein; and similarly, an antibody light chain variable region can be fused to the second TCR constant domain (comprising C2), thereby rendering the second polypeptide chain of the polypeptide complex provided herein.

In certain embodiments, the second dimerization domain comprises a hinge region. The hinge region may derived from an antibody, such as IgG1, IgG2, or IgG4. In certain embodiments, the second dimerization domain may optionally further comprise an antibody CH2 domain, and/or an antibody CH3 domain, for example such as a hinge-Fc region. The hinge region may be attached to the antibody heavy chain of the second antigen binding site (e.g. Fab).

In the bispecific polypeptide complex, the first and the second dimerization domain are capable of associating into a dimer. In certain embodiments, the first and the second dimerization domains are different and associate in a way that discourages homodimerization and/or favors heterodimerization. For example, the first and the second dimerization domains can be selected so that they are not identical and that they preferentially form heterodimers between each other rather than to form homodimers within themselves. In certain embodiments, the first and the second dimerization domains are capable of associating into heterodimers via formation of knob-into-hole, hydrophobic interaction, electrostatic interaction, hydrophilic interaction, or increased flexibility.

In certain embodiments, the first and the second dimerization domains comprise CH2 and/or CH3 domains which are respectively mutated to be capable of forming a knobs-into-holes. A knob can be obtained by replacement of a small amino acid residue with a larger one in the first CH2/CH3 polypeptide, and a hole can be obtained by replacement of a large residue with a smaller one. For details of the mutation sites for knobs into holes please see Ridgway et al., 1996, supra, Spiess et al., 2015, supra and Brinkmann et al., 2017, supra.

Bispecific Format

In the polypeptide complex provided herein, the first antigen-binding moiety and the second binding moiety are associated into an Ig-like structure. An Ig-like structure is like a natural antibody having a Y shaped construct, with two arms for antigen-binding and one stem for association and stabilization. The resemblance to natural antibody can provide for various advantages such as good in vivo pharmakinetics, desired immunological response and stability etc. It has been found that the Ig-like structure comprising the first antigen-binding moiety provided herein associated with the second antigen-binding moiety provided herein has thermal stability which is comparable to that of an Ig (e.g. an IgG). In certain embodiments, the Ig-like structure provided herein is at least 70%, 80%, 90%, 95% or 100% of that of a natural IgG.

The bispecific polypeptide complex provided herein comprises four polypeptide chains: i) VH1-C1-Hinge-CH2-CH3; ii) VL1-C2; iii) VH2-CH1-Hinge-CH2-CH3, and iv) VL2-CL, wherein the C1 and C2 are capable of forming a dimer comprising at least one non-native interchain bond, and the two hinge regions and/or the two CH3 domains are capable of forming one or more interchain bond that can facilitate dimerization.

The bispecific polypeptide complexes disclosed herein have longer in vivo half-life and are relatively easier to manufacture when compared to bispecific polypeptide complexes in other formats.

Bispecific Complex Sequences

In some embodiments, the first antigen-binding moiety of the bispecific complex is capable of specifically binding to CD3, and the second antigen-binding moiety is capable of specifically binding to CD20. In other embodiments, the first antigen-binding moiety of the bispecific complex is capable of specifically binding to CD20, and the second antigen-binding moiety is capable of specifically binding to CD3.

In certain embodiments, the bispecific polypeptide complex comprises a combination of four polypeptide sequences: SEQ ID NO: 81, SEQ ID NO: 82, SEQ ID NO: 83, and SEQ ID NO: 84 (the W3278-T2U3.E17R-1.uIgG4.SP antibody), as shown in Example 2. In certain embodiments, the bispecific polypeptide complex comprises a combination of four polypeptide sequences: SEQ ID NO: 85, SEQ ID NO: 86, SEQ ID NO: 87, and SEQ ID NO: 88 (the W3278-T3U2.F16-1.uIgG4.SP antibody), as shown in Example 2. In certain embodiments, the bispecific polypeptide complex comprises a combination of four polypeptide sequences: SEQ ID NO: 89, SEQ ID NO: 90, SEQ ID NO: 91, and SEQ ID NO: 92, as shown in Example 2. In a certain embodiment, the bispecific polypeptide complex comprises five polypeptide chains: a) the first polypeptide chain with a sequence as set forth in SEQ ID NO: 89; b) the second polypeptide chain with a sequence as set forth in SEQ ID NO: 90; c) the third polypeptide chain with a sequence as set forth in SEQ ID NO: 91; d) the fourth polypeptide chain with a sequence as set forth in SEQ ID NO: 91; and e) the firth polypeptide chain with a sequence as set forth in SEQ ID NO: 92. For instance, in a specific embodiment, the bispecific polypeptide complex is the W3278-U2T3.F18R-1.uIgG4.SP antibody, comprising two anti-CD20 binding moieties, the heavy chain VH-CH1 domain of one of which is operably linked to the heavy chain VH domain of the anti-CD3 binding moiety, as shown in Example 2. In certain embodiments, the bispecific polypeptide complex comprises a combination of four polypeptide sequences: SEQ ID NO: 93, SEQ ID NO: 94, SEQ ID NO: 95, and SEQ ID NO: 96 (the W3278-U3T2.F18R-1.uIgG4.SP antibody), as shown in Example 2. In certain embodiments, the bispecific polypeptide complex comprises a combination of four polypeptide sequences: SEQ ID NO: 97, SEQ ID NO: 98, SEQ ID NO: 99, and SEQ ID NO: 100 (the W3278-T3U2.F17R-1.uIgG4.SP antibody), as shown in Example 2. In such embodiments, the first antigen binding moiety binds to CD3, and the second antigen binding moiety binds to CD20.

In certain embodiments, the bispecific polypeptide complex comprises four polypeptide chains comprising: i) VH1 operably linked to a first chimeric constant region; ii) VL1 operably linked to a second chimeric constant region; iii) VH2 operably linked to conventional antibody heavy chain constant region, and iv) VL2 operably linked to conventional antibody light chain constant region. In certain embodiments, the first chimeric constant region can comprise C1-Hinge-CH2-CH3, each as defined supra. In certain embodiments, the second chimeric constant region can comprise C2, as defined supra. In certain embodiments, the conventional antibody heavy chain constant region can comprise CH1-Hinge-CH2-CH3, each as defined supra. In certain embodiments, the conventional antibody light chain constant region can comprise CL, as defined supra.

In certain embodiments, one or more amino acids from the natural glycosylation site at positions 182, 193, 203, 206 and 207 in the polypeptide sequence of SEQ ID NO: 92 are modified. Preferably, the modification is made to the amino acid at position 193 of SEQ ID NO: 92. In certain embodiments, such modification includes one or more mutations of S182X, S193X, S203X, S206X or S207X, wherein X represents any amino acid other than Ser and Thr. In certain preferred embodiments, said modification is S193X, wherein X is selected from Ala, Gly, Pro or Val. In certain embodiments, the above mutation(s) remove an O-glycosylation site, and the type of O-glycosylation is O-saccharide in a Corel configuration and has a structural formula of NeuAc-Gal-GalNAc or NeuAc-Gal-(NeuAc) GalNAc.

As described above, as compared with the bispecific antibodies without modification, the mutant bispecific antibody which is produced by modifying the natural glycosylation site in a corresponding polypeptide sequence of the bispecific antibodies described herein is more similar to natural antibodies, has significantly reduced immunogenicity, improved half-life and improved druggability.

Method of Preparation

The present disclosure provides isolated nucleic acids or polynucleotides that encode the polypeptide complex, and the bispecific anti-CD3×CD20 polypeptide complex provided herein.

The term “nucleic acid” or “polynucleotide” as used herein refers to deoxyribonucleic acids (DNA) or ribonucleic acids (RNA) and polymers thereof in either single- or double-stranded form. Unless specifically limited, the term encompasses polynucleotides containing known analogues of natural nucleotides that have similar binding properties as the reference nucleic acid and are metabolized in a manner similar to naturally occurring nucleotides. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The nucleic acids or polynucleotides encoding the polypeptide complex and the bispecific polypeptide complex provided herein can be constructed using recombinant techniques. To this end, DNA encoding an antigen-binding moiety of a parent antibody (such as CDR or variable region) can be isolated and sequenced using conventional procedures (e.g., by using oligonucleotide probes that are capable of binding specifically to genes encoding the heavy and light chains of the antibody). Likewise, DNA encoding a TCR constant region can also be obtained. As an example, the polynucleotide sequence encoding the variable domain (VH) and the polynucleotide sequence encoding the first TCR constant region (C1) are obtained and operably linked to allow transcription and expression in a host cell to produce the first polypeptide. Similarly, polynucleotide sequence encoding VL are operably linked to polynucleotide sequence encoding C1, so as to allow expression of the second polypeptide in the host cell. If needed, encoding polynucleotide sequences for one or more spacers are also operably linked to the other encoding sequences to allow expression of the desired product.

The encoding polynucleotide sequences can be further operably linked to one or more regulatory sequences, optionally in an expression vector, such that the expression or production of the first and the second polypeptides is feasible and under proper control.

The encoding polynucleotide sequence(s) can be inserted into a vector for further cloning (amplification of the DNA) or for expression, using recombinant techniques known in the art. In another embodiment, the polypeptide complex and the bispecific polypeptide complex provided herein may be produced by homologous recombination known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The term “vector” as used herein refers to a vehicle into which a polynucleotide encoding a protein may be operably inserted so as to bring about the expression of that protein. Typically, the construct also includes appropriate regulatory sequences. For example, the polynucleotide molecule can include regulatory sequences located in the 5′-flanking region of the nucleotide sequence encoding the guide RNA and/or the nucleotide sequence encoding a site-directed modifying polypeptide, operably linked to the coding sequences in a manner capable of expressing the desired transcript/gene in a host cell. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovaviruses (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

In some embodiments, the vector system includes mammalian, bacterial, yeast systems, etc., and comprises plasmids such as, but not limited to, pALTER, pBAD, pcDNA, pCal, pL, pET, pGEMEX, pGEX, pCI, pCMV, pEGFP, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS420, pLexA, pACT2.2 etc., and other laboratorial and commercially available vectors. Suitable vectors may include, plasmid, or viral vectors (e.g., replication defective retroviruses, adenoviruses and adeno-associated viruses).

Vectors comprising the polynucleotide sequence(s) provided herein can be introduced to a host cell for cloning or gene expression. The phrase “host cell” as used herein refers to a cell into which an exogenous polynucleotide and/or a vector has been introduced.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for the vectors encoding the polypeptide complex and the bispecific polypeptide complex. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated polypeptide complex and the bispecific polypeptide complex provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)), such as Expi293; baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors can be cultured in conventional nutrient media modified as appropriate for inducing promoters, selecting transformants, or amplifying the cloning vectors.

For production of the polypeptide complex and the bispecific polypeptide complex provided herein, the host cells transformed with the expression vector may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In one aspect, the present disclosure provides a method of expressing the polypeptide complex and the bispecific polypeptide complex provided herein, comprising culturing the host cell provided herein under the condition at which the polypeptide complex, or the bispecific polypeptide complex is expressed.

In certain embodiments, the present disclosure provides a method of producing the bispecific polypeptide complex provided herein, comprising a) introducing to a host cell: one or more polynucleotides encoding a first antigen-binding moiety comprising a first polynucleotide encoding a first polypeptide comprising, from N-terminus to C-terminus, a first heavy chain variable domain (VH) of a first antibody operably linked to a first TCR constant region (C1), a second polynucleotide encoding a second polypeptide comprising, from N-terminus to C-terminus, a first light chain variable domain (VL) of the first antibody operably linked to a second TCR constant region (C2), and one or more additional polynucleotides encoding a second antigen-binding moiety, wherein C1 and C2 are capable of forming a dimer, and the non-native interchain disulphide bond is capable of stabilizing the dimer of C1 and C2, the first antigen-binding moiety and the second antigen-binding moiety have reduced mispairing than otherwise would have been if both the first antigen-binding moiety and the second antigen-binding moieties were a natural Fab counterparts, and the first antibody has a first antigenic specificity and the second antibody has a second antigenic specificity, b) allowing the host cell to express the bispecific polypeptide complex.

In certain embodiments, the method further comprises isolating the bispecific polypeptide complex.

When using recombinant techniques, the bispecific polypeptide complex provided herein can be produced intracellularly, in the periplasmic space, or directly secreted into the medium. If the product is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the product is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The bispecific polypeptide complex provided herein prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

Where the bispecific polypeptide complex provided herein comprises immunoglobulin Fc domain, then protein A can be used as an affinity ligand, depending on the species and isotype of the Fc domain that is present in the polypeptide complex. Protein A can be used for purification of polypeptide complexes based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose.

Where the bispecific polypeptide complex provided herein comprises a CH3 domain, the Bakerbond ABX resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE′ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the polypeptide complex of interest and contaminants may be subjected to low pH hydrophobic interaction chromatography using an elution buffer at a pH between about 2.5-4.5, preferably performed at low salt concentrations (e.g., from about 0-0.25M salt).

In certain embodiments, the bispecific polypeptide complex provided herein can be readily purified with high yields using conventional methods. One of the advantages of the bispecific polypeptide complex is the significantly reduced mispairing between heavy chain and light chain variable domain sequences. This reduces production of unwanted byproducts and make it possible to obtain high purity product in high yields using relatively simple purification processes.

Derivatives

In certain embodiments, the bispecific polypeptide complex can be used as the base of conjugation with desired conjugates.

It is contemplated that a variety of conjugates may be linked to the polypeptide complex or the bispecific polypeptide complex provided herein (see, for example, “Conjugate Vaccines”, Contributions to Microbiology and Immunology, J. M. Cruse and R. E. Lewis, Jr. (eds.), Carger Press, New York, (1989)). These conjugates may be linked to the polypeptide complex or the bispecific polypeptide complex by covalent binding, affinity binding, intercalation, coordinate binding, complexation, association, blending, or addition, among other methods.

In certain embodiments, the bispecific polypeptide complex provided herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate.

In certain embodiments, the bispecific polypeptide complex may be linked to a conjugate directly, or indirectly for example through another conjugate or through a linker.

For example, the bispecific polypeptide complex having a reactive residue such as cysteine may be linked to a thiol-reactive agent in which the reactive group is, for example, a maleimide, an iodoacetamide, a pyridyl disulphide, or other thiol-reactive conjugation partner (Haugland, 2003, Molecular Probes Handbook of Fluorescent Probes and Research Chemicals, Molecular Probes, Inc.; Brinkley, 1992, Bioconjugate Chem. 3:2; Garman, 1997, Non-Radioactive Labelling: A Practical Approach, Academic Press, London; Means (1990) Bioconjugate Chem. 1:2; Hermanson, G. in Bioconjugate Techniques (1996) Academic Press, San Diego, pp. 40-55, 643-671).

For another example, the bispecific polypeptide complex may be conjugated to biotin, then indirectly conjugated to a second conjugate that is conjugated to avidin. For still another example, the polypeptide complex or the bispecific polypeptide complex may be linked to a linker which further links to the conjugate. Examples of linkers include bifunctional coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suherate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and his-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 (1978)) and N-succinimidyl-4-(2-pyridylthio)pentanoate (SPP) to provide for a disulphide linkage.

The conjugate can be a detectable label, a pharmacokinetic modifying moiety, a purification moiety, or a cytotoxic moiety. Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or O-D-galactosidase), radioisotopes (e.g. ¹²³I, ¹²⁴I, ¹²⁵I, ¹³¹I, ³⁵S, ³H ¹¹¹In, ¹¹²In, ¹⁴C, ⁶⁴Cn, ⁶⁷Cu, ⁸⁶Y, ⁸⁸Y, ⁹⁰Y, ¹⁷⁷Lu, ²¹¹At, ¹⁸⁶Re, ¹⁸⁸Re ¹⁵³Sm, ²¹²Bi, and ³²P, other lanthanides, luminescent labels), chromophoric moiety, digoxigenin, biotin/avidin, a DNA molecule or gold for detection. In certain embodiments, the conjugate can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like. In certain embodiments, the conjugate can be a purification moiety such as a magnetic bead. A “cytotoxic moiety” can be any agent that is detrimental to cells or that can damage or kill cells. Examples of cytotoxic moiety include, without limitation, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Methods for the conjugation of conjugates to proteins such as antibodies, immunoglobulins or fragments thereof are found, for example, in U.S. Pat. Nos. 5,208,020; 6,441,163; WO2005037992; WO2005081711; and WO2006/034488, which are incorporated herein by reference to the entirety.

Pharmaceutical Composition

The present disclosure also provides a pharmaceutical composition comprising the bispecific polypeptide complex provided herein and a pharmaceutically acceptable carrier.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

A “pharmaceutically acceptable carrier” refers to an ingredient in a pharmaceutical formulation, other than an active ingredient, which is bioactivity acceptable and nontoxic to a subject. Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a pharmaceutical composition provided herein decreases oxidation of the polypeptide complex or the bispecific polypeptide complex. This reduction in oxidation prevents or reduces loss of binding affinity, thereby improving protein stability and maximizing shelf-life. Therefore, in certain embodiments, compositions are provided that comprise the polypeptide complex or the bispecific polypeptide complex disclosed herein and one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving the polypeptide complex or the bispecific polypeptide complex as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the polypeptide complex, the bispecific polypeptide complex provided herein or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

Method of Treatment

Therapeutic methods are also provided, comprising: administering a therapeutically effective amount of the polypeptide complex or the bispecific polypeptide complex provided herein to a subject in need thereof, thereby treating or preventing a condition or a disorder. In certain embodiments, the subject has been identified as having a disorder or condition likely to respond to the polypeptide complex or the bispecific polypeptide complex provided herein.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The therapeutically effective amount of the bispecific polypeptide complex provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the bispecific polypeptide complex provided herein may be administered at a therapeutically effective dosage of about 0.01 mg/kg to about 100 mg/kg (e.g., about 0.01 mg/kg, about 0.5 mg/kg, about 1 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg). In certain of these embodiments, the polypeptide complex or the bispecific polypeptide complex provided herein is administered at a dosage of about 50 mg/kg or less, and in certain of these embodiments the dosage is 10 mg/kg or less, 5 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The bispecific polypeptide complex provided herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In certain embodiments, the condition or disorder treated by the bispecific polypeptide complex provided herein is cancer or a cancerous condition, autoimmune diseases, infectious and parasitic diseases, cardiovascular diseases, neuropathies, neuropsychiatric conditions, injuries, inflammations, or coagulation disorder.

“Cancer” or “cancerous condition” as used herein refers to any medical condition mediated by neoplastic or malignant cell growth, proliferation, or metastasis, and includes both solid cancers and non-solid cancers such as leukemia. “Tumor” as used herein refers to a solid mass of neoplastic and/or malignant cells.

With regard to cancer, “treating” or “treatment” may refer to inhibiting or slowing neoplastic or malignant cell growth, proliferation, or metastasis, preventing or delaying the development of neoplastic or malignant cell growth, proliferation, or metastasis, or some combination thereof. With regard to a tumor, “treating” or “treatment” includes eradicating all or part of a tumor, inhibiting or slowing tumor growth and metastasis, preventing or delaying the development of a tumor, or some combination thereof.

For example, with regard to the use of the bispecific polypeptide complex disclosed herein to treat cancer, a therapeutically effective amount is the dosage or concentration of the polypeptide complex capable of eradicating all or part of a tumor, inhibiting or slowing tumor growth, inhibiting growth or proliferation of cells mediating a cancerous condition, inhibiting tumor cell metastasis, ameliorating any symptom or marker associated with a tumor or cancerous condition, preventing or delaying the development of a tumor or cancerous condition, or some combination thereof.

In certain embodiments, the conditions and disorders include tumors and cancers, for example, non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, brain stem glioma.

In certain embodiments, the conditions and disorders include CD20-related condition, such as, B cell lymphoma, optionally Hodgkin lymphoma or non-Hodgkin lymphoma, wherein the non-Hodgkin lymphoma comprises: Diffuse large B-cell lymphoma (DLBCL), Follicular lymphoma, Marginal zone B-cell lymphoma (MZL), Mucosa-Associated Lymphatic Tissue lymphoma (MALT), Small lymphocytic lymphoma (chronic lymphocytic leukemia, CLL), or Mantle cell lymphoma (MCL), Acute Lymphoblastic Leukemia (ALL), or Waldenstrom's Macroglobulinemia (WM).

The bispecific polypeptide complex may be administered alone or in combination with one or more additional therapeutic means or agents.

In certain embodiments, when used for treating cancer or tumor or prolierative disease, the bispecific polypeptide complex provided herein may be administered in combination with chemotherapy, radiation therapy, surgery for the treatment of cancer (e.g., tumorectomy), one or more anti-emetics or other treatments for complications arising from chemotherapy, or any other therapeutic agent for use in the treatment of cancer or any medical disorder that related. “Administered in combination” as used herein includes administration simultaneously as part of the same pharmaceutical composition, simultaneously as separate compositions, or at different timings as separate compositions. A composition administered prior to or after another agent is considered to be administered “in combination” with that agent as the phrase is used herein, even if the composition and the second agent are administered via different routes. Where possible, additional therapeutic agents administered in combination with the polypeptide complex or the bispecific polypeptide complex provided herein are administered according to the schedule listed in the product information sheet of the additional therapeutic agent, or according to the Physicians' Desk Reference (Physicians' Desk Reference, 70th Ed (2016)) or protocols well known in the art.

In certain embodiments, the therapeutic agents can induce or boost immune response against cancer. For example, a tumor vaccine can be used to induce immune response to certain tumor or cancer. Cytokine therapy can also be used to enhance tumor antigen presentation to the immune system. Examples of cytokine therapy include, without limitation, interferons such as interferon-α, -β, and -γ, colony stimulating factors such as macrophage-CSF, granulocyte macrophage CSF, and granulocyte-CSF, interleukins such IL-1, IL-la, IL-2, IL-3, IL-4, IL-5, IL-6, IL-7, IL-8, IL-9, IL-10, IL-11, and IL-12, tumor necrosis factors such as TNF-α and TNF-β. Agents that inactivate immunosuppressive targets can also be used, for example, TGF-beta inhibitors, IL-10 inhibitors, and Fas ligand inhibitors. Another group of agents include those that activate immune responsiveness to tumor or cancer cells, for example, those enhance T cell activation (e.g. agonist of T cell costimulatory molecules such as CTLA-4, ICOS and OX-40), and those enhance dendritic cell function and antigen presentation.

Kits

The present disclosure further provides kits comprising the bispecific polypeptide complex provided herein. In some embodiments, the kits are useful for detecting the presence or level of, or capturing or enriching one or more target of interest in a biological sample. The biological sample can comprise a cell or a tissue.

In some embodiments, the kit comprises the bispecific polypeptide complex provided herein which is conjugated with a detectable label. In certain other embodiments, the kit comprises an unlabeled bispecific polypeptide complex provided herein, and further comprises a secondary labeled antibody which is capable of binding to the unlabeled bispecific polypeptide complex provided herein. The kit may further comprise an instruction of use, and a package that separates each of the components in the kit.

In certain embodiments, the bispecific polypeptide complex provided herein are associated with a substrate or a device. Useful substrate or device can be, for example, magnetic beads, microtiter plate, or test strip. Such can be useful for a binding assay (such as ELISA), an immunographic assay, capturing or enriching of a target molecule in a biological sample.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Preparation of Materials and Benchmark Antibodies

### 1. Preparation of Materials

Information on the commercially available materials used in the examples is provided in Table 1.

TABLE 1
The commercial materials

| Materials | Vendor | Cat. |
|---|---|---|
| CD4⁺ T Cell Isolation Kit (Human) | Stemcell | 19052 |
| CD8⁺ T cell Isolation Kit (Human) | Stemcell | 19053 |
| Calcein-AM | Invitrogen | C3099 |
| CellTracker ™ FarRed | Invitrogen | C34572 |
| Propidium Iodide (PI) | Invitrogen | P3566 |
| Alexa Fluor647 conjugated Goat | Jackson | 109-605-098 |
| anti-human IgG Fc | | |
| FITC labeled anti-human CD4 | BD Pharmingen | 550628 |
| PerCP-Cy5.5 labeled anti-human | BD Pharmingen | 565310 |
| CD8 | | |
| PE labeled anti-human CD69 | BD Pharmingen | 555531 |
| APC labeled anti-human CD25 | BD Pharmingen | 555434 |
| Capture Antibody Purified Anti- | BD Pharmingen | 555212 |
| Human TNF | | (51-26371E) |
| Detection Antibody Biotinylated | BD Pharmingen | 555212 |
| Anti-Human TNF monoclonal | | (51-26372E) |
| antibody | | |
| Enzyme Reagent Streptavidin-HRP | BD Pharmingen | 555212 |
| | | (51-9002813) |
| Recombinant Human TNF Standard | BD Pharmingen | 555212 |
| | | (51-26376E) |
| Human/Primate IL-2 Antibody mAb | R&D | MAB602 |
| Mouse IgG2A | | |
| Human/Primate IL-2 Biotinylated | R&D | BAF202 |
| Antibody | | |
| Recombinant human IL-2 | R&D | 202-IL |
| Jurkat | ATCC | TIB-152 |
| Raji | ATCC | CCL-86 |
| NAMALWA | ATCC | CRL-1432 |
| Ramos | ATCC | CRL-1596 |
| SU-DHL-1 | ATCC | CRL-2955 |

### 2. Generation of Benchmark Antibodies

Two benchmark antibodies W327-BMK1 and W327-BMK4 were applied in the examples as reference antibodies.

Anti-human CD20 benchmark antibody BMK1 (Rituximab) was generated based on the sequences of clone C2B8 from US Patent Application US 20140004037 A1. Anti-CD3×CD20 reference bispecific antibody BMK4 (REGN1979) genes were synthesized according to the sequences in US Patent Application US 20150266966 A1. The BMK antibodies were expressed from Expi293 cells and then purified using Protein A chromatography

### Example 2

### Preparation of Bispecific Antibodies of the Present Disclosure

### 1. Design and Engineering of Antibody and TCR Chimeric Proteins

TCR Sequences

TCRs are heterodimeric proteins made up of two chains. About 95% human T cells have TCRs consisting of alpha and beta chains. Considering that more crystal structures are available for beta chain TRBC1, TRBC1 sequences were chosen as the major backbone to design the polypeptide complex disclosed herein (“WuXiBody”). A typical amino acid sequence of TRBC1 can be found in Protein Data Bgank (PDB) structure 4L4T.

Interchain Disulphide-Bond of TCR

TCR crystal structures were used to guide our WuXiBody design. Unlike native TCR anchored on the membrane of T cell surface, soluble TCR molecules are less stable, although its 3D structure is very similar to antibody Fab. As a matter of fact, the instability of TCR in soluble condition used to be a big obstacle that prevents the elucidation of its crystal structure (see Wang, Protein Cell, 5(9), pp. 649-652 (2014)). We adopted a strategy of introducing a pair of Cys mutations in the TCR constant region and found it can significantly improve chain assembly and enhance expression.

The conjunctions connecting antibody variable and TCR constant domains, their relative fusion orientations, as well as the Fc-connecting conjunctions were all carefully fine-turned. As TCR structure is very similar to antibody Fab, we superimposed the antibody Fv homology model on TCR variable region (PDB 4L4T). The superimposed structure indicates that antibody Fv is structurally compatible with TCR constant domain. Based on this structural alignment and corresponding sequences, all the relevant engineering parameters were designed.

2. Preparation of Bispecific Antibodies of the Present Disclosure

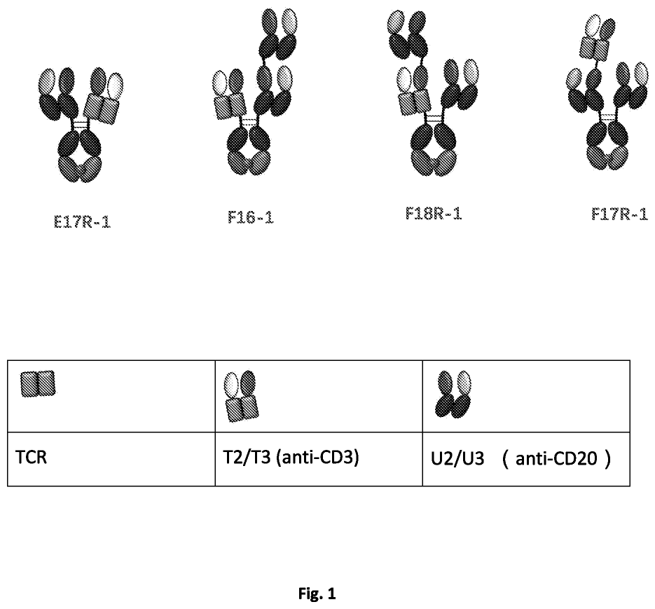

The W3278 BsAbs were produced as human IgG4 in a knobs-into-holes format (S. Atwell, J. B. Ridgway, J. A. Wells, P. Carter, Stable heterodimers from remodeling the domain interface of a homodimer using a phage display library. J. Mol. Biol. 270, 26-35 (1997); C. Spiess, M. Merchant, A. Huang, et al. D. G. Yansura, J. M. Scheer, Bispecific antibodies with natural architecture produced by co-culture of bacteria expressing two distinct half-antibodies. Nat. Biotechnol. 31, 753-758 (2013)). The human IgG4 Fc region sequence was designed with a S228P mutation. The anti-CD3 monoclonal antibody was generated through immunization of mice with human CD3ε and CD3δ ECD proteins by hybridoma technology from an in house program. The anti-CD20 arm variable region sequences were based on the sequences of Ofatumumab (clone 2F2 from PCT Publication No. WO 2010083365A1) or Rituximab (clone C2B8 from US Patent Application US 20140004037 A1). The sequences of W3278 BsAb candidates were listed in Table 2, the DNA sequences of which were synthesized at Genewiz (Shanghai) and cloned into modified pcDNA3.3 expression vector. The expression vectors of anti-CD20 arm and anti-CD3 arm were co-transfected into Expi293 (Invitrogen-A14527) by using ExpiFectamine293 Transfection Kit (Invitrogen-A14524). The cells were cultured in Expi293 Expression Medium (Invitrogen-A1435101) on an orbital shaker platform rotating at 135 rpm in a 37° C. incubator containing a humidified atmosphere with 8% CO₂. The culture supernatants were harvested for protein purification using Protein A column (GE Healthcare, 17543802). The protein concentration was measured by UV-Vis spectrophotometer (NanoDrop 2000, Thermo Scientific). The protein purity was estimated by SDS-PAGE and analytic HPLC-SEC. The schematic diagrams of W3278-BsAb candidates are described in FIG. 1.

TABLE 2
The Sequences of heavy and/or light chains in each BsAb

| | | SEQ | |
|---|---|---|---|
| | | ID | |
| Clone ID | | NO | Amino acid sequence |
| W3278- | H1 | 81 | QVQLVQSGAEVKKPGSSVKVSCKASGYSFTTYYIHWVRQAP |
| T2U3.E17R- | | | GQGLEWMGWIFPGNDNIKYSEKFKGRVTITADKSTSTAYME |
| 1.uIgG4.SP | | | LSSLRSEDTAVYYCAIDSVSIYYFDYWGQGTLVTVSSASTKG |
| | | | PSVFPLAPCSRSTSESTAALGCLVKDYFPEPVTVSWNSGALTS |
| | | | GVHTFPAVLQSSGLYSLSSVVTVPSSSLGTKTYTCNVDHKPS |
| | | | NTKVDKRVESKYGPPCPPCPAPEFLGGPSVFLFPPKPKDTLMI |
| | | | SRTPEVTCVVVDVSQEDPEVQFNWYVDGVEVHNAKTKPRE |
| | | | EQFNSTYRVVSVLTVLHQDWLNGKEYKCKVSNKGLPSSIEK |
| | | | TISKAKGQPREPQVYTLPPCQEEMTKNQVSLWCLVKGFYPS |
| | | | DIAVEWESNGQPENNYKTTPPVLDSDGSFFLYSRLTVDKSR |
| | | | WQEGNVFSCSVMHEALHNHYTQKSLSLSLG |
| | H2 | 82 | EVQLVESGGGLVQPGRSLRLSCAASGFTFNDYAMHWVRQA |
| | | | PGKGLEWVSTISWNSGSIGYADSVKGRFTISRDNAKKSLYLQ |
| | | | MNSLRAEDTALYYCAKDIQYGNYYYGMDVWGQGTTVTV [symbol] |
| | | | [symbol] [symbol] [symbol] [symbol] |
| | | | [symbol] [symbol] [symbol] [symbol] |
| | | | [symbol] [symbol] [symbol] [symbol] [symbol] |
| | | | [symbol] WGRYGPPCPPCPAPEFLGGPSVFLEPPKPKDTLMISRTPEV |
| | | | TCVVVDVSQEDPEVQFNWYVDGVEVHNAKTKPREEQFNST |
| | | | YRVVSVLTVLHQDWLNGKEYKCKVSNKGLPSSIEKTISKAK |
| | | | GQPREPQVCTLPPSQEEMTKNQVSLSCAVKGFYPSDIAVEWE |
| | | | SNGQPENNYKTTPPVLDSDGSFFLVSRLTVDKSRWQEGNVFS |
| | | | CSVMHEALHNHYTQKSLSLSLG |
| | L1 | 83 | DIVMTQSPDSLAVSLGERATINCKSSQSLLNSRTRKNYLAWY |
| | | | QQKPGQPPKLLIYWASTRKSGVPDRFSGSGSGTDFTLTISSLQ |
| | | | AEDVAVYYCTQSFILRTFGGGTKVEIKRTVAAPSVFIFPPSDE |
| | | | QLKSGTASVVCLLNNFYPREAKVQWKVDNALQSGNSQESV |
| | | | TEQDSKDSTYSLSSTLTLSKADYEKHKVYACEVTHQGLSSPV |
| | | | TKSFNRGEC |
| | L2 | 84 | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPITFGQGTRLEIK [symbol] [symbol] |
| | | | [symbol] [symbol] [symbol] [symbol] |
| | | | [symbol] [symbol] [symbol] |
| W3278- | H1 | 85 | QVQLVQSGAEVKKPGSSVKVSCKASGFAFTDYYIHWVRQAP |
| T3U2.F16- | | | GQGLEWMGWISPGNVNTKYNENFKGRVTITADKSTSTAYM |
| 1.uIgG4.SP | | | ELSSLRSEDTAVYYCARDGYSLYYFDYWGQGTLVTV [symbol] |
| | | | [symbol] [symbol] [symbol] [symbol] |
| | | | [symbol] [symbol] [symbol] [symbol] |
| | | | [symbol] [symbol] [symbol] [symbol] W |
| | | | GRYGPPCPPCPAPEFLGGPSVFLEPPKPKDTLMISRTPEVTCV |
| | | | VVDVSQEDPEVQFNWYVDGVEVHNAKTKPREEQFNSTYRV |
| | | | VSVLTVLHQDWLNGKEYKCKVSNKGLPSSIEKTISKAKGQP |
| | | | REPQVYTLPPCQEEMTKNQVSLWCLVKGFYPSDIAVEWESN |
| | | | GQPENNYKTTPPVLDSDGSFFLYSRLTVDKSRWQEGNVFSCS |
| | | | VMHEALHNHYTQKSLSLSLG |
| | H2 | 86 | QVQLQQPGAELVKPGASVKMSCKASGYTFTSYNMHWVKQT |
| | | | PGRGLEWIGAIYPGNGDTSYNQKFKGKATLTADKSSSTAYM |
| | | | QLSSLTSEDSAVYYCARSTYYGGDWYFNVWGAGTTVTVSA |
| | | | ASTKGPSVFPLAPCSRSTSESTAALGCLVKDYFPEPVTVSWN |
| | | | SGALTSGVHTFPAVLQSSGLYSLSSVVTVPSSSLGTKTYTCN |
| | | | VDHKPSNTKVDKRVGGGGSGGGGSQVQLQQPGAELVKPGA |
| | | | SVKMSCKASGYTFTSYNMHWVKQTPGRGLEWIGAIYPGNG |
| | | | DTSYNQKFKGKATLTADKSSSTAYMQLSSLTSEDSAVYYCA |
| | | | RSTYYGGDWYFNVWGAGTTVTVSAASTKGPSVFPLAPCSRS |
| | | | TSESTAALGCLVKDYFPEPVTVSWNSGALTSGVHTFPAVLQS |
| | | | SGLYSLSSVVTVPSSSLGTKTYTCNVDHKPSNTKVDKRVESK |
| | | | YGPPCPPCPAPEFLGGPSVFLFPPKPKDTLMISRTPEVTCVVV |
| | | | DVSQEDPEVQFNWYVDGVEVHNAKTKPREEQFNSTYRVVS |
| | | | VLTVLHQDWLNGKEYKCKVSNKGLPSSIEKTISKAKGQPREP |
| | | | QVCTLPPSQEEMTKNQVSLSCAVKGFYPSDIAVEWESNGQPE |
| | | | NNYKTTPPVLDSDGSFFLVSRLTVDKSRWQEGNVFSCSVMH |
| | | | EALHNHYTQKSLSLSLG |
| | L1 | 87 | DIVMTQSPDSLAVSLGERATINCKSSQSLLNSRTRKNYLAWY |
| | | | QQKPGQPPKLLIYWASTRQSGVPDRFSGSGSGTDFTLTISSLQ [symbol] |
| | | | AEDVAVYYCTQSHTLRTFGGGTKVEIK [symbol] [symbol] |
| | | | [symbol] [symbol] [symbol] [symbol] |
| | | | [symbol] [symbol] [symbol] [symbol] |
| | L2 | 88 | QIVLSQSPAILSASPGEKVTMTCRASSSVSYIHWFQQKPGSSP |
| | | | KPWIYATSNLASGVPVRFSGSGSGTSYSLTISRVEAEDAATY |
| | | | YCQQWTSNPPTFGGGTKLEIKRTVAAPSVFIFPPSDEQLKSGT |
| | | | ASVVCLLNNFYPREAKVQWKVDNALQSGNSQESVTEQDSK |
| | | | DSTYSLSSTLTLSKADYEKHKVYACEVTHQGLSSPVTKSFNR |
| | | | GEC |
| W3278- | H1 | 89 | QVQLQQPGAELVKPGASVKMSCKASGYTFTSYNMHWVKQT |
| U2T3.F18R- | | | PGRGLEWIGAIYPGNGDTSYNQKFKGKATLTADKSSSTAYM |
| 1.uIgG4.SP | | | QLSSLTSEDSAVYYCARSTYYGGDWYFNVWGAGTTVTVSA |
| | | | ASTKGPSVFPLAPCSRSTSESTAALGCLVKDYFPEPVTVSWN |
| | | | SGALTSGVHTFPAVLQSSGLYSLSSVVTVPSSSLGTKTYTCN |
| | | | VDHKPSNTKVDKRVGGGGSGGGGSQVQLVQSGAEVKKPGS |
| | | | SVKVSCKASGFAFTDYYIHWVRQAPGQGLEWMGWISPGNV |
| | | | NTKYNENFKGRVTITADKSTSTAYMELSSLRSEDTAVYYCA |
| | | | RDGYSLYYFDYWGQGTLVTV [symbol] [symbol] |
| | | | [symbol] [symbol] [symbol] [symbol] |
| | | | [symbol] [symbol] [symbol] [symbol] |
| | | | [symbol] [symbol] WGRYGPPCPPCPAPEFLGG |
| | | | PSVFLFPPKPKDTLMISRTPEVTCVVVDVSQEDPEVQFNWYV |
| | | | DGVEVHNAKTKPREEQFNSTYRVVSVLTVLHQDWLNGKEY |
| | | | KCKVSNKGLPSSIEKTISKAKGQPREPQVYTLPPCQEEMTKN |
| | | | QVSLWCLVKGFYPSDIAVEWESNGQPENNYKTTPPVLDSDG |
| | | | SFFLYSRLTVDKSRWQEGNVFSCSVMHEALHNHYTQKSLSL |
| | | | SLG |
| | H2 | 90 | QVQLQQPGAELVKPGASVKMSCKASGYTFTSYNMHWVKQT |
| | | | PGRGLEWIGAIYPGNGDTSYNQKFKGKATLTADKSSSTAYM |
| | | | QLSSLTSEDSAVYYCARSTYYGGDWYFNVWGAGTTVTVSA |
| | | | ASTKGPSVFPLAPCSRSTSESTAALGCLVKDYFPEPVTVSWN |
| | | | SGALTSGVHTFPAVLQSSGLYSLSSVVTVPSSSLGTKTYTCN |
| | | | VDHKPSNTKVDKRVESKYGPPCPPCPAPEFLGGPSVFLFPPKP |
| | | | KDTLMISRTPEVTCVVVDVSQEDPEVQFNWYVDGVEVHNA |
| | | | KTKPREEQFNSTYRVVSVLTVLHQDWLNGKEYKCKVSNKG |
| | | | LPSSIEKTISKAKGQPREPQVCTLPPSQEEMTKNQVSLSCAVK |
| | | | GFYPSDIAVEWESNGQPENNYKTTPPVLDSDGSFFLVSRLTV |
| | | | DKSRWQEGNVFSCSVMHEALHNHYTQKSLSLSLG |
| | | | L191QIVLSQSPAILSASPGEKVTMTCRASSSVSYIHWFQQKPGSSP |
| | | | KPWIYATSNLASGVPVRFSGSGSGTSYSLTISRVEAEDAATY |
| | | | YCQQWTSNPPTFGGGTKLEIKRTVAAPSVFIFPPSDEQLKSGT |
| | | | ASVVCLLNNFYPREAKVQWKVDNALQSGNSQESVTEQDSK |
| | | | DSTYSLSSTLTLSKADYEKHKVYACEVTHQGLSSPVTKSFNR |
| | | | GEC |
| | L2 | 92 | DIVMTQSPDSLAVSLGERATINCKSSQSLLNSRTRKNYLAWY |
| | | | QQKPGQPPKLLIYWASTRQSGVPDRFSGSGSGTDFTLTISSLQ |
| | | | AEDVAVYYCTQSHTLRTFGGGTKVEIK [symbol] |
| | | | [symbol] [symbol] [symbol] [symbol] |
| | | | [symbol] [symbol] [symbol] [symbol] |
| W3278- | H1 | 93 | EVQLVESGGGLVQPGRSLRLSCAASGFTFNDYAMHWVRQA |
| U3T2.F18R- | | | PGKGLEWVSTISWNSGSIGYADSVKGRFTISRDNAKKSLYLQ |
| 1.uIgG4.SP | | | MNSLRAEDTALYYCAKDIQYGNYYYGMDVWGQGTTVTVS |
| | | | SASTKGPSVFPLAPCSRSTSESTAALGCLVKDYFPEPVTVSW |
| | | | NSGALTSGVHTFPAVLQSSGLYSLSSVVTVPSSSLGTKTYTC |
| | | | NVDHKPSNTKVDKRVGGGGSGGGGSQVQLVQSGAEVKKPG |
| | | | SSVKVSCKASGYSETTYYIHWVRQAPGQGLEWMGWIFPGN |
| | | | DNIKYSEKFKGRVTITADKSTSTAYMELSSLRSEDTAVYYCA |
| | | | IDSVSIYYFDYWGQGTLVTV [symbol] [symbol] |
| | | | [symbol] [symbol] [symbol] [symbol] |
| | | | [symbol] [symbol] [symbol] [symbol] |
| | | | [symbol] [symbol] WGRYGPPCPPCPAPEFLGG |
| | | | PSVFLEPPKPKDTLMISRTPEVTCVVVDVSQEDPEVQFNWYV |
| | | | DGVEVHNAKTKPREEQFNSTYRVVSVLTVLHQDWLNGKEY |
| | | | KCKVSNKGLPSSIEKTISKAKGQPREPQVYTLPPCQEEMTKN |
| | | | QVSLWCLVKGFYPSDIAVEWESNGQPENNYKTTPPVLDSDG |
| | | | SFFLYSRLTVDKSRWQEGNVFSCSVMHEALHNHYTQKSLSL |
| | | | SLG |
| | H2 | 94 | EVQLVESGGGLVQPGRSLRLSCAASGFTFNDYAMHWVRQA |
| | | | PGKGLEWVSTISWNSGSIGYADSVKGRFTISRDNAKKSLYLQ |
| | | | MNSLRAEDTALYYCAKDIQYGNYYYGMDVWGQGTTVTVS |
| | | | SASTKGPSVFPLAPCSRSTSESTAALGCLVKDYFPEPVTVSW |
| | | | NSGALTSGVHTFPAVLQSSGLYSLSSVVTVPSSSLGTKTYTC |
| | | | NVDHKPSNTKVDKRVESKYGPPCPPCPAPEFLGGPSVFLEPP |
| | | | KPKDTLMISRTPEVTCVVVDVSQEDPEVQFNWYVDGVEVH |
| | | | NAKTKPREEQFNSTYRVVSVLTVLHQDWLNGKEYKCKVSN |
| | | | KGLPSSIEKTISKAKGQPREPQVCTLPPSQEEMTKNQVSLSCA |
| | | | VKGFYPSDIAVEWESNGQPENNYKTTPPVLDSDGSFFLVSRL |
| | | | TVDKSRWQEGNVFSCSVMHEALHNHYTQKSLSLSLG |
| | L1 | 95 | EIVLTQSPATLSLSPGERATLSCRASQSVSSYLAWYQQKPGQ |
| | | | APRLLIYDASNRATGIPARFSGSGSGTDFTLTISSLEPEDFAVY |
| | | | YCQQRSNWPITFGQGTRLEIKRTVAAPSVFIFPPSDEQLKSGT |
| | | | ASVVCLLNNFYPREAKVQWKVDNALQSGNSQESVTEQDSK |
| | | | DSTYSLSSTLTLSKADYEKHKVYACEVTHQGLSSPVTKSFNR |
| | | | GEC |
| | L2 | 96 | DIVMTQSPDSLAVSLGERATINCKSSQSLLNSRTRKNYLAWY |
| | | | QQKPGQPPKLLIYWASTRKSGVPDRFSGSGSGTDFTLTISSLQ |
| | | | AEDVAVYYCTQSFILRTFGGGTKVEIK [symbol] [symbol] |
| | | | [symbol] [symbol] [symbol] [symbol] |
| | | | [symbol] [symbol] [symbol] [symbol] |
| W3278- | H1 | 97 | QVQLVQSGAEVKKPGSSVKVSCKASGFAFTDYYIHWVRQAP |
| T3U2.F17R- | | | GQGLEWMGWISPGNVNTKYNENFKGRVTITADKSTSTAYM |
| 1.uIgG4.SP | | | ELSSLRSEDTAVYYCARDGYSLYYFDYWGQGTLVTV [symbol] |
| | | | [symbol] [symbol] [symbol] [symbol] |
| | | | [symbol] [symbol] [symbol] [symbol] |
| | | | [symbol] [symbol] [symbol] [symbol] |
| | | | GRGGGGSGGGGSQVQLQQPGAELVKPGASVKMSCKASGYT |
| | | | FTSYNMEIWVKQTPGRGLEWIGAIYPGNGDTSYNQKFKGKA |
| | | | TLTADKSSSTAYMQLSSLTSEDSAVYYCARSTYYGGDWYFN |
| | | | VWGAGTTVTVSAASTKGPSVFPLAPCSRSTSESTAALGCLVK |
| | | | DYFPEPVTVSWNSGALTSGVHTFPAVLQSSGLYSLSSVVTVP |
| | | | SSSLGTKTYTCNVDHKPSNTKVDKRVESKYGPPCPPCPAPEF |
| | | | LGGPSVFLEPPKPKDTLMISRTPEVTCVVVDVSQEDPEVQFN |
| | | | WYVDGVEVHNAKTKPREEQFNSTYRVVSVLTVLHQDWLNG |
| | | | KEYKCKVSNKGLPSSIEKTISKAKGQPREPQVYTLPPCQEEM |
| | | | TKNQVSLWCLVKGFYPSDIAVEWESNGQPENNYKTTPPVLD |
| | | | SDGSFFLYSRLTVDKSRWQEGNVFSCSVMHEALHNHYTQKS |
| | | | LSLSLG |
| | H2 | 98 | QVQLQQPGAELVKPGASVKMSCKASGYTFTSYNMHWVKQT |
| | | | PGRGLEWIGAIYPGNGDTSYNQKFKGKATLTADKSSSTAYM |
| | | | QLSSLTSEDSAVYYCARSTYYGGDWYFNVWGAGTTVTVSA |
| | | | ASTKGPSVFPLAPCSRSTSESTAALGCLVKDYFPEPVTVSWN |
| | | | SGALTSGVHTFPAVLQSSGLYSLSSVVTVPSSSLGTKTYTCN |
| | | | VDHKPSNTKVDKRVESKYGPPCPPCPAPEFLGGPSVFLFPPKP |
| | | | KDTLMISRTPEVTCVVVDVSQEDPEVQFNWYVDGVEVHNA |
| | | | KTKPREEQFNSTYRVVSVLTVLHQDWLNGKEYKCKVSNKG |
| | | | LPSSIEKTISKAKGQPREPQVCTLPPSQEEMTKNQVSLSCAVK |
| | | | GFYPSDIAVEWESNGQPENNYKTTPPVLDSDGSFELVSRLTV |
| | | | DKSRWQEGNVFSCSVMHEALHNHYTQKSLSLSLG |
| | L1 | 99 | DIVMTQSPDSLAVSLGERATINCKSSQSLLNSRTRKNYLAWY |
| | | | QQKPGQPPKLLIYWASTRQSGVPDRFSGSGSGTDFTLTISSLQ |
| | | | AEDVAVYYCTQSHTLRTFGGGTKVEIK [symbol] |
| | | | [symbol] [symbol] [symbol] [symbol] |
| | | | [symbol] [symbol] [symbol] [symbol] |
| | L2 | 100 | QIVLSQSPAILSASPGEKVTMTCRASSSVSYIHWFQQKPGSSP |
| | | | KPWIYATSNLASGVPVRFSGSGSGTSYSLTISRVEAEDAATY |
| | | | YCQQWTSNPPTFGGGTKLEIKRTVAAPSVFIFPPSDEQLKSGT |
| | | | ASVVCLLNNFYPREAKVQWKVDNALQSGNSQESVTEQDSK |
| | | | DSTYSLSSTLTLSKADYEKHKVYACEVTHQGLSSPVTKSFNR |
| | | | GEC |

Note:
the TCR sequence sare indicated in Italic script.

Further, for the bispecific antibody, W3278-U2T3.F18R-1.uIgG4.SP, one or more amino acids from the natural glycosylation site at positions 182, 193, 203, 206 and 207 in the polypeptide sequence of SEQ ID NO: 92 were modified. Preferably, the modification was made to the amino acid at position 193 of SEQ ID NO: 92. In certain embodiments, such modification included one or more mutations of S182X, S193X, S203X, S206X or S207X, wherein X represented any amino acid other than Ser and Thr. In certain preferred embodiments, said modification was S193X, wherein X was selected from Ala, Gly, Pro or Val. The above mutation(s) removed an O-glycosylation site, and the type of O-glycosylation was O-saccharide in a Corel configuration and had a structural formula of NeuAc-Gal-GalNAc or NeuAc-Gal-(NeuAc) GalNAc.

As described above, as compared with the bispecific antibodies without modification, the mutant bispecific antibody which was produced by modifying the natural glycosylation site in a corresponding polypeptide sequence of the bispecific antibodies described herein was more similar to natural antibodies, had significantly reduced immunogenicity, improved half-life and improved druggability.

### Example 3

### In Vitro Characterization

### 1. Cell Lines and Primary Cell Isolation

The following cell lines cultured in complete media (RPMI1640 supplemented with 10% FBS, 100 U/ml penicillin, 100 μg/ml streptomycin) were used: Jurkat (CD3+/CD20− cells); Raji, Ramos and NAMALWA (CD20+/CD3− cells), SU-DHL-1 (CD20−/CD3− cells).

Human peripheral blood mononuclear cells (PBMC) were freshly isolated by Ficoll-Paque PLUS (GE Healthcare-17-1440-03) density centrifugation from heparinized venous blood from healthy normal donors. The primary human CD8⁺ T cells were isolated from fresh human PBMC by EasySep kit (Stemcell-19053) and purified CD4⁺ T cells by EasySep (Stemcell-19052) columns.

### 2. Binding of W3278 BsAb to Target Cells

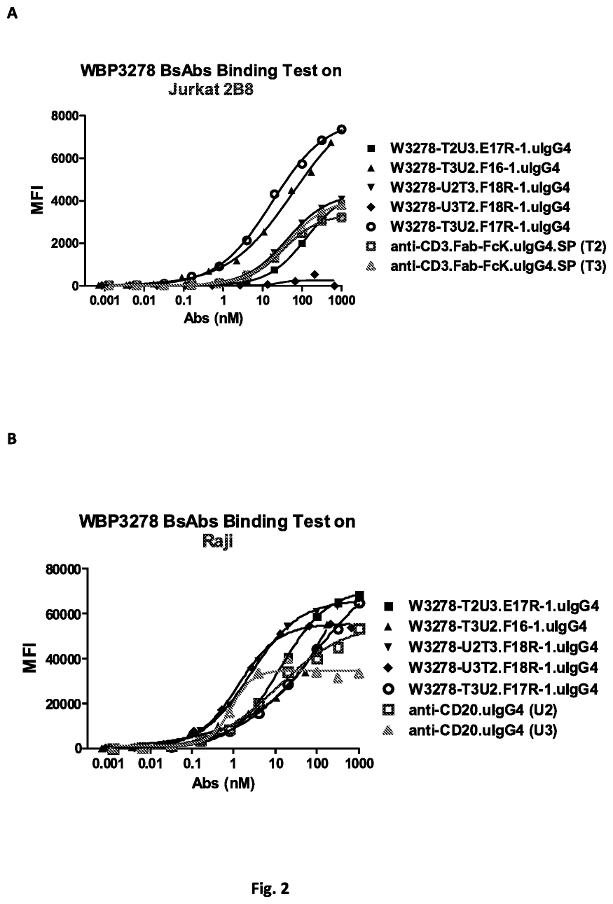

The binding of W3278 BsAb to target cells was determined by flow cytometry. Briefly, 1×10⁵/well of target cells (CD3+/CD20− cells or CD20+/CD3− cells) were incubated with serial dilutions of W3278 BsAb or human IgG4 isotype control antibody at 4° C. for 60 minutes. After incubation, cells were washed twice with cold 1% BSA/1×PBS and Alexa Fluor647 conjugated Goat anti-human IgG Fc (Jackson-109-605-098) was then added and incubated for 30 minutes at 4° C. After washing twice, the geometric mean fluorescence (MFI) of stained cells was measured using a FACS Canto II cytometer (BD Biosciences). Wells containing no antibody or fluorescent secondary antibody only were used to establish background fluorescence. The EC₅₀ values of cell binding were determined using GraphPad Prism 5 software (GraphPad Software, La Jolla, Calif.) with values calculated using a four-parameter non-linear regression analysis. The FACS binding of W3278 BsAb to target cells are shown in FIG. 2 and the binding EC₅₀ shown in Table 3 below.

TABLE 3
The FACS binding EC50 of W3278 BsAbs and the parental
antibodies to cell surface targets.

| | Jurkat 2B8 | | Raji | |
|---|---|---|---|---|
| | EC50 | TOP | EC50 | TOP |
| Abs | (nM) | MFI | (nM) | MFI |
| W3278-T2U3.E17R-1.ulgG4.5P | 128.7 | 3876 | 13.2 | 68500 |
| W3278-T3U2.F16-1.ulgG4.SP | 58.9 | 6753 | >1000 | 55200 |
| W3278-U2T3,F18R-1.ulgG4.SP | 41.7 | 4077 | 2.5 | 66700 |
| W3278-U3T2.F18R-1.ulgG4.SP | 26.6 | 536 | 1.3 | 55400 |
| W3278-T3U2.F17R-1.ugG4.SP | 17.3 | 7355 | 85.3 | 64900 |
| anti-CD3 Fab (T2) | 33.0 | 3214 | | |
| Anti-CD3 Fab (T3) | 43.7 | 3803 | | |
| Anti-CD20.lgG4 (U2) | | | 11.4 | 53500 |
| Anti-CD20.lgG4 (U3) | | | 0.9 | 33300 |

For testing the simultaneous binding of W3278 BsAb to CD3 and CD20 expressing cells, 1×10⁶/ml Raji cells and 1×10⁶/ml Jurkat cells were labeled with 50 nM Calcein-AM (Invitrogen-C3099) and 20 nM FarRed (Invitrogen-C34572), respectively. After washing with cold 1% BSA/1×PBS, the labelled Raji and Jurkat cells were resuspended and mixed to a final concentration of 1×10⁶/ml at the ratio of 1:1. 1×10⁵/well of the mixed cells were plated and serial dilutions of W3278 BsAb was then added. After incubation at 4° C. for 60 minutes, the percentage of Calcein-AM and FarRed double positive cells was analyzed by FACS.

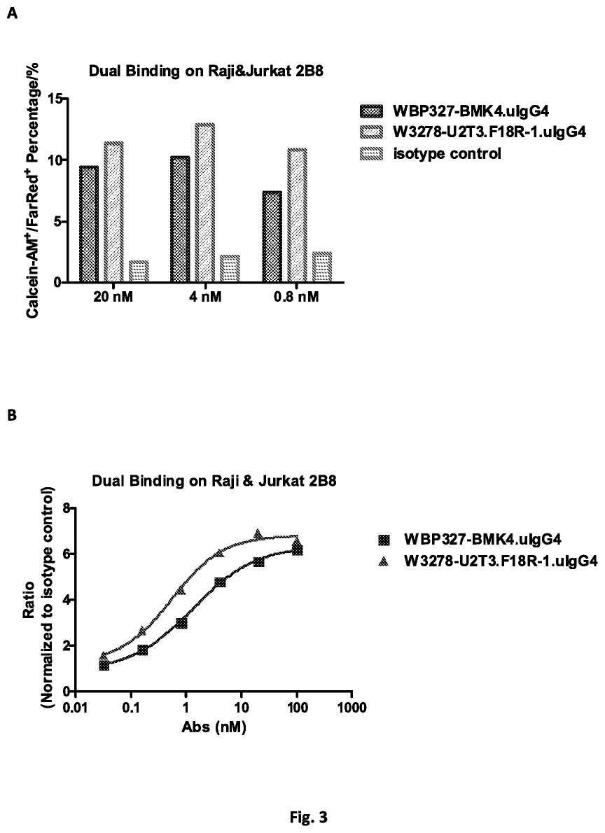

The results in FIG. 3 indicate that W3278 lead BsAb shows dose-dependent simultaneous dual target binding, which is more potent than that of BMK4.

### 3. In Vitro Cytotoxicity Assays

The efficacies of BsAb to mediate tumor cell lysis by CD8⁺ T lymphocytes were determined by FACS based cytotoxicity assay. Briefly, the freshly isolated human CD8⁺ T cells are cultured 3 to 5 days in complete media containing 50 IU/ml recombinant human IL-2 and 10 ng/ml OKT-3. At the following day, the target cells, Raji, Ramos, NAMALWA and SU-DHL-1 (1×10⁶ cells/ml) were labeled with 20 nM Far-Red (Invitrogen-C34572) in DPBS for 30 minutes at 37° C. and then washed twice with assay buffer (Phenol red free RPMI 1640 culture medium+10% FBS). Far-Red-labeled target cells (2×10⁴/well) were plated in 110 μl/well complete media containing effector CD8⁺ T cells (effector/target cell ratio 5:1) and serial dilution of BsAbs or hIgG4 isotype control and incubated at 37° C. for overnight. Finally, Propidium Iodide (PI) (Invitrogen-P3566) was added and incubated for 15 minutes at room temperature before analysis by flow cytometry. Percent cytotoxicity was calculated using the equation as: Cytotoxicity %=100*Far Red⁺PI⁺/(Far Red⁺Pt⁺Far Red⁺PI⁻)*100%. The EC₅₀ values of in vitro cytotoxicity were determined using Prism four-parameter non-linear regression analysis.

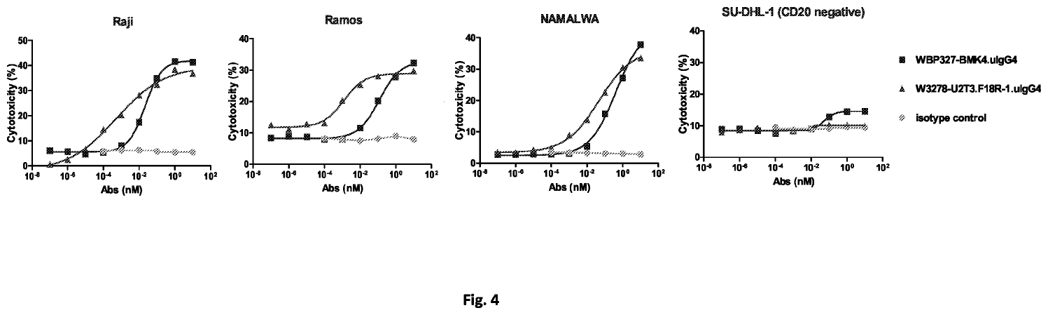

The results in FIG. 4 show that W3278 lead Ab “W3278-U2T3.F18R-1.uIgG4” does not mediate cell killing of CD20 negative SU-DHL-1 cells. W3278 lead Ab “W3278-U2T3.F18R-1.uIgG4” mediates cell killing of CD20 positive cells more potently than BMK4, and the killing EC50 are proportionally increased with cell surface CD20 expression levels. The BsAb mediated cytotoxicity EC50 and maximum cytotoxicity (Max Cyto) % are shown in Table 4.

TABLE 4
The cytotoxicity EC50 and maximum cytotoxicity % of different B cell lines by
W3278 lead BsAb and BMK4 BsAb.

| | Raji | | Ramos | | NAMALWA | | SU-DHL-1 | |
|---|---|---|---|---|---|---|---|---|
| Abs | EC50 (pM) | Max Cyto % | EC50 (pM) | Max Cyto % | EC50 (pM) | Max Cyto % | EC50 (pM) | Max Cyto % |
| WBP327-BMK4.uIgG4 | 21.49 | 36.4 | 119.2 | 24.6 | 411.7 | 40.3 | 53.59 | 6.1 |
| W3278-U2T3.F18R-1.uIgG4 | 0.52 | 41.7 | 1.25 | 17.1 | 46.8 | 33.0 | NA | 1.6 |

### 4. Cell Activation and Cytokine Release Assay

T cell activation mediated by BsAbs was determined by flow cytometry measuring the percentage of CD69 or CD25 expressing effector cells. Freshly isolated purified CD4⁺ T cells and CD8⁺ T cells were examined as effector cells, respectively. Briefly, 5×10⁴ CD4⁺ or CD8⁺ T cells were plated in 110 μl/well complete media containing serial dilution of BsAbs or hIgG4 isotype control antibody, in the presence 1×10⁴ Raji or SU-DHL-1 cells/well for 24 hours at 37° C. After incubation, the cells were washed twice with 1% BSA/1λDPBS and then stained with anti-human Ab panel (FITC labeled anti-human CD4 (BD Pharmingen-550628); PerCP-Cy5.5 labeled anti-human CD8 (BD Pharmingen-565310); PE labeled anti-human CD69 (BD Pharmingen-555531) and APC labeled anti-human CD25 (BD Pharmingen-555434)) at 4° C. for 30 minutes. T cell activation evaluated by CD69 or CD25 expression was analyzed by FACS. EC50 of T-cell activation was determined by using Prism four-parameter non-linear regression analysis.

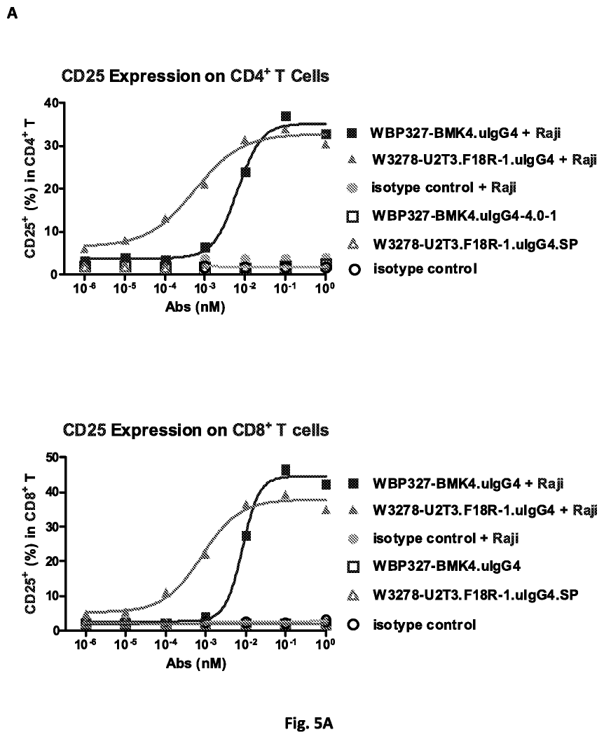

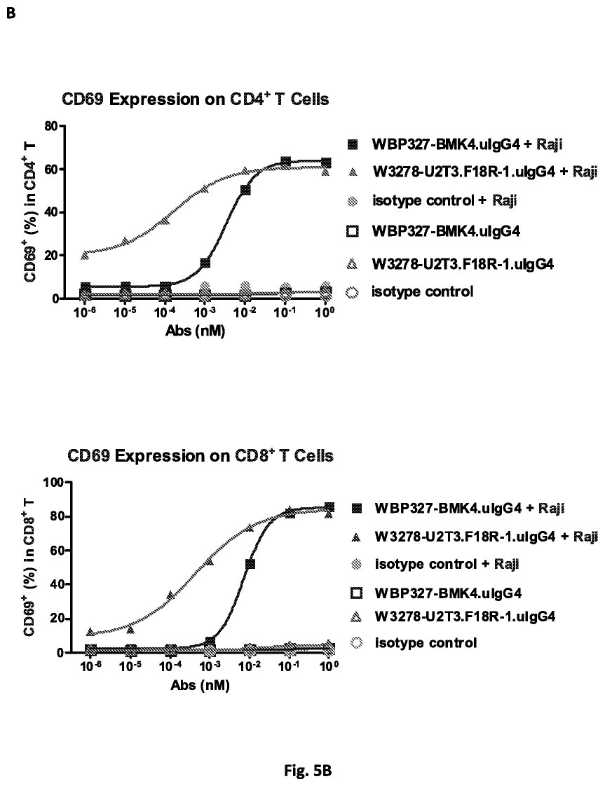

In the absence of target cells, W3278 lead Ab does not induce T cell activation. Only in the presence of target cells, W3278 lead Ab induces CD4+ and CD8+ T cell activation, shown by CD25 expression (FIG. 5A) and CD69 expression (FIG. 5B), and that is more potently than BMK4. The activation EC50s are shown in Tables 5A and 5B.

TABLE 5A
T cell CD69 expression EC50 mediated by W3278 BsAb and
BMK4 BsAb in the presence of Raji cells.

| | CD4⁺/Raji | | CD8⁺/Raji | |
|---|---|---|---|---|
| | EC50 | Max | EC50 | Max |
| Abs | (pM) | % | (pM) | % |
| W3278-U2T3.F18R-1.ulgG4.SP | 0.6 | 34.0 | 0.8 | 39.3 |
| WBP327-BMK4.ulgG4 | 6.5 | 37.1 | 8.2 | 46.5 |

TABLE 5B
T cell CD69 expression EC50 mediated by W3278 BsAb and
BMK4 BsAb in the presence of Raji cells.

| | CD4⁺/Raji | | CD8⁺/Raji | |
|---|---|---|---|---|
| | EC50 | Max | EC50 | Max |
| Abs | (pM) | % | (pM) | % |
| W3278-U2T3.F18R-1.ulgG4.SP | 0.2 | 61.8 | 0.4 | 83.8 |
| WBP327-BMK4.ulgG4 | 3.5 | 64.2 | 7.3 | 86.0 |

For cytokine release (TNF-α and IL-2) assay, 5×10⁴ freshly isolated CD4⁺ T cells were plated in 110 μl/well complete media containing serial dilution of BsAbs or hIgG4 isotype control antibody, in the presence of 1×10⁴ Raji or SU-DHL-1 cells/well for 24 hours at 37° C. After 24 hours incubation, the plates were centrifuged and supernatants were harvested stored at −80° C. for cytokine concentration measurement by ELISA.

For TNF-α detection by ELISA, 96-well ELISA plates (Nunc MaxiSorp, ThermoFisher) were coated with 50 μl of capture antibody purified anti-human TNF (BD Pharmingen-51-26371E) in Carbonate-bicarbonate buffer (20 mM Na₂CO₃, 180 mM NaHCO₃, pH9.2) overnight at 4° C. On the next day, plates were washed with washing buffer (1×PBST buffer, 0.05% Tween-20) and then blocked with 200 μl Assay Diluent (PBS+10% FBS). After blocking, 50 μl of testing samples and recombinant human TNF Standard (BD Pharmingen-51-26376E)) were added and the plates were incubated at room temperature for 2 hours. The binding of TNF-α to the plates was detected by the detection antibody biotinylated Anti-Human TNF (BD Pharmingen-51-26372E).

Streptavidin-HRP reagent (BD Pharmingen-51-9002813) and Tetramethylbenzidine (TMB) Substrate (Sigma-860336-5G) were used for the color reaction. Washing with washing buffer were applied between the steps. The color reaction was stopped after approximate 30 minutes by 2 M HCl. The absorbance of the wells was measured at 450 nm with a multiwall plate reader (SpectraMax® M5e). Similarly, IL-2 concentration in culture supernatants were measured by ELISA. Anti-human IL-2 Antibody mAb (R&D-MAB602) was used as capture antibody and biotinylated anti-human IL-2 antibody (R&D-BAF202) was used as detection antibody.

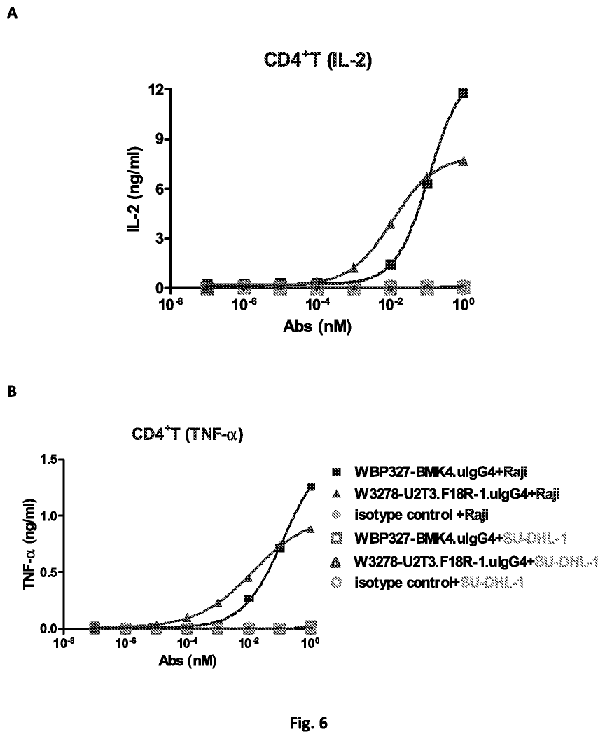

As shown in FIG. 6, in the presence of CD20 negative SU-DHL-1 cells, W3278 lead Ab does not induce T cell cytokine release. Only in the presence of target Raji cells, W3278 lead Ab can induce lower level of cytokine release, compared with that by BMK4. And the EC50 window, indicated by the EC50 ratio between cytokine release and cell killing, is larger by W3278 Ab than by BMK4 (Table 6).

TABLE 6
BsAb mediated CD4⁺ T cell cytokine release EC50 and max level, as well as EC50
ratio of cytokine release to Raji cell killing.

| | TNF-a | | IL-2 | | EC50 ratio | EC50 ratio |
|---|---|---|---|---|---|---|
| | EC50 | Max level | EC50 | Max level | TNFa release/ | IL-2 release/Raji |
| Ab | (pM) | (ng/ml) | (pM) | (ng/ml) | Raji cell killing | cell killing |
| WBP327-BMK4.uIgG4 | 131.4 | 1.3 | 113.6 | 11.8 | 6 | 5 |
| | | | | | (131.4 pM/ | (113.6 pM/ |
| | | | | | 21.49 pM) | 21.49 pM) |
| W3278-U2T3.F18R- | 13.5 | 0.9 | 11.4 | 7.7 | 26 | 22 |
| 1.uIgG4.SP | | | | | (13.5 pM/ | (11.4 pM/ |
| | | | | | 0.52 pM) | 0.52 pM) |

### 5. Serum Stability Test

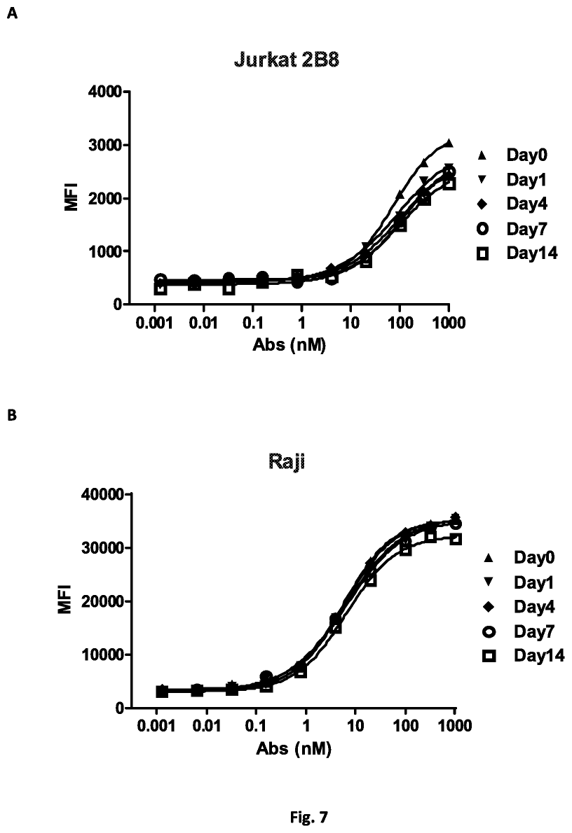

Antibodies were gently mixed with freshly collected human serum and >95% of serum proportion in the mixed sample was ensured. The mixed sample aliquots were incubated at 37° C. for 0 to 14 days. At each time point as shown in FIG. 7, the samples were quickly-frozen in liquid nitrogen and stored at −80° C. until analysis. The bindings of each sample to Raji or Jurkat cells were analyzed by FACS.

As shown in FIG. 7, the bindings of human serum treated W3278 Ab to both Jurkat (FIG. 7A) and Raji (FIG. 7B) cells were similar as that of fresh thawed Ab (day 0). These results suggested that W3278 Ab was stable in human serum for at least 14 days (FIG. 7).

### 6. DSF Assay and Thermal Stability Test

A DSF assay was performed using Real-Time Fluorescent Quantitative PCR (QuantStudio 7 Flex, Thermo Fisher Scientific). Briefly, 19 μL of antibody solution was mixed with 1 μL of 62.5×SYPRO Orange solution (Invitrogen) and added to a 96 well plate (Biosystems). The plate was heated from 26° C. to 95° C. at a rate of 2° C./min, and the resulting fluorescence data were collected. The negative derivatives of the fluorescence changes with respect to different temperatures were calculated, and the maximal value was defined as melting temperature Tₕ. If a protein has multiple unfolding transitions, the first two Tₕ were reported, named as Tₘ₁ and Tₘ₂. The Tₘ₁ is always interpreted as the formal melting temperature Tₘ to facilitate comparisons between different proteins. Data collection and Tₕ calculation were conducted automatically by operation software (QuantStudio Real-Time PCR PCR Software v1.3). The Tₘ₁ and Tₘ₂ values of W3278 Ab in different buffers were shown in table 7. The Tₘ of W3278 is about 61° C., indicating a good thermal stability of W3278 Ab.

TABLE 7
Thermal stability by DSF test. (The Tm value is indicated by Tm1
in the table)

| | | | Tm1 | Tm2 |
|---|---|---|---|---|
| Protein Name | pI | Buffer | (° C.) | (° C.) |
| W3278- | 7.42 | PBS | 61.1 | 72.6 |
| U2T3.F18R- | | 20 mM Histidine + 7% | 60.3 | 73.6 |
| 1.ulgG4.SP | | Sucrose pH 6.5 | | |
| | | 50 mM NaAC + 7% | 61.0 | 74.2 |
| | | Sucrose pH 5.6 | | |

### Example 4

### In Vivo Antitumor Efficacy

The antibody in vivo antitumor efficacy was tested in an admixed PBMC humanized model bearing Raji tumor in NOG mice. Female NOG mice (Beijing Vital River Laboratory Animal Technology Co., LTD) of 6-8 week-old were used in the studies. The Raji tumor cells (ATCC® CCL-86™) were maintained in vitro as a monolayer culture in 1640 medium supplemented with 10% fetal bovine serum, 100 U/ml penicillin and 100 μg/ml streptomycin at 37° C. in an atmosphere of 5% CO₂ in air. The tumor cells were routinely sub-cultured twice weekly. The cells growing in an exponential growth phase were harvested and counted for tumor inoculation. Human PBMCs were isolated from heparin whole blood of a single healthy donor by using Ficoll-Paque Plus per manufacturer's instructions.

For therapeutic model, each mouse was co-inoculated subcutaneously at the right upper flank with pre-mixed Raji tumor cells (2.0×10⁶) and PBMC (3.0×10⁶). When the average tumor volume reached approximately to 60 mm³, the animals were randomized for grouping and received the first antibody injection. For the efficacy study the mice were treated with indicated amount of antibodies intravenously twice weekly for 3 weeks. All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). For all tumor studies, mice were weighed and tumor growth was measured twice a week using calipers. Tumor volume was estimated as ½(length×width²).

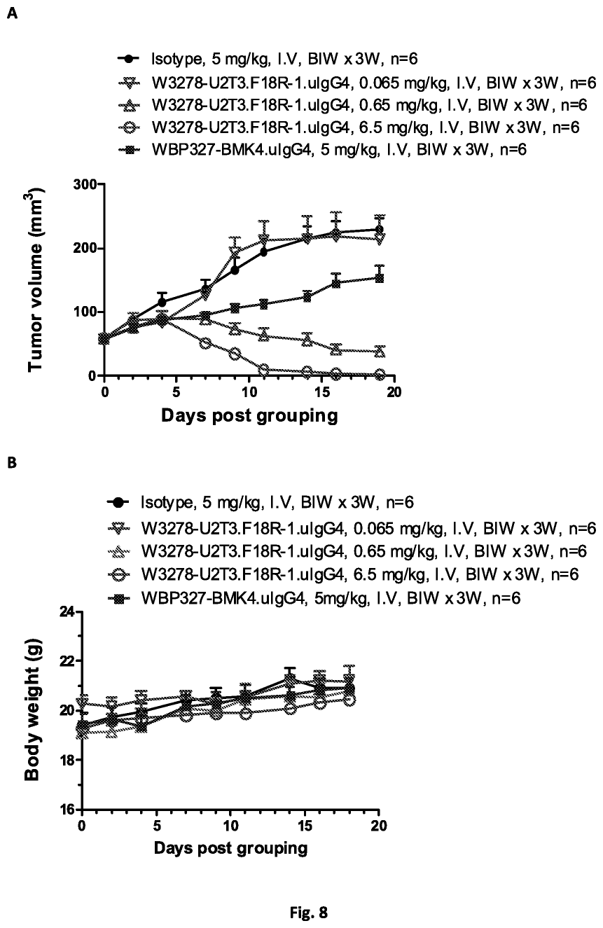

As shown in FIG. 8, W3278 lead Ab treatment displays dose-dependent antitumor activity, which is more potent compared to BMK4 (FIG. 8A). The mouse body weights are normal during the experiment (FIG. 8B).

### Example 5

### A Single Dose Study of WBP3278 BsAb in Naïve Cynomolgus Monkeys

To determine whether a treatment with WBP3278 bispecific antibody could deplete circulating B cells in primates, and whether it resulted in any unexpected toxicity, the inventor conducted an exploratory non-GLP pharmacology study in cynomolgus monkeys (Macaca Fascicularis). 4 naïve male cynomolgus monkeys, age of 3-4 years old, weight of about 4 kg, were supplied by Guangdong Zhao Qing Chuang Yao Biotechnology Co., Ltd. All the procedures related to animal handling, care and the treatment in the study were performed according to the guidelines approved by the Institutional Animal Care and Use Committee (IACUC) of PharmaLegacy Laboratories following the guidance of the Association for Assessment and Accreditation of Laboratory Animal Care (AAALAC).

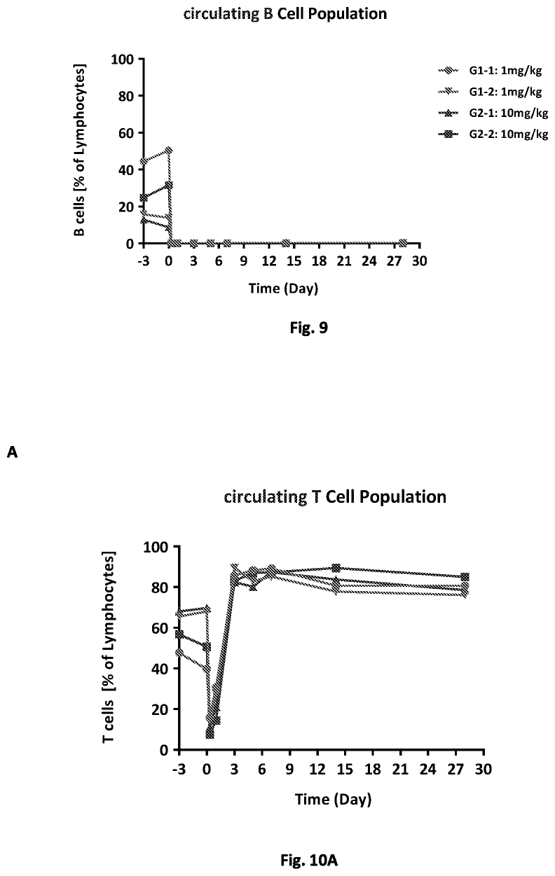

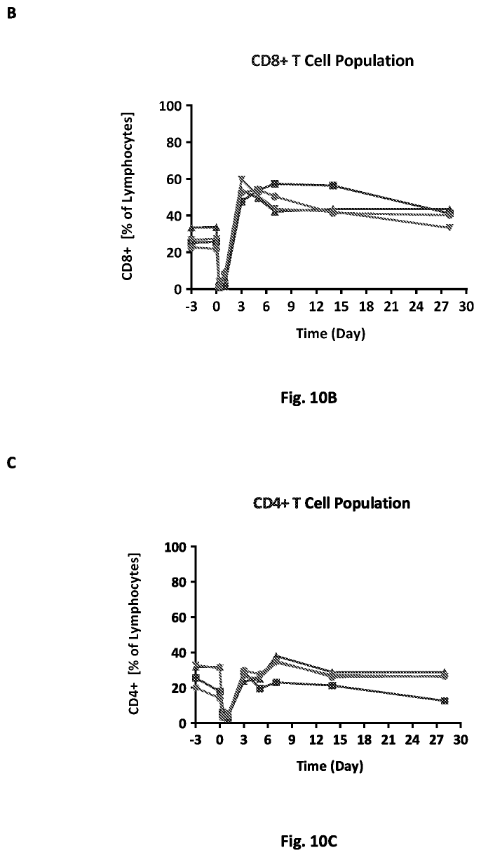

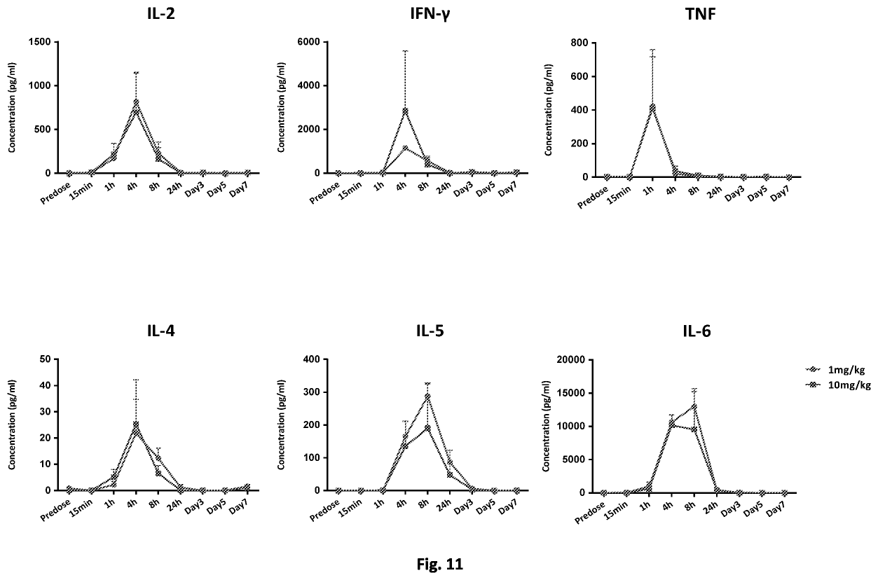

Four animals in 2 groups (2 animals/group) were administered with WBP3278 lead bispecific antibody (i.e., W3278-U2T3.F18R-1.uIgG4, referred to as WBP3278 lead Ab hereafter) at 1 mg/kg (Group 1) and 10 mg/kg (Group 2) once by slow i.v. injection within 60 s, respectively. The levels of circulating B and T cells in peripheral blood were monitored for 4 weeks by FACS for B lymphocytes (CD45+/CD20+); T lymphocytes (CD45+/CD3+); CD4+T lymphocytes (CD4+/CD45+/CD3+) and CD8+T lymphocytes (CD8+/CD45+/CD3+). The levels of circulating inflammatory cytokines were analyzed by using BD™ Cytometric Bead Array (CBA) Non-Human Primate Th1/Th2 Cytokine Kit (BD Bioscience, Cat: 557800). Treatment with WBP3278 lead Ab resulted in an immediate and complete depletion of circulating B cells, persisting for at least 4 weeks (FIG. 9). Levels of circulating T cells were initially also reduced following WBP3278 lead Ab treatment, and returned to baseline or slightly higher level after 72 hours and persisted for at least 4 weeks (FIG. 10A). The level of CD8+ T cells (FIG. 10B) was increased and that of CD4+ T cells (FIG. 10C) were returned to normal level after 72 hours and persisted for at least 4 weeks. A rapid rise in levels of circulating cytokines was observed following treatment with WBP3278 lead Ab, and all cytokines returned to normal levels after 24 hours (FIG. 11).

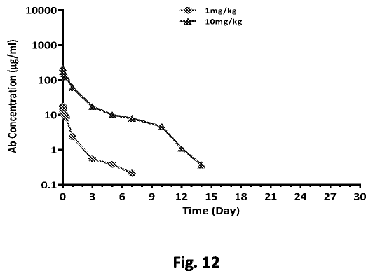

The concentrations of WBP3278 lead Ab in serum were determined by ELISA. Briefly, ELISA plates were coated with anti-Human IgG (SouthernBiotech, #2049-01), then serial dilutions of serum samples were added. The binding signals were detected with Goat anti-Human IgG-Biotin (SouthernBiotech, #2049-08) and Streptavidin-HRP (Life, #SNN1004). The absorbance was measured at (450-540) nm with a multiwell plate reader (SpectraMax® M5e). The serum concentration of WBP3278 lead Ab in monkeys was subjected to a non-compartmental pharmacokinetic analysis by using the Phoenix WinNonlin software (version 8.1, Pharsight, Mountain View, Calif.). The linear/log trapezoidal rule was applied in obtaining the PK parameters. Individual BLQ was excluded from the calculation of the mean concentrations. The nominal dose levels and nominal sampling times were used in the calculation of all pharmacokinetic parameters. The summary for PK parameters was listed in Table 8 and FIG. 12. In conclusion, the systemic exposure of Co was increased from 19.9 μg/mL to 282 μg/mL (about 14-fold) and AUC₀₋ₗₐₛₜ was increased from 259 μg·h/mL to 5788 μg·h/mL (about 22-fold) as the dosage increased from 1 to 10 mg/kg. The serum half-life (T_{1/2}) of WBP3278 lead Ab was about 43.3 hours and 89.8 hours at 1 and 10 mg/kg, respectively.

TABLE 8
Summary of PK parameters of WBP3278 lead Ab

| PK parameters | 1 mg/kg (Mean) | 10 mg/kg (Mean) |
|---|---|---|
| C₀ (μg/mL) | 19.9 | 282 |
| T_{1/2} (h) | 43.3 | 89.8 |
| AUC₀₋ₗₐₛₜ (μg.h/mL) | 259 | 5788 |

Furthermore, cage-side observations during the experiment revealed no unexpected toxicities for WBP3278 lead Ab at both high and low dose levels (data not shown).

The results from this experiment demonstrated that WBP3278 lead Ab was capable of effectively depeleting B cells in vivo, without adverse reactions such as cytokine storm and the like, and it had sufficient serum half-life (T_{1/2}) in cynomolgus monkeys. These experiment results provided support for advancing pre-clinical developments of WBP3278 lead Ab.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. A bispecific polypeptide complex, comprising a first antigen-binding moiety associated with a second antigen-binding moiety, wherein:
the first antigen-binding moiety comprises:
a first polypeptide comprising, from N-terminus to C-terminus, a first heavy chain variable domain (VH) of a first antibody operably linked to a first T cell receptor (TCR) constant region (C1), and
a second polypeptide comprising, from N-terminus to C-terminus, a first light chain variable domain (VL) of the first antibody operably linked to a second TCR constant region (C2),
wherein:
C1 and C2 are capable of forming a dimer comprising at least one non-native interchain bond between C1 and C2, and the non-native interchain bond is capable of stabilizing the dimer
and
the second antigen-binding moiety comprises:
a second heavy chain variable domain (VH2) of a second antibody operably linked to an antibody heavy chain CH1 domain, and
a second light chain variable domain (VL2) of the second antibody operably linked to an antibody light chain constant (CL) domain,
wherein:
one of the first and the second antigen-binding moiety is an anti-CD3 binding moiety, and the other one is an anti-CD20 binding moiety,
the anti-CD3 binding moiety is derived from an anti-CD3 antibody comprising:
a) a heavy chain CDR1 comprising an amino acid sequence selected from the group consisting of SEQ ID NO: 25, 1, 13, 37 and 49,
b) a heavy chain CDR2 comprising an amino acid sequence selected from the group consisting of SEQ ID NO: 26, 2, 14, 38 and 50,
c) a heavy chain CDR3 comprising an amino acid sequence selected from the group consisting of SEQ ID NO: 27, 3, 15, 39 and 51,
d) a kappa light chain CDR1 comprising an amino acid sequence selected from the group consisting of SEQ ID NO: 28, 4, 16, 40 and 52,
e) a kappa light chain CDR2 comprising an amino acid sequence selected from the group consisting of SEQ ID NO: 29, 5, 17, 41 and 53, and
f) a kappa light chain CDR3 comprising an amino acid sequence selected from the group consisting of SEQ ID NO: 30, 6, 18, 42 and 54,
the anti-CD20 binding moiety is derived from an anti-CD20 antibody comprising:
a) a heavy chain CDR1 comprising an amino acid sequence selected from the group consisting of SEQ ID NO: 31, 7, 19, 43 and 55,
b) a heavy chain CDR2 comprising an amino acid sequence selected from the group consisting of SEQ ID NO: 32, 8, 20, 44 and 56,
c) a heavy chain CDR3 comprising an amino acid sequence selected from the group consisting of SEQ ID NO: 33, 9, 21, 45 and 57,
d) a kappa light chain CDR1 comprising an amino acid sequence selected from the group consisting of SEQ ID NO: 34, 10, 22, 46 and 58,
e) a kappa light chain CDR2 comprising an amino acid sequence selected from the group consisting of SEQ ID NO: 35, 11, 23, 47 and 59, and
f) a kappa light chain CDR3 comprising an amino acid sequence selected from the group consisting of SEQ ID NO: 36, 12, 24, 48 and 60.

2. The bispecific polypeptide complex of claim 1, wherein the anti-CD3 binding moiety comprises a heavy chain variable domain sequence comprising an amino acid sequence selected from the group consisting of SEQ ID NO: 65, 61, 63, 67 and 69 and a light chain variable domain sequence comprising an amino acid sequence selected from the group consisting of SEQ ID NO: 66, 62, 64, 68 and 70.

3. The bispecific polypeptide complex of claim 1, wherein the anti-CD20 binding moiety comprises a heavy chain variable domain sequence comprising SEQ ID NO: 75, 71, 73, 77 and 79 and a light chain variable domain sequence comprising SEQ ID NO: 76, 72, 74, 78 and 80.

4. The bispecific polypeptide complex of claim 1, wherein the first antigen-binding moiety is linked to a first dimerization domain, and the second antigen-binding moiety is linked to a second dimerization domain, wherein the first and the second dimerization domains are associated via a connecter, a disulphide bond, a hydrogen bond, electrostatic interaction, a salt bridge, or hydrophobic-hydrophilic interaction, or a combination thereof.

5. (canceled)

6. The bispecific polypeptide complex of claim 4, wherein the first and/or the second dimerization domain comprises at least a portion of an antibody hinge region derived from IgG1, IgG2 or IgG4.

7. The bispecific polypeptide complex of claim 6, wherein the first and/or the second dimerization domain comprises and antibody CH2 domain, and/or an antibody CH3 domain.

8. The bispecific polypeptide complex of claim 6, wherein the first dimerization domain is operably linked to the first TCR constant region (C1) at a third conjunction domain; and/or wherein the second dimerization domain is operably linked to the heavy chain variable domain of the second antigen-binding moiety.

9. (canceled)

10. The bispecific polypeptide complex of claim 4, wherein the first and the second dimerization domains are different and associate in a way that discourages homodimerization and/or favors heterodimerization.

11. The bispecific polypeptide complex of claim 10, wherein the first and the second dimerization domains are capable of associating into heterodimers via knobs-into-holes, hydrophobic interaction, electrostatic interaction, hydrophilic interaction, or increased flexibility.

12. The bispecific polypeptide complex of claim 1, wherein the bispecific polypeptide complex comprises a combination of four polypeptide sequences: SEQ ID NO: 89, SEQ ID NO: 90, SEQ ID NO: 91, and SEQ ID NO: 92; or
wherein the bispecific polypeptide complex comprises a combination of four polypeptide sequences: SEQ ID NO: 81, SEQ ID NO: 82, SEQ ID NO: 83, and SEQ ID NO: 84; or
wherein the bispecific polypeptide complex comprises a combination of four polypeptide sequences: SEQ ID NO: 85, SEQ ID NO: 86, SEQ ID NO: 87, and SEQ ID NO: 88; or
wherein the bispecific polypeptide complex comprises a combination of four polypeptide sequences: SEQ ID NO: 93, SEQ ID NO: 94, SEQ ID NO: 95, and SEQ ID NO: 96; or
wherein the bispecific polypeptide complex comprises a combination of four polypeptide sequences: SEQ ID NO: 97, SEQ ID NO: 98, SEQ ID NO: 99, and SEQ ID NO: 100.

13-16. (canceled)

17. The bispecific polypeptide complex of claim 12, wherein one or more amino acids at positions 182, 193, 203, 206 and 207 in the polypeptide sequence of SEQ ID NO: 92 are modified to be any amino acid other than Ser and Thr so that the glycosylation site is removed.

18. The bispecific polypeptide complex of claim 17, wherein the amino acid at position 193 in the polypeptide sequence of SEQ ID NO: 92 is modified to be Ala, Gly, Pro or Val.

19. A conjugate comprising the bispecific polypeptide complex of claim 1, conjugated to a moiety.

20. An isolated polynucleotide encoding the bispecific polypeptide complex of claim 1.

21. An isolated vector comprising the polynucleotide of claim 20.

22. A host cell comprising the isolated polynucleotide of claim 20 or an isolated vector comprising the polynucleotide of claim 20.

23. A method of expressing the bispecific polypeptide complex of claim 1, comprising culturing a host cell comprising an isolated polynucleotide encoding the bispecific polypeptide complex of claim 1 under the condition at which the bispecific polypeptide complex is expressed, and isolating the bispecific polypeptide complex.

24-26. (canceled)

27. A pharmaceutical composition comprising the bispecific polypeptide complex of claim 1 and a pharmaceutically acceptable carrier.

28. A method of treating a CD20-related disease or condition in a subject in need thereof, comprising administrating to the subject a therapeutically effective amount of the bispecific polypeptide complex of claim 1, wherein the CD20-related disease or condition is cancer selected from lymphoma, lung cancer, liver cancer, cervical cancer, colon cancer, breast cancer, ovarian cancer, pancreatic cancer, melanoma, glioblastoma, prostate cancer, esophageal cancer or gastric cancer.

29-31. (canceled)

32. A kit comprising the bispecific polypeptide complex of claim 1.

33. (canceled)


SEQUENCE LISTING (122 sequences)

SEQ ID NO: 1 - protein, 10 aa, Artificial Sequence
  CDR1
GYSFTTYYIH

SEQ ID NO: 2 - protein, 17 aa, Artificial Sequence
  CDR2
WIFPGNDNIKYSEKFKG

SEQ ID NO: 3 - protein, 10 aa, Artificial Sequence
  CDR3
DSVSIYYFDY

SEQ ID NO: 4 - protein, 17 aa, Artificial Sequence
  CDR1
KSSQSLLNSRTRKNYLA

SEQ ID NO: 5 - protein, 7 aa, Artificial Sequence
  CDR2
WASTRKS

SEQ ID NO: 6 - protein, 8 aa, Artificial Sequence
  CDR3
TQSFILRT

SEQ ID NO: 7 - protein, 10 aa, Artificial Sequence
  CDR1
GFTFNDYAMH

SEQ ID NO: 8 - protein, 17 aa, Artificial Sequence
  CDR2
TISWNSGSIGYADSVKG

SEQ ID NO: 9 - protein, 13 aa, Artificial Sequence
  CDR3
DIQYGNYYYGMDV

SEQ ID NO: 10 - protein, 11 aa, Artificial Sequence
  CDR1
RASQSVSSYLA

SEQ ID NO: 11 - protein, 7 aa, Artificial Sequence
  CDR2
DASNRAT

SEQ ID NO: 12 - protein, 9 aa, Artificial Sequence
  CDR3
QQRSNWPIT

SEQ ID NO: 13 - protein, 10 aa, Artificial Sequence
  CDR1
GFAFTDYYIH

SEQ ID NO: 14 - protein, 17 aa, Artificial Sequence
  CDR2
WISPGNVNTKYNENFKG

SEQ ID NO: 15 - protein, 10 aa, Artificial Sequence
  CDR3
DGYSLYYFDY

SEQ ID NO: 16 - protein, 17 aa, Artificial Sequence
  CDR1
KSSQSLLNSRTRKNYLA

SEQ ID NO: 17 - protein, 7 aa, Artificial Sequence
  CDR2
WASTRQS

SEQ ID NO: 18 - protein, 8 aa, Artificial Sequence
  CDR3
TQSHTLRT

SEQ ID NO: 19 - protein, 10 aa, Artificial Sequence
  CDR1
GYTFTSYNMH

SEQ ID NO: 20 - protein, 17 aa, Artificial Sequence
  CDR2
AIYPGNGDTSYNQKFKG

SEQ ID NO: 21 - protein, 12 aa, Artificial Sequence
  CDR3
STYYGGDWYFNV

SEQ ID NO: 22 - protein, 10 aa, Artificial Sequence
  CDR1
RASSSVSYIH

SEQ ID NO: 23 - protein, 7 aa, Artificial Sequence
  CDR2
ATSNLAS

SEQ ID NO: 24 - protein, 9 aa, Artificial Sequence
  CDR3
QQWTSNPPT

SEQ ID NO: 25 - protein, 10 aa, Artificial Sequence
  CDR1
GFAFTDYYIH

SEQ ID NO: 26 - protein, 17 aa, Artificial Sequence
  CDR2
WISPGNVNTKYNENFKG

SEQ ID NO: 27 - protein, 10 aa, Artificial Sequence
  CDR3
DGYSLYYFDY

SEQ ID NO: 28 - protein, 17 aa, Artificial Sequence
  CDR1
KSSQSLLNSRTRKNYLA

SEQ ID NO: 29 - protein, 7 aa, Artificial Sequence
  CDR2
WASTRQS

SEQ ID NO: 30 - protein, 8 aa, Artificial Sequence
  CDR3
TQSHTLRT

SEQ ID NO: 31 - protein, 10 aa, Artificial Sequence
  CDR1
GYTFTSYNMH

SEQ ID NO: 32 - protein, 17 aa, Artificial Sequence
  CDR2
AIYPGNGDTSYNQKFKG

SEQ ID NO: 33 - protein, 12 aa, Artificial Sequence
  CDR3
STYYGGDWYFNV

SEQ ID NO: 34 - protein, 10 aa, Artificial Sequence
  CDR1
RASSSVSYIH

SEQ ID NO: 35 - protein, 7 aa, Artificial Sequence
  CDR2
ATSNLAS

SEQ ID NO: 36 - protein, 9 aa, Artificial Sequence
  CDR3
QQWTSNPPT

SEQ ID NO: 37 - protein, 10 aa, Artificial Sequence
  CDR1
GYSFTTYYIH

SEQ ID NO: 38 - protein, 17 aa, Artificial Sequence
  CDR2
WIFPGNDNIKYSEKFKG

SEQ ID NO: 39 - protein, 10 aa, Artificial Sequence
  CDR3
DSVSIYYFDY

SEQ ID NO: 40 - protein, 17 aa, Artificial Sequence
  CDR1
KSSQSLLNSRTRKNYLA

SEQ ID NO: 41 - protein, 7 aa, Artificial Sequence
  CDR2
WASTRKS

SEQ ID NO: 42 - protein, 8 aa, Artificial Sequence
  CDR3
TQSFILRT

SEQ ID NO: 43 - protein, 10 aa, Artificial Sequence
  CDR1
GFTFNDYAMH

SEQ ID NO: 44 - protein, 17 aa, Artificial Sequence
  CDR2
TISWNSGSIGYADSVKG

SEQ ID NO: 45 - protein, 13 aa, Artificial Sequence
  CDR3
DIQYGNYYYGMDV

SEQ ID NO: 46 - protein, 11 aa, Artificial Sequence
  CDR1
RASQSVSSYLA

SEQ ID NO: 47 - protein, 7 aa, Artificial Sequence
  CDR2
DASNRAT

SEQ ID NO: 48 - protein, 9 aa, Artificial Sequence
  CDR3
QQRSNWPIT

SEQ ID NO: 49 - protein, 10 aa, Artificial Sequence
  CDR1
GFAFTDYYIH

SEQ ID NO: 50 - protein, 17 aa, Artificial Sequence
  CDR2
WISPGNVNTKYNENFKG

SEQ ID NO: 51 - protein, 10 aa, Artificial Sequence
  CDR3
DGYSLYYFDY

SEQ ID NO: 52 - protein, 17 aa, Artificial Sequence
  CDR1
KSSQSLLNSRTRKNYLA

SEQ ID NO: 53 - protein, 7 aa, Artificial Sequence
  CDR2
WASTRQS

SEQ ID NO: 54 - protein, 8 aa, Artificial Sequence
  CDR3
TQSHTLRT

SEQ ID NO: 55 - protein, 10 aa, Artificial Sequence
  CDR1
GYTFTSYNMH

SEQ ID NO: 56 - protein, 17 aa, Artificial Sequence
  CDR2
AIYPGNGDTSYNQKFKG

SEQ ID NO: 57 - protein, 12 aa, Artificial Sequence
  CDR3
STYYGGDWYFNV

SEQ ID NO: 58 - protein, 10 aa, Artificial Sequence
  CDR1
RASSSVSYIH

SEQ ID NO: 59 - protein, 7 aa, Artificial Sequence
  CDR2
ATSNLAS

SEQ ID NO: 60 - protein, 9 aa, Artificial Sequence
  CDR3
QQWTSNPPT

SEQ ID NO: 61 - protein, 119 aa, Artificial Sequence
  VH
QVQLVQSGAEVKKPGSSVKVSCKASGYSFTTYYIHWVRQAPGQGLEWMGWIFPGNDNIKYSEKFKGRVTITADKSTSTAYMELSSLRSEDTAVYYCAIDSVSIYYFDYWGQGTLVTVSS

SEQ ID NO: 62 - protein, 112 aa, Artificial Sequence
  VK
DIVMTQSPDSLAVSLGERATINCKSSQSLLNSRTRKNYLAWYQQKPGQPPKLLIYWASTRKSGVPDRFSGSGSGTDFTLTISSLQAEDVAVYYCTQSFILRTFGGGTKVEIK

SEQ ID NO: 63 - protein, 119 aa, Artificial Sequence
  VH
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSS

SEQ ID NO: 64 - protein, 112 aa, Artificial Sequence
  VK
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIK

SEQ ID NO: 65 - protein, 119 aa, Artificial Sequence; identical to SEQ ID NO: 63
  VH

SEQ ID NO: 66 - protein, 112 aa, Artificial Sequence; identical to SEQ ID NO: 64
  VK

SEQ ID NO: 67 - protein, 119 aa, Artificial Sequence; identical to SEQ ID NO: 61
  VH

SEQ ID NO: 68 - protein, 112 aa, Artificial Sequence; identical to SEQ ID NO: 62
  VK

SEQ ID NO: 69 - protein, 119 aa, Artificial Sequence; identical to SEQ ID NO: 63
  VH

SEQ ID NO: 70 - protein, 112 aa, Artificial Sequence; identical to SEQ ID NO: 64
  VK

SEQ ID NO: 71 - protein, 122 aa, Artificial Sequence
  VH
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 72 - protein, 107 aa, Artificial Sequence
  VK
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 73 - protein, 121 aa, Artificial Sequence [sequence omitted]

SEQ ID NO: 74 - protein, 106 aa, Artificial Sequence [sequence omitted]

SEQ ID NO: 75 - protein, 121 aa, Artificial Sequence [sequence omitted]

SEQ ID NO: 76 - protein, 106 aa, Artificial Sequence [sequence omitted]

SEQ ID NO: 77 - protein, 122 aa, Artificial Sequence; identical to SEQ ID NO: 71
  VH

SEQ ID NO: 78 - protein, 107 aa, Artificial Sequence; identical to SEQ ID NO: 72
  VK

SEQ ID NO: 79 - protein, 121 aa, Artificial Sequence [sequence omitted]

SEQ ID NO: 80 - protein, 106 aa, Artificial Sequence [sequence omitted]

SEQ ID NO: 81 - protein, 445 aa, Artificial Sequence
  heavy chain 1
QVQLVQSGAEVKKPGSSVKVSCKASGYSFTTYYIHWVRQAPGQGLEWMGWIFPGNDNIKYSEKFKGRVTITADKSTSTAYMELSSLRSEDTAVYYCAIDSVSIYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 82 - protein, 474 aa, Artificial Sequence
  heavy chain 2
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 83 - protein, 219 aa, Artificial Sequence
  light chain 1
DIVMTQSPDSLAVSLGERATINCKSSQSLLNSRTRKNYLAWYQQKPGQPPKLLIYWASTRKSGVPDRFSGSGSGTDFTLTISSLQAEDVAVYYCTQSFILRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 202 aa, Artificial Sequence
  light chain 2
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 85 - protein, 471 aa, Artificial Sequence
  heavy chain 1
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 86 - protein, 676 aa, Artificial Sequence
  heavy chain 2
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 87 - protein, 207 aa, Artificial Sequence
  light chain 1
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 88 - protein, 213 aa, Artificial Sequence
  light chain 2
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 700 aa, Artificial Sequence
  heavy chain 1
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSQVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 90 - protein, 447 aa, Artificial Sequence
  heavy chain 2
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 91 - protein, 213 aa, Artificial Sequence; identical to SEQ ID NO: 88
  light chain 1

SEQ ID NO: 92 - protein, 207 aa, Artificial Sequence; identical to SEQ ID NO: 87
  light chain 2

SEQ ID NO: 93 - protein, 701 aa, Artificial Sequence
  heavy chain 1
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSQVQLVQSGAEVKKPGSSVKVSCKASGYSFTTYYIHWVRQAPGQGLEWMGWIFPGNDNIKYSEKFKGRVTITADKSTSTAYMELSSLRSEDTAVYYCAIDSVSIYYFDYWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 94 - protein, 448 aa, Artificial Sequence
  heavy chain 2
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 95 - protein, 214 aa, Artificial Sequence
  light chain 1
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 207 aa, Artificial Sequence
  light chain 2
DIVMTQSPDSLAVSLGERATINCKSSQSLLNSRTRKNYLAWYQQKPGQPPKLLIYWASTRKSGVPDRFSGSGSGTDFTLTISSLQAEDVAVYYCTQSFILRTFGGGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 97 - protein, 703 aa, Artificial Sequence
  heavy chain 1
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 98 - protein, 447 aa, Artificial Sequence; identical to SEQ ID NO: 90
  heavy chain 2

SEQ ID NO: 99 - protein, 207 aa, Artificial Sequence; identical to SEQ ID NO: 87
  light chain 1

SEQ ID NO: 100 - protein, 213 aa, Artificial Sequence; identical to SEQ ID NO: 88
  light chain 2

SEQ ID NO: 101 - DNA, 357 bp, Artificial Sequence
  VH
caggtgcaactcgtgcagtctggagctgaagtgaagaagcctgggtcttcagtcaaggtcagttgcaaggccagtgggtattccttcactacctactacatccactgggtgcggcaggcaccaggacaggggcttgagtggatgggctggatctttcccggcaacgataatattaagtacagcgagaagttcaaagggagggtcaccattaccgccgacaaatccacttccacagcctacatggagttgagcagcctgagatccgaggatacagccgtgtactactgtgccattgacagcgtgtccatctactactttgactactggggccagggcacactggtcacagtgagcagc

SEQ ID NO: 102 - DNA, 336 bp, Artificial Sequence
  VK
gacatcgtcatgacccagtccccagactctttggcagtgtctctcggggaaagagctaccatcaactgcaagagcagccagtcccttctgaacagcaggaccaggaagaattacctcgcctggtaccaacagaagcccggacagcctcctaagctcctgatctactgggcctcaacccggaagagtggagtgcccgatcgctttagcgggagcggctccgggacagatttcacactgacaatttcctccctgcaggccgaggacgtcgccgtgtattactgtactcagagcttcattctgcggacatttggcggcgggactaaagtggagattaag

SEQ ID NO: 103 - DNA, 357 bp, Artificial Sequence
  VH
caggtgcagcttgtgcagtctggggcagaagtgaagaagcctgggtctagtgtcaaggtgtcatgcaaggctagcgggttcgcctttactgactactacatccactgggtgcggcaggctcccggacaagggttggagtggatgggatggatctccccaggcaatgtcaacacaaagtacaacgagaacttcaaaggccgcgtcaccattaccgccgacaagagcacctccacagcctacatggagctgtccagcctcagaagcgaggacactgccgtctactactgtgccagggatgggtactccctgtattactttgattactggggccagggcacactggtgacagtgagctcc

SEQ ID NO: 104 - DNA, 336 bp, Artificial Sequence
  VK
gatatcgtgatgacccagagcccagactcccttgctgtctccctcggcgaaagagcaaccatcaactgcaagagctcccaaagcctgctgaactccaggaccaggaagaattacctggcctggtatcagcagaagcccggccagcctcctaagctgctcatctactgggcctccacccggcagtctggggtgcccgatcggtttagtggatctgggagcgggacagacttcacattgacaattagctcactgcaggccgaggacgtggccgtctactactgtactcagagccacactctccgcacattcggcggagggactaaagtggagattaag

SEQ ID NO: 105 - DNA, 357 bp, Artificial Sequence; identical to SEQ ID NO: 103
  VH

SEQ ID NO: 106 - DNA, 336 bp, Artificial Sequence; identical to SEQ ID NO: 104
  VK

SEQ ID NO: 107 - DNA, 357 bp, Artificial Sequence; identical to SEQ ID NO: 101
  VH

SEQ ID NO: 108 - DNA, 336 bp, Artificial Sequence; identical to SEQ ID NO: 102
  VK

SEQ ID NO: 109 - DNA, 357 bp, Artificial Sequence; identical to SEQ ID NO: 103
  VH

SEQ ID NO: 110 - DNA, 336 bp, Artificial Sequence; identical to SEQ ID NO: 104
  VK

SEQ ID NO: 111 - DNA, 366 bp, Artificial Sequence
  VH
gaggtgcaattggtggagagcggaggagggctcgtgcagcctggaagatctcttaggctgagttgcgctgcatctgggttcacattcaacgactacgccatgcactgggtgaggcaggctcccggcaaagggctggaatgggtgtcaactatctcctggaactccggcagcatcggctacgccgatagcgtcaagggccggtttacaatttcccgcgataacgccaagaagtccctgtacctgcagatgaacagcctgcgggccgaggatactgccctctactactgtgccaaggacattcagtacgggaattactattacgggatggacgtctggggccaggggaccaccgtgacagtcagctcc

SEQ ID NO: 112 - DNA, 321 bp, Artificial Sequence
  VK
gaaatcgtgctgacccagtccccagcaaccctctccctttctcctggagagagagctaccctcagctgtagggcctcacagtctgtctccagttacctggcttggtaccagcagaaacccgggcaggcccctaggttgctgatctacgacgccagcaatagggccactggcatcccagcccggttttccggaagcggcagcgggacagatttcacactcactattagcagcctggagcccgaggacttcgccgtgtactattgccagcagcggtccaactggcccattacatttggccaagggacacgcctggagattaag

SEQ ID NO: 113 - DNA, 363 bp, Artificial Sequence
  VH
caggtccagctgcagcagcccggagccgaactggtcaaacccggggctagcgtgaaaatgtcttgcaaagcaagtggttacacattcacttcctataacatgcactgggtgaagcagacacctgggcgaggtctggaatggatcggcgccatctacccaggcaacggagacactagctataatcagaagtttaaaggaaaggccaccctgacagctgataagtccagctctaccgcttacatgcagctgagttcactgacaagtgaggactcagcagtgtactattgcgcccgttctacctactatggcggagattggtatttcaatgtgtggggcgccggtaccacagtcaccgtgtccgcc

SEQ ID NO: 114 - DNA, 318 bp, Artificial Sequence
  VK
cagattgtcctgagccagagccctgccatcctgtctgctagtcccggcgagaaggtgaccatgacatgcagggcatccagctctgtctcctacatccactggttccagcagaagcccgggagttcacctaaaccatggatctacgctacatccaacctggcaagcggtgtgcctgtcaggttttcaggttccggcagcggaacatcttacagtctgactatttctcgggtggaggccgaagacgccgctacctactattgccagcagtggacctccaatccccctacattcggcggagggactaagctggagatcaaa

SEQ ID NO: 115 - DNA, 363 bp, Artificial Sequence; identical to SEQ ID NO: 113
  VH

SEQ ID NO: 116 - DNA, 318 bp, Artificial Sequence; identical to SEQ ID NO: 114
  VK

SEQ ID NO: 117 - DNA, 366 bp, Artificial Sequence; identical to SEQ ID NO: 111
  VH

SEQ ID NO: 118 - DNA, 321 bp, Artificial Sequence; identical to SEQ ID NO: 112
  VK

SEQ ID NO: 119 - DNA, 363 bp, Artificial Sequence; identical to SEQ ID NO: 113
  VH

SEQ ID NO: 120 - DNA, 318 bp, Artificial Sequence; identical to SEQ ID NO: 114
  VK

SEQ ID NO: 121 - protein, 126 aa, Artificial Sequence
  engineered TCRBeta
LEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEA

SEQ ID NO: 122 - protein, 95 aa, Artificial Sequence
  engineered TCRAlpha
PDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS